(19) United States
(12) Patent Application Publication
(10) Pub. No.: US 2019/0297861 A1
(43) Pub. Date: Oct. 3, 2019

(54) ANIMALS, CELLS, LIGANDS, POLYPEPTIDES & METHODS

(71) Applicant: Kymab Limited, Cambridge (GB)

(72) Inventors: Robert WILLIAMS, Cambridge (GB); E-Chiang LEE, Cambridge (GB); Allan BRADLEY, Cambridge (GB)

(21) Appl. No.: 16/316,994

(22) PCT Filed: Jul. 12, 2017
(86) PCT No.: PCT/GB2017/052046

§ 371 Date: Jan. 10, 2019

(30) Foreign Application Priority Data
Jul. 12, 2016 (GB) 1612117.0
Dec. 20, 2016 (GB) 1621742.4

(51) Int. Cl. A01K 67/027; C07K 14/725; C07K 14/74; C12N 5/0781; A61P 35/00; A61P 37/06; C12N 5/0783; A61K 35/17; G01N 33/68

(52) U.S. Cl. CPC A01K 67/0278; C07K 14/7051; C07K 14/70539; C12N 5/0635; A61P 35/00; A61P 37/06; A61K 38/00; A61K 35/17; G01N 33/68; C07K 2319/30; C07K 2319/00; C07K 2317/31; A01K 2227/105; C12N 5/0636

(57) ABSTRACT

The invention relates inter alia to antigen binding ligands, wherein each ligand comprises a polypeptide comprising a T-cell receptor (TCR) variable domain and an antibody constant domain, non-human vertebrate comprising in its germline a locus for producing a plurality of such antigen binding ligands and cells (eg, B-cells) expressing such ligands.

### CROSS REFERENCE TO RELATED APPLICATIONS

This application is a national stage application under 35 U.S.C. § 371 of International Application No. PCT/GB2017/052046, filed internationally on Jul. 12, 2017, which claims priority to Great Britain Application No. 1612117.00, filed Jul. 12, 2016 and 1621742.4 filed Dec. 20, 2016, the disclosures of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165062000100SEQLIST.TXT, date recorded: Jan. 3, 2019, size: 12 KB).

### FIELD OF THE INVENTION

The invention relates inter alia to antigen binding ligands, wherein each ligand comprises a polypeptide comprising a T-cell receptor (TCR) variable domain and an antibody constant domain, a non-human vertebrate comprising in its germline a locus for producing a plurality of such antigen binding ligands and cells (eg, B-cells) expressing such ligands.

### BACKGROUND

Genetically modified animals have proven to be a powerful tool for the generation of novel therapeutic antibodies. The antibody germline loci comprise variable region gene segments and constant region exons, with the rearranged variable region gene segments encoding the antigen-binding region of an antibody molecule. In a living animal challenged with antigen, B lymphocytes naturally undergo a selection process resulting in cells producing high-affinity, antigen-specific antibodies. These in vivo processes can be exploited to generate antibodies suitable for therapeutic use in humans, from transgenic animals carrying human immunoglobulin variable region gene segments.

The mammalian TCR (T-cell Receptor) and antibody loci share many common structural properties and are thought to have arisen from large-scale duplication events during evolution. For example, both the TCR and antibodies are comprised of variable region and constant region gene segments. Despite possessing basic similarities at the level of germline locus organisation and protein structure, a number of crucial differences exist between TCR and antibody molecules. These differences relate to their functional requirements. One key difference relates to antigen binding affinity and selection. Natural TCRs generally bind to their cognate ligands (eg, pMHC) with weak affinity and fast kinetics, and T-cells undergo both positive and negative selection processes during development to acquire TCR molecules with such properties. In contrast, antibody molecules with desirable properties to an organism, i.e. the provision of effective and specific humoral immunity against pathogens, are generally those which possess very high affinity and specificity for a specific antigenic epitope. B-cells therefore have distinct maturation and selection processes to achieve this. In particular, the process of affinity maturation, during which somatic hypermutation (SHM) may take place at the immunoglobulin loci, is essentially unique to B-cells.

It would be desirable to provide new, engineered means for producing TCR variable domains and repertoires of these.

### BRIEF DESCRIPTION OF THE FIGURES

FIG. 1: Schematic of sRMCE techniques (as described in Lee et al, Nat Biotechnol. 2014, infra) useful to modify the mouse IgH locus via introduction of a region of BAC DNA containing sequence from the human T-cell Receptor Beta (TRB) locus. Mouse ES cells containing a ‘landing pad’ targeted to the endogenous IgH locus, as described by Lee et al, were used. The landing pad incorporates one wild-type LoxP site (black triangle) and one mutant lox5171 site (white triangle). The human TRB locus BAC is modified via the introduction of two sequences as depicted, upstream of the TRBV19 exon and downstream of the TRBJ1-6 exon. The introduced sequences incorporate one wild-type LoxP site and one mutant lox5171 site plus one mutant lox2272 site (grey triangle). The flanking BAC region is inserted directionally in the landing pad by Cre-mediated recombination. Correct integrations enable puromycin expression and positive selection for puromycin-resistant clones as described by Lee et al. Following this step, puromycin-resistant clones possess a piggyBac transposon with both 5′ (PBS′) and 3′ (PB3′) inverted terminal repeats. Expression of piggyBac transposase is applied to clones to excise this transposon; clones which have undergone excision are negatively selected for in medium containing FIAU.

Eμ: mouse intronic Eμ enhancer; Sμ: heavy-chain switch sequence.

FIG. 2: Schematic of initial “Locus#1” chimeric IgH locus which incorporates a region of human TRB locus genomic sequence, following sRMCE steps described for FIG. 1. Annotations are the same as for FIG. 1.

FIG. 3: Schematic of a mouse IgH locus which will be modified to include a complete repertoire of functional human TRB variable region segments. Functional TRBV exons between TRBV5-1 and TRBV18 are not shown. Annotations are the same as for FIG. 1.

FIG. 4: Schematic of sRMCE techniques (adapted from Lee et al, Nat Biotechnol. 2014) used, in one embodiment of the invention, to modify the mouse Igκ locus via introduction of a region of BAC DNA containing sequence from the human T-cell Receptor Alpha (TRA) locus. sRMCE methods are identical to those described in FIG. 1, with the exception that the mouse ES cells used carry a ‘landing pad’ targeted to the endogenous Igκ locus, as described by Lee et al. The human BAC insert has a deleted region (marked on figure) between the TRAV41 exon and the TEA element.

TEA: T Early α element; iEκ: mouse kappa intronic enhancer; 3′Eκ: mouse kappa 3′ enhancer.

FIG. 5: Schematic of a mouse Igκ locus which will be modified to include a complete repertoire of functional human TRA variable region segments. Functional TRBV exons between TRAV3 and TRAV23 are not shown. Annotations are the same as for FIG. 4.

FIG. 6: Schematic of a mouse Igκ locus which has been modified to incorporate a recombined human V-J variable domain coding unit, replacing the endogenous Igκ J region exons. The DNA targeting construct incorporates a transposon-flanked selection cassette and a recombined human TRAV17-J33 coding DNA sequence, plus 1300 bp of human genomic sequence upstream of the TRAV17 gene segment and 200 bp of sequence downstream of TRA33 gene segment.

The Igκ locus is annotated as in FIG. 4, other annotations plus Puromycin and PiggyBac selection methods are as described in FIG. 1. HA: homology arm.

FIGS. 7A-7C: Human TCR loci (from world wide web.IMGT.org).

FIG. 8: Variable (V) gene segment usage among unique transcript sequences obtained from cellular RNA obtained from the “CD19+ B cells FACS sorted bone marrow” cell sample. Bars show numbers of unique sequences which aligned to each human TCRβ Variable gene segment.

FIG. 9: Joining (J) gene segment usage among unique transcript sequences obtained from cellular RNA obtained from the “CD19+ B cells FACS sorted bone marrow” cell sample. Bars show numbers of unique sequences which aligned to each human TCRβ Joining gene segment.

FIG. 10: CDR3 sequence length (in amino acids) among unique transcript sequences obtained from cellular RNA obtained from the “CD19+ B cells FACS sorted bone marrow” cell sample. Bars show numbers of unique sequences possessing CDR3 regions (defined as described in the methods section) of given lengths. CDR3 lengths followed a normal distribution curve which resembled that of a human TCRβ CDR3 profile (Blood. 2009 Nov. 5; 114(19): 4099-4107).

FIGS. 11-14: Gene segment nucleotide deletions occurring in unique transcript sequences obtained from cellular RNA obtained from the “CD19+ B cells FACS sorted bone marrow” cell sample. Nucleotide deletions were calculated by aligning each sequence to its closest matching V, D and J gene segment and adding up the number of missing/non-aligning nucleotides at each gene segment sequence end, compared with the original germline sequence.

FIGS. 15 & 16: Gene segment nucleotide insertions occurring in unique transcript sequences obtained from cellular RNA obtained from the “CD19+ B cells FACS sorted bone marrow” cell sample. Nucleotide insertions were calculated by aligning each sequence to its closest matching V, D and J gene segment and adding up the number of non-aligning (i.e. additional non-germline encoded) nucleotides at each junction.

### STATEMENT OF INVENTION

In a first configuration, the invention provides:

A non-human vertebrate comprising in its germline a locus for producing a plurality of antigen binding ligands, wherein each ligand comprises a polypeptide comprising a T-cell receptor (TCR) variable domain and an antibody constant domain, the ligand comprising an antigen binding site wherein the binding site comprises the variable domain, wherein the locus comprises
a. a T-cell receptor (TCR) variable region comprising (in 5′ to 3′ direction) one or more TCR V gene segments; optionally one or more D gene segments; and one or more TCR J gene segments, wherein the variable region is capable of rearranging to produce a rearranged VDJ or VJ; and
b. an antibody constant region comprising one or more antibody C gene segments;
   wherein the variable region is operably linked upstream of the antibody constant region whereby B-cells of the vertebrate express said polypeptides each comprising a TCR variable domain and an antibody constant domain.

In a second configuration, the invention provides:

A method of producing one or more polypeptides, wherein each polypeptide comprises a T-cell receptor (TCR) variable domain and an antibody constant domain, the method comprising
a. providing a vertebrate according to the invention;
b. immunising the vertebrate with an antigen (optionally to effect CSR and SHM in the vertebrate), whereby a plurality of polypeptides comprising TCR V domains and antibody constant domains are expressed by B-cells of the vertebrate; and
c. selecting one or more B-cells capable of expressing a said polypeptide; selecting one or more of said polypeptides; isolating one or more TCR V domains thereof; or isolating one or more nucleotide sequences each encoding a TCR V domain of a said expressed polypeptide;
d. wherein the polypeptide(s) or TCR V domain(s) of (c) specifically binds to the antigen; and
e. optionally expressing said polypeptide(s) or TCR V domain(s).

In an example, this is useful for producing affinity matured TCR V domains and nucleotide sequences thereof. The nucleotide sequence can be inserted, for example, in an expression vector or a T-cell genome for expression of the TCR V domain encoded thereby.

In a third configuration, the invention provides:

A method of using a non-human vertebrate to select for an affinity matured TCR variable domain or a nucleotide sequence encoding an affinity matured TCR variable domain, wherein the variable domain is capable of expression in vivo in a vertebrate, the method comprising
a. providing a non-human vertebrate wherein at least one antibody heavy chain locus of the vertebrate comprises a first TCR variable region upstream of the antibody heavy chain constant region for expressing first mRNA transcripts encoding polypeptides comprising an affinity matured TCR V domain and an antibody C domain; at least one antibody light chain (eg, kappa) locus of the vertebrate comprises a second TCR variable region upstream of the antibody light chain locus for expressing second mRNA transcripts encoding polypeptides comprising a TCR V domain and an antibody C domain;
b. immunising the vertebrate with an antigen to effect CSR and SHM in the vertebrate, whereby a plurality of antigen-specific TCR-Ig ligands comprising affinity matured TCR V domains are expressed by B-cells of the vertebrate; and
c. selecting one or more B-cells capable of expressing a said TCR-Ig; selecting one or more of said TCR-Ig; isolating one or more affinity matured TCR V domains thereof; or isolating one or more nucleic acid sequences (eg, mRNA or DNA) each encoding an affinity matured TCR V domain of an expressed TCR-Ig;
d. wherein the TCR-Ig(s) or TCR V domain(s) of (c) specifically bind to the antigen; and e. Optionally expressing the one or more TCR-Igs or one or more TCR V domains.

In a fourth configuration, the invention provides:

A plurality of B-cells or hybridoma cells that express a plurality of different affinity matured TCR variable domains, wherein one or more of the variable domains specifically binds to an antigen.

In a fifth configuration, the invention provides:

A plurality of mammalian cells that express a plurality of at least 10 different affinity matured TCR variable domains, wherein one or more of the TCR variable domains specifically binds to an antigen.

In a sixth configuration, the invention provides:

A TCRV-Ig comprising a TCR variable domain obtained or obtainable by the method of the invention, wherein the TCRV-Ig specifically binds to an antigen (eg, pMHC antigen).

In a seventh configuration, the invention provides:

A TCR variable domain obtained or obtainable by the method of the invention, wherein the TCR V domain specifically binds to antigen (eg, pMHC antigen).

In an embodiment, the TCR V domain is comprised by an antigen-specific ligand (eg, a TCRV-Ig according to the invention) for use in a method of treating or preventing a disease in a human or non-human animal patient, the method comprising administering the ligand to the patient wherein the ligand specifically binds the antigen for antagonising the antigen or killing cells expressing the antigen whereby the disease or condition is treated. In an example, the method is a method of adoptive T-cell transfer (ACT), comprising administering engineered T-cells to the patient, wherein the T-cells surface-express the TCR V domain.

In another embodiment, the TCR V domain is fused to an antibody gamma (eg, gamma-1) constant region, wherein the gamma constant region provides ADCC or CDC effector function for cell killing in the patient, wherein the patient comprises cells expressing the antigen and the cells are killed thereby treating the disease or condition, wherein the disease or condition is a cancer; autoimmune disease or condition; inflammatory disease or condition; or viral infection. In an example, the method is a method of adoptive T-cell transfer (ACT), comprising administering engineered T-cells to the patient, wherein the T-cells surface-express the TCR V domain.

In an eighth configuration, the invention provides:

A multispecific ligand comprising
a. a first binding site comprising a TCR Vα domain/TCR Vβ domain pair or a TCR VT domain/TCR Vδ domain pair, wherein one or both of the V domains is a TCR V domain according to the invention, wherein the first binding site specifically binds an antigen (eg, pMHC antigen or a tumour associated antigen (TAA)); and
b. a second binding site that specifically binds to a T-cell or NK cell surface antigen.

In a ninth configuration, the invention provides:

A nucleic acid comprising a nucleotide sequence encoding the TCRV-Ig, TCR V domain or ligand of the invention, optionally comprised by an expression vector for expressing the TCRV-Ig, TCR V domain or ligand.

In a tenth configuration, the invention provides:

An engineered immune cell comprising a TCRV-Ig, TCR V domain or ligand of the invention, or the nucleic acid, wherein the immune cell expresses the TCRV-Ig, TCR V domain or ligand, eg, on the cell surface.

In an eleventh configuration, the invention provides:

A CAR-T or CAR-NK cell comprising a chimaeric antigen receptor (CAR), the receptor comprising an extracellular moiety, a transmembrane moiety and an intracellular signalling moiety, wherein the extracellular moiety comprises the TCRV-Ig or TCR V domain of the invention or comprises a first binding site of the ligand of the invention and/or the cell genome comprises the nucleotide sequence of the invention for expressing the TCRV-Ig, TCR V domain or ligand first binding site as part of the extracellular moiety of the receptor.

In a twelfth configuration, the invention provides:

A method of identifying an antigen, the method comprising
a. carrying out the method of the third configuration to select a nucleotide sequence encoding an affinity matured TCR V domain, wherein the antigen is a cell surface-expressed antigen (eg, pMHC expressed on a cancer cell or virally-infected cell);
b. using the selected sequence to produce a second nucleotide sequence encoding a ligand comprising an antigen binding site, wherein the binding site comprises a said affinity matured TCR V domain and binds to the antigen with a binding affinity (KD) of less than 100 nM as determined by surface plasmon resonance (SPR);
c. using the second sequence to express copies of the ligand;
d. providing a plurality of cells comprising cell surface-expressed epitopes (eg, a plurality of cancer cell or virally-infected cells expressing pMHCs);

e. exposing the plurality of cells to copies of said ligand;

f. selecting one or more cells that are specifically bound by ligand; and

g. identifying the cell surface antigen of a said cell that is specifically bound by a ligand; and

h. optionally expressing the antigen or producing nucleic acid comprising nucleotide sequence encoding the antigen (eg, encoding the peptide of a pMHC antigen).

In a thirteenth configuration, the invention provides:

A method of transcribing a rearranged TCR variable region sequence in a non-human vertebrate or cell, the method comprising ectopically transcribing said rearranged TCR variable region sequence in said vertebrate or cell (eg, a B-cell).

In a fourteenth configuration, the invention provides:

A method of rearranging TCR V, D and J (or V and J) gene segments to produce a rearranged TCR variable region sequence in a non-human vertebrate or cell, the method comprising ectopically rearranging said gene segments in said vertebrate or cell (eg, a B-cell), whereby a transcribable rearranged TCR variable region sequence is produced.

In an example of the 13^{th} and 14^{th} configurations, the vertebrate or cell is any vertebrate or cell of the invention herein. For example, the vertebrate or cell comprises a genome that comprises a T-cell receptor (TCR) variable region comprising (in 5′ to 3′ direction) one or more TCR V gene segments; optionally one or more D gene segments; and one or more TCR J gene segments, wherein the variable region is capable of rearranging to produce a rearranged VDJ or Vi, or wherein the variable region has been rearranged (in the 13^{th} configuration), wherein the TCR V region is at an ectopic genomic position (ie, a non-natural position in the genome). For example, the V region is outside a TCR locus position, eg, the V region is comprised by an Ig locus (eg, an IgH, Igκ or Igλ locus).

In a fifteenth configuration, the invention provides:

A non-human vertebrate or non-human vertebrate cell that comprises a rearranged TCR V region that is expressible to produce one or more in-frame transcripts comprising a TCR V region nucleotide sequence spliced to a nucleotide sequence encoding an Ig constant region (eg, a Cμ region).

In a sixteenth configuration, the invention provides:

A plurality of B-cells (eg, mouse B-cells) comprising immunoglobulin loci that comprise recombined TCR variable regions, wherein the variable regions comprise TCR gene segment junctional mutation.

In a seventeenth configuration, the invention provides:

A non-human vertebrate that comprises a plurality of B-cells, the B-cells comprising immunoglobulin loci that comprise recombined TCR variable regions, wherein the variable regions comprise TCR gene segment junctional mutation.

In a nineteenth configuration, the invention provides:

A non-human vertebrate or a non-human vertebrate cell (eg, a mouse or a mouse cell) that comprises a rearranged TCRB variable region that is ectopically positioned in the genome of the vertebrate of cell, wherein the vertebrate or cell expresses TCRB V domains comprising most commonly a CDR3 length of 11, 12 or 13 amino acids, eg, of 12 amino acids.

### DETAILED DESCRIPTION

The invention relates inter alia to antigen binding ligands, wherein each ligand comprises a polypeptide comprising a T-cell receptor (TCR) variable domain and an antibody constant domain, non-human vertebrate comprising in its germline a locus for producing a plurality of such antigen binding ligands and cells (eg, B-cells) expressing such ligands. The invention also relates to affinity matured TCR V domains and means for producing these and using these, eg, in medicine or diagnostics. The invention also relates to TCR V domains and means for producing these and using these, eg, in medicine or diagnostics, wherein each TCR V domain comprises a recombined variable region (a VDJ or VJ region) and one or more VDJ or VJ junctional mutations obtainable by recombination of the VDJ or VJ at an IgH or IgL locus in a non-human vertebrate, eg, a rodent, rat or mouse.

The invention also relates to a method of ectopically transcribing a rearranged TCR variable region sequence in a non-human vertebrate, eg, a mammal, rodent, rat or mouse, or in a non-human vertebrate cell (eg, in a B-cell). In an embodiment, a plurality of said reararanged variable regions are transcribed, wherein said plurality comprises different said rearranged variable regions. In an example, the invention provides a plurality of said rearranged TCR variable region sequences, wherein said plurality comprises different said rearranged variable regions. In an embodiment, each variable region is comprised by an expression vector (eg, in a host cell, eg, a CHO or HEK293) for expression of TCR V domains. By “ectopically” it is meant that the rearranged TCR variable region is transcribed from a non-natural genomic location, ie, from a genomic position outside the respective endogenous TCR locus (eg, TCRβ locus) of the genome of a said non-human vertebrate, eg, from a position outside all of the TCR loci of said genome. Optionally, TCR VDJ or VJ gene segments are rearranged ectopically to produce said rearranged TCR variable region in said vertebrate. In an embodiment, the TCR variable region is a TCRα variable region that is transcribed from a genomic position that is not at a TCRα locus. In an embodiment, the TCR variable region is a TCRβ variable region that is transcribed from a genomic position that is not at a TCRβ locus. In an embodiment, the TCR variable region is a TCRγ variable region that is transcribed from a genomic position that is not at a TCRγ locus. In an embodiment, the TCR variable region is a TCRδ variable region that is transcribed from a genomic position that is not at a TCRδ locus. In any of these embodiments, optionally the position is not in any TCR locus and/or the position is at an Ig locus (eg, an IgH, Igκ or Igλ locus). Thus, these embodiments employ the “ectopic” feature of the invention as per this and the next paragraph.

The invention further provides a method of rearranging TCR V, D and J (or V and J) gene segments to produce a rearranged TCR variable region sequence in a non-human vertebrate or cell, the method comprising ectopically rearranging said gene segments in said vertebrate or cell (eg, a B-cell), whereby a transcribable rearranged TCR variable region sequence is produced. In an example the method further comprises transcribing said rearranged TCR variable region sequence to produce mRNA transcripts encoding a TCR V domain and optionally an antibody constant domain (eg, a Cμ). Optionally, the method further comprises isolating the TCR V domain-encoding sequence of said one or more of said transcripts, and optionally inserting the sequence into an expression vector (eg, a CHO or HEK293 expression vector) for expression of TCR V domains. The invention further provides a cell line (eg, a CHO or HEK293 cell line) comprising said sequence or said vector. The method further provides a method of producing a TCR V domain, the method comprising expressing said V domain from said cell line, sequence or vector. Herein “recombine” and “reararrange” in respect of gene segments are used interchangeably.

The invention, in one aspect, harnesses expression of TCR variable domains in the context of elements of antibody loci (such as IgH and/or IgL loci). This provides for the non-natural configuration of B-cells that express TCR variable domains, rather than the wild-type configuration where TCR variable domains are expressed by T- but not B-cells. This provides the useful possibility of using established techniques to screen, sort and select B-cells, as well as established methods of making and using B-cell hybridomas or immortalised B-cells in screening and production of binding ligands comprising TCR variable domains.

In an embodiment, this enables harnessing of one or more mechanisms characteristic of B-cell antibody loci rearrangement and maturation. Thus, in examples the invention harnesses gene segment junctional mutation (producing junctional diversity in recombined TCR V regions) class-switch recombination (CSR) and/or somatic hypermutation (SHM) at one or more antibody loci in B-cells, where those loci comprise TCR variable region gene segments. In this way, junctional mutation, CSR and/or SHM can be employed in B-cells to mature TCR variable domains. Thus, this provides the possibility to provide TCR variable domains and repertoires thereof with epitope-binding affinities and diversities produced by harnessing B-cell processes.

By using in vivo expression of TCR variable domains in non-human vertebrates (eg, in a mammal, rodent, mouse or rat), the invention in an embodiment exploits the in vivo system as a way of selecting for affinity matured TCR variable domains that can be properly folded and expressed in a vertebrate (eg, mammalian) system. This is useful for subsequent development and use of the domains in antigen binding ligands, for example, for human medicine.

The invention, in an embodiment, also provides means for in vivo production and selection of TCR-Ig ligands, for example, for producing one or more TCR-Ig comprising an affinity matured TCR variable domain. As used herein, such “TCR-Ig” comprise one or more polypeptides, each comprising (in N- to C-terminal direction) a TCR variable domain and an antibody constant domain (eg, Fc or CL). TCR-Ig are useful, for example, as medicaments, diagnostic tools or for providing TCR variable domains that can be used for producing medicaments or diagnostic tools. By using in vivo expression of TCR-Ig in non-human vertebrates, the invention in an embodiment exploits the in vivo system as a way of selecting for TCR variable domains that can be properly folded and expressed in a vertebrate (eg, mammalian) system when part of a polypeptide also comprising one or more antibody domains. This combination is non-naturally occurring and such in vivo selection enables one to obtain a plurality of TCR-Ig that can nonetheless be expressed.

Provided herein are novel designs for the generation in B-cells of TCR-Ig and TCR variable domains. This is useful, eg, for producing TCR variable domains that are affinity matured providing the possibility of TCR V domain antigen binding affinities that are stronger than typical for naturally-occurring TCR V domains (the binding affinity of natural TCR-pMHC interactions is around KD˜0.1-500 μM). Potential applications of such TCR V domains are for producing engineered human immune cells, such as T- or NK-cells bearing cell-surface antigen receptors comprising such domains. For example, the TCR V domains of the invention are useful for producing engineered TCRs or CARs (Chimaeric Antigen Receptors) that are expressed on engineered human T- or NK-cells for human medical use. TCR-Ig and TCR V domains of the invention may, for example, possess novel antigen recognition sites. In particular, such molecules may possess an intrinsic ability to prefer binding peptide antigens presented in the context of peptide-MHC (pMHC).

B-cell production of TCR-Ig and TCR variable domains is also or alternatively useful as it enables the employment of routine, well-established methods for harvesting, screening, selecting and propagating B-cells and hybridomas, eg, for the production of medicines or diagnostic tools.

In an embodiment, the invention involves the replacement of variable region gene segments at one or more endogenous immunoglobulin loci in a non-human vertebrate with TCR variable region gene segments, such as unrearranged segments of a human germline TCR locus or a rearranged TCR V(D)J segments. In another example, the replacement is a functional replacement achieved by insertion of the TCR variable region gene segments into the vertebrate genome at a location outside the endogenous Ig loci. This may be effected using standard random transgene insertion (eg, by pronuclear injection of one or more transgenes into a zygote). One or more endogenous Ig loci (eg, IgH and/or Igλ loci) may be inactivated for endogenous antibody variable domain expression, such as by deletion of the J gene segments in the loci and/or insertion of a neo or other selection marker sequence in the variable region, as is routine in the art. In another example, antibody gene segments may be supplemented with (rather than replaced by) TCR gene segments at one or more endogenous Ig loci in the vertebrate.

In a configuration, the invention provides:

A non-human vertebrate comprising in its germline a locus for producing a plurality of antigen binding ligands, wherein each ligand comprises a polypeptide comprising a T-cell receptor (TCR) variable domain and an antibody constant domain, the ligand comprising an antigen binding site wherein the binding site comprises the variable domain, wherein the locus comprises
a. a T-cell receptor (TCR) variable region comprising (in 5′ to 3′ direction) one or more TCR V gene segments; optionally one or more D gene segments; and one or more J gene segments, wherein the variable region is capable of rearranging to produce a rearranged VDJ or VJ; and
b. an antibody constant region comprising one or more antibody C gene segments;
   wherein the variable region is operably linked upstream of (ie, 5′ of) the antibody constant region whereby B-cells of the vertebrate express said polypeptides each comprising a TCR variable domain and an antibody constant domain.

In an example, the TCR variable region is at an endogenous antibody locus. In an embodiment, the constant region comprises an endogenous antibody constant gene segment. The constant region may comprise one or more constant gene segments of a species (eg, human or rodent (such as mouse or rat)) that is different to the species of the vertebrate. For example, all of the constant gene segments of the constant region are human gene segments. For example, the antibody locus is an IgH locus and the constant region comprises an endogenous Cmu constant gene segment and human Cgamma constant gene segments. For example, the antibody locus is an IgH locus and comprises an endogenous Smu operably linked upstream of a Cmu (eg, an endogenous Cmu). For example, the locus comprises an endogenous intronic enhancer (eg, Emu when the antibody locus is an IgH; or iEκ when the antibody locus is an 100. For example, the locus comprises an endogenous 3′ enhancer of the antibody locus.

In alternative, the TCR variable region is not at an endogenous antibody locus, eg, it has been randomly inserted into the germline genome of the vertebrate. In an embodiment, therefore, the constant region comprises an exogenous antibody constant gene segment (and does not comprise an endogenous antibody constant gene segment). For example, the exogenous constant gene segment may be of the same or a different species to the vertebrate, eg, it is a human or rodent (such as mouse or rat) constant gene segment. In an embodiment, the TCR variable region is at the Rosa26 locus.

In an example, the vertebrate comprises first and second loci of the invention, wherein (i) the first locus comprises a said TCR variable region at an endogenous IgH locus; and (ii) the second locus is provided by a transgene comprising an exogenous antibody constant region, eg, wherein the transgene has been randomly inserted into the germline genome of the vertebrate (eg, using standard pronuclear injection into a zygote or another convention method for random insertion of antibody loci transgenes). In an example, therefore, the first locus is produced by insertion of TCR variable region gene segments at an endogenous IgH locus of an embryonic stem cell (ES cell) or iPS cell of the species of said non-human vertebrate (eg, a mouse or rat cell). Subsequently, the second locus is introduced as a transgene into the genome of the cell (or a progeny cell or zygote thereof), wherein the transgene is inserted into the genome of the cell or zygote. Using conventional methods, a non-human vertebrate is then developed from the cell, zygote or a progeny thereof. Alternatively, as is known in the art, separate non-human vertebrates (eg, mice) can be bred to together to combine the loci in the germline genome of a progeny, by using a first vertebrate whose germline comprises the first locus and a second vertebrate whose genome comprises the second locus, wherein a progeny thereof comprises a germline genome comprising both loci for expression of TCR V domains from the loci. In an alternative to the use of a transgene for the second locus, the second locus can instead comprise the respective TCR V region at an endgoenous IgL locus (eg, a kappa or lambda locus).

In an embodiment, the vertebrate is homozygous for the or each locus comprising TCR variable gene segments. For example, the vertebrate is homozygous for an endogenous IgH locus comprising TCR V gene segments. In an alternative, the vertebrate is heterozygous for the or each such locus.

In an example, one or more of the loci comprising TCR V gene segments comprises DNA from more than one species (eg, human and rodent (such as mouse or rat) DNA).

The skilled person is familiar with identifying and isolating B-cells from non-human vertebrates (eg, mice and rats), for example by identifying B-cell surface markers and using routine FACS sorting.

In an example, the or each ligand is a TCR-Ig. Embodiments of TCR-Ig are as follows:—

An antigen binding ligand comprising
(i) A polypeptide that comprises a TCRα V domain and one or more antibody constant domains (eg, a CH1, CH2, CH3, Fc, Cκ or Cλ), optionally wherein the V domain is human and the C domain is human or rodent (such as mouse or rat);
(ii) A polypeptide that comprises a TCRβ V domain and one or more antibody constant domains (eg, a CH1, CH2, CH3, Fc, Cκ or Cλ), optionally wherein the V domain is human and the C domain is human or rodent (such as mouse or rat);
(iii) A polypeptide that comprises a TCRγ V domain and one or more antibody constant domains (eg, a CH1, CH2, CH3, Fc, Cκ or Cλ), optionally wherein the V domain is human and the C domain is human or rodent (such as mouse or rat);
(iv) A polypeptide that comprises a TCRδ V domain and one or more antibody constant domains (eg, a CH1, CH2, CH3, Fc, Cκ or Cλ), optionally wherein the V domain is human and the C domain is human or rodent (such as mouse or rat);
(v) A first polypeptide that comprises a TCRα V domain and an antibody Fc (eg, a human or rodent (such as mouse or rat) Fc); associated with a second polypeptide that comprises a TCRβ V domain and an antibody Cλ (eg, a human or rodent (such as mouse or rat) Cλ); optionally wherein each V domain is human;
(vi) A first polypeptide that comprises a TCRα V domain and an antibody Fc (eg, a human or rodent (such as mouse or rat) Fc); associated with a second polypeptide that comprises a TCRβ V domain and an antibody Cκ (eg, a human or rodent (such as mouse or rat) Cκ); optionally wherein each V domain is human;
(vii) A first polypeptide that comprises a TCRβ V domain and an antibody Fc (eg, a human or rodent (such as mouse or rat) Fc); associated with a second polypeptide that comprises a TCRα V domain and an antibody Cλ (eg, a human or rodent (such as mouse or rat) Cλ); wherein the V domains form an antigen binding site; optionally wherein each V domain is human;
(viii) A first polypeptide that comprises a TCRβ V domain and an antibody Fc (eg, a human or rodent (such as mouse or rat) Fc); associated with a second polypeptide that comprises a TCRα V domain and an antibody Cλ (eg, a human or rodent (such as mouse or rat) Cλ); wherein the V domains form an antigen binding site; optionally wherein each V domain is human;
(ix) A first polypeptide that comprises a TCRγ V domain and an antibody Fc (eg, a human or rodent (such as mouse or rat) Fc); associated with a second polypeptide that comprises a TCRδ V domain and an antibody Cκ (eg, a human or rodent (such as mouse or rat) Cκ); wherein the V domains form an antigen binding site; optionally wherein each V domain is human;
(x) A first polypeptide that comprises a TCRγ V domain and an antibody Fc (eg, a human or rodent (such as mouse or rat) Fc); associated with a second polypeptide that comprises a TCRδ V domain and an antibody Cλ (eg, a human or rodent (such as mouse or rat) Cλ);

wherein the V domains form an antigen binding site; optionally wherein each V domain is human;
(xi) A dimer of any one of (i) to (x).

In an example, the or each TCR-Ig is a “TCRV-Ig”, ie, a TCR-Ig comprising one or more polypeptides, each polypeptide comprising (in N- to C-terminal direction) a TCR V domain and an antibody C domain without a TCR C domain between the TCR V domain and antibody C domain. Thus, in each of the polypeptides recited in options (i) to (xi) above, there is for example no TCR C domain between the TCR V domain and the C or Fc.

In an option, each polypeptide of (i) to (xi) or TCRV-Ig may comprise one or more further Ig or non-Ig domains (but in the case of TCRV-Ig, not a TCR C domain) between the TCR V domain and the C or Fc. In an example, the further domain(s) comprise an antigen binding site, eg, that has specificity for an antigen or epitope that is different to that specifically bound by the TCR V domain in the ligand or polypeptide. Thus, this provides for multi-, bi- or tri-specific or valent ligands and polypeptides.

One or more of the TCR V domains is comprised by an antigen binding site of the ligand. In an embodiment, one or more of the polypeptides comprises a further antigen binding site, eg, the ligand comprises an antibody V domain or VH/VL binding site, or the ligand comprises an FcAb binding site (eg, comprised by the C region of the ligand). In this way, the ligand may be a multi-, bi- or tri-specific ligand that is capable of binding to two or more (eg, two or three) different antigens or different epitopes on the same antigen species.

Said plurality of ligands expressed by the vertebrate can, for example, comprise TCR V gene segments for producing a theoretical diversity of at least 10, 100, 1000, 10⁴, 10⁵, 10⁶, 10⁷, 10⁸, 10⁹, 10¹⁰ or 10¹¹ different VDJ and/or VJ combinations in the vertebrate with or without immunisation with an antigen.

In an example, the vertebrate is a rodent, eg, a mouse or rat. In other examples, the vertebrate is a bird (eg, chicken), fish (eg, shark or zebrafish), livestock animal (eg, a cow, sheep, pig or goat), rabbit or Camelid (eg, a llama, alpaca or camel). In an embodiment, the mouse strain is 129 (or a 129 hybrid), C57BL6 (or C57BL6 hybrid), derived from an AB2.1, AB2.2, JM8, BALB/c, or F1H4 ES cell line.

In an example, the TCR V region comprises V, D and J gene segments, wherein the V and D gene segments comprise compatible RSS sequences and the D and J segments comprise compatible RSS sequences, wherein the variable region is capable of recombination to produce a rearranged VDJ sequence that encodes a TCR V domain. The D segments may be TCR or antibody D segments (eg, TCRBD or DH, such as when the V is TCRBV). The J segments may be TCR or antibody J segments (eg, TCRBJ or JH, such as when the V is TCRBV).

In an example, the TCR V region comprises V and J gene segments (and no Ds), wherein the V and J segments comprise compatible RSS sequences, wherein the variable region is capable of recombination to produce a rearranged VJ sequence that encodes a TCR V domain. The J segments may be TCR or antibody J segments (eg, TCRAJ or JL, such as when the V is TCRAV).

In an example, the TCR V region comprises at least 2, 3, 4, 5, 6, 7, 8, 9, 10, 11, 12, 13, 14, 15, 16, 17, 18, 19, 20, 30 or 40 different TCR V gene segments (eg, which are all human). Additionally or alternatively, in an example, the TCR V region comprises at least 2, 3, 4, 5, 6, 7, 8, 9, 10, 11, 12, 13, 14, 15, 16, 17, 18, 19, 20, 30 or 40 different TCR J gene segments (eg, which are all human). Additionally or alternatively, in an example, the TCR V region comprises at least 2, 3, 4, 5, 6, 7, 8, 9, 10, 11, 12, 13, 14, 15, 16, 17, 18, 19, 20, 30 or 40 TCR D gene segments (eg, which are all human). In an embodiment, the gene segments (eg, TCRBVs and/or TCRBJs) are of a first species (eg, human) and are in germline order with respect to TCR loci (eg, TCRB loci) in the first species. In an embodiment, the TCR V region comprises inter-gene segment sequences between the gene segments (eg, TCRBVs and/or TCRBJs) that are found in respective TCR loci (eg TCRB loci).

In an example, the TCR V region comprises a fragment of a TCRA (ie, alpha), B (ie, beta), G (ie, gamma) or D (ie, delta) locus from (and including) the 3′-most V to (and including) the 5′-most J. In an example, the TCR V region comprises a fragment of a TCRA, B, G or D locus from (and including) the 3′-most V to (and including) the 5′-most J. In an embodiment, the TCRA, B, G or D locus is a human locus.

Additionally or alternatively, the C region comprises a fragment of an IgH locus from an including the Cmu to (and optionally including) the 5′-most gamma-1 exon; optionally including all gamma-1 CH1-3 or CH1-M2 exons. In an alternative, the fragment is from and including Smu or Emu. In an alternative, the fragment is from a point within the first 400, 500, 600, 700 800 or 900 nucleotides of the IgH J-C intron, wherein the fragment comprises intronic DNA 5′ of and contiguous with Emu and Cmu. In an embodiment, the IgH locus is an endogenous IgH locus of the non-human vertebrate, eg a rodent (eg, mouse or rat) locus. In an alternative, the locus is of the same species as the vertebrate but is an exogenous locus (eg, from a genome that is different from that of the vertebrate). In an alternative, the locus is a human locus. In an alternative, the locus is a synthetic locus.

In an example, the constant region comprises at least one IgH C gene segment, eg, a Cmu gene segment, and optionally also one or more of an alpha, delta, epsilon and gamma (eg, gamma-1) C gene segment. In an embodiment, the constant region comprises a Cmu and a Cgamma (eg, gamma-1, human gamma-1, mouse gamma-1 or rat gamma-1 C segment). One or both of the Cmu and Cgamma can be endogenous to the vertebrate; eg, the Cmu is endogenous and Cgamma is endogenous or human. In an example, the gene segments of the C region are in germline order of C segments found in an IgH or IgL locus of a human, rodent, rat or mouse genome. In an example, the gene segments of the C region are in germline order of C segments found in an IgH or IgL locus of a mouse genome. This order is known to the skilled addressee.

In an example, the antibody C gene segment(s) are endogenous segments of the vertebrate, optionally wherein the constant region is an endogenous heavy chain constant region at an endogenous heavy chain locus, or an endogenous light chain (kappa or lambda) constant region at an endogenous light chain locus. Thus, when the vertebrate is a mouse, the C segment(s) are those on chromosome 12 (for IgH C segment(s)), 6 (for Igκ C segment(s)) or 16 (for Igλ C segment(s)).

In an example, the locus of the invention does not comprise antibody V region gene segments.

For example, the D and J gene segments are TCR gene segments. For example, the V, D and J are all TCRB gene segments. For example, the V, D and J are all TCRD gene segments. For example, the V and J are all TCRA gene segments, wherein the variable region does not comprise said D gene segments. For example, the V and J are all TCRG gene segments, wherein the variable region does not comprise said D gene segments.

In an example, the locus comprises (i) the functional TCRBV, D and J gene segments of a human TCRβ locus from TCRBV19 to TCRBJ1-1 inclusive, and optionally up to TCRBJ1-6; or (ii) the functional TCRAV and J gene segments of a human TCRα locus from TCRAV24 to TCRAJ61 inclusive, and optionally up to TCRAJ1. As the skilled addressee will know, functional gene segments are denoted as green boxes in the locus representations shown in the IMGT Repertoire database (see http://www.imgt.org/IMGTrepertire/LocusGenes/#h.1_6).

In an example, a locus of the invention comprises human TCRBV19, TCRBV20-1, TCRBV24-1, TCRBV25-1, TCRBV27, TCRBV28 and TCRBV29-1. For example, the TCRBV gene segments are at an Ig locus, eg, an IgH locus, eg, an endogenous IgH locus of the vertebrate or cell. In an example, the locus further comprises one or TCRBD gene segments and TCRBJ1-1, TCRBJ1-2, TCRBJ1-3, TCRBJ1-4, TCRBJ1-5 and TCRBJ1-6. In an example, the TCR V gene segment is selected from the group consisting of TRBV 19*01, 20-1*02, 24-1*01, 25-1*01, 27*01, 28*01 and 29-01*01. For example, the TCR V is 20-1 (eg, 20-1*02). For example, the TCR V is 27 (eg, 27*01). In an example, the TCR J gene segment is selected from the group consisting of TRBJ 1-1*01, 1-2*01, 1-3*01, 1-4*01, 1-5*01 and 1-6*01. For example, the TCR J is TCRBJ 1-5 (eg, 1-5*01).

In an example,
a. the one or more V gene segments are TCRAV segments and the one or more J gene segments are TCRAJ gene segments;
b. the one or more V gene segments are TCRBV segments and the one or more J gene segments are TCRBJ gene segments;
c. the one or more V gene segments are TCRCV segments and the one or more J gene segments are TCRCJ gene segments; or
d. the one or more V gene segments are TCRDV segments and the one or more J gene segments are TCRDJ gene segments.

In an example, alternatively to an unrearranged variable region, the locus of the invention comprises a rearranged TCR VJ (eg, VαJα or VγJγ) or VDJ (eg, VβDβJβ or VδDδJδ). The rearranged VJ or VDJ may be human or synthetic, for example. In an example, the vertebrate comprises such a rearranged TCR VJ operably linked upstream of an endogenous CL constant region (eg, at a mouse or rat endogenous kappa locus) and an unrearranged V-D-J region operably linked upstream of an endogenous IgH constant region (eg, at a mouse or rat endogenous IgH locus). In this way, a common light chain can be expressed using the rearranged VJ (eg, optionally wherein the vertebrate cannot mutate the VJ sequence) that can paired with a repertoire of TCR-Ig heavy chains expressed from the other locus. This is useful for producing bispecific TCR V-containing ligands, eg, a 4-chain ligand comprising (i) the common light chain paired with a first TCR-Ig heavy chain to form a first antigen binding site; and (ii) the common light chain paired with a second TCR-Ig heavy chain to form a second antigen binding site. In an alternative, there is provided a bispecific ligand comprising (i) and (ii) an antibody light chain paired with an antibody heavy chain and comprising a VH/VL antigen binding site, wherein the CL domains of (i) and (ii) are identical and the CH domains of (ii) are identical.

Example 3 herein demonstrates preferential use of certain TCR gene segments in a non-human vertebrate. Thus, in an example, the or a rearranged VDJ herein is the product of rearrangement of a V, D and J, wherein the V/J are selected from the group consisting of TCRBV27/TCRBJ1-5, TCRBV27/TCRBJ1-1, TCRBV20-1/TCRBJ1-5, TCRBV20-1/TCRBJ1-2, TCRBV20-1/TCRBJ1-4, TCRBV29-1/TCRBJ1-5, TCRBV28/TCRBJ1-5, TCRBV20-1/TCRBJ1-1, TCRBV27/TCRBJ1-2 and TCRBV29-1/TCRBJ1-4. In an alternative, the group consists of TCRBV27*01/TCRBJ1-5, TCRBV27*01/TCRBJ1-1, TCRBV20-1*02/TCRBJ1-5, TCRBV20-1*02/TCRBJ1-2, TCRBV20-1*01/TCRBJ1-4, TCRBV29-1*02/TCRBJ1-5, TCRBV28*01/TCRBJ1-5, TCRBV20-1*02/TCRBJ1-1, TCRBV27*01/TCRBJ1-2 and TCRBV29-1*01/TCRBJ1-4. In an alternative, the group consists of TCRBV27*01/TCRBJ1-5*01, TCRBV27*01/TCRBJ1-1*01, TCRBV20-1*02/TCRBJ1-5*01, TCRBV20-1*02/TCRBJ1-2*01, TCRBV20-1*01/TCRBJ1-4*01, TCRBV29-1*02/TCRBJ1-5*01, TCRBV28*01/TCRBJ1-5*01, TCRBV20-1*02/TCRBJ1-1*01, TCRBV27*01/TCRBJ1-2*01 and TCRBV29-1*01/TCRBJ1-4*01. In an example, the or a rearranged VDJ herein is the product of rearrangement of a V, D and J, wherein the V/J is TCRBV27/TCRBJ1-5, eg, TCRBV27*01/TCRBJ1-5 or TCRBV27*01/TCRBJ1-5*01.

In an example, the vertebrate expresses a plurality of different rearranged TCR VDJ, wherein each VDJ is the product of rearrangement of a V, D and J, wherein the V/J are selected from the group consisting of TCRBV27/TCRBJ1-5, TCRBV27/TCRBJ1-1, TCRBV20-1/TCRBJ1-5, TCRBV20-1/TCRBJ1-2, TCRBV20-1/TCRBJ1-4, TCRBV29-1/TCRBJ1-5, TCRBV28/TCRBJ1-5, TCRBV20-1/TCRBJ1-1, TCRBV27/TCRBJ1-2 and TCRBV29-1/TCRBJ1-4. In an alternative, the group consists of TCRBV27*01/TCRBJ1-5, TCRBV27*01/TCRBJ1-1, TCRBV20-1*02/TCRBJ1-5, TCRBV20-1*02/TCRBJ1-2, TCRBV20-1*01/TCRBJ1-4, TCRBV29-1*02/TCRBJ1-5, TCRBV28*01/TCRBJ1-5, TCRBV20-1*02/TCRBJ1-1, TCRBV27*01/TCRBJ1-2 and TCRBV29-1*01/TCRBJ1-4. In an alternative, the group consists of TCRBV27*01/TCRBJ1-5*01, TCRBV27*01/TCRBJ1-1*01, TCRBV20-1*02/TCRBJ1-5*01, TCRBV20-1*02/TCRBJ1-2*01, TCRBV20-1*01/TCRBJ1-4*01, TCRBV29-1*02/TCRBJ1-5*01, TCRBV28*01/TCRBJ1-5*01, TCRBV20-1*02/TCRBJ1-1*01, TCRBV27*01/TCRBJ1-2*01 and TCRBV29-1*01/TCRBJ1-4*01. In an example, the plurality comprises one or more rearranged VDJ, wherein each VDJ is the product of rearrangement of a V, D and J, wherein the V/J is TCRBV27/TCRBJ1-5, eg, TCRBV27*01/TCRBJ1-5 or TCRBV27*01/TCRBJ1-5*01.

In an example, the cell expresses a rearranged TCR VDJ which is the product of rearrangement of a V, D and J, wherein the V/J are selected from the group consisting of TCRBV27/TCRBJ1-5, TCRBV27/TCRBJ1-1, TCRBV20-1/TCRBJ1-5, TCRBV20-1/TCRBJ1-2, TCRBV20-1/TCRBJ1-4, TCRBV29-1/TCRBJ1-5, TCRBV28/TCRBJ1-5, TCRBV20-1/TCRBJ1-1, TCRBV27/TCRBJ1-2 and TCRBV29-1/TCRBJ1-4. In an alternative, the group consists of TCRBV27*01/TCRBJ1-5, TCRBV27*01/TCRBJ1-1, TCRBV20-1*02/TCRBJ1-5, TCRBV20-1*02/TCRBJ1-2, TCRBV20-1*01/TCRBJ1-4, TCRBV29-1*02/TCRBJ1-5, TCRBV28*01/TCRBJ1-5, TCRBV20-1*02/TCRBJ1-1, TCRBV27*01/TCRBJ1-2 and TCRBV29-1*01/TCRBJ1-4. In an alternative, the group consists of TCRBV27*01/TCRBJ1-5*01, TCRBV27*01/TCRBJ1-1*01, TCRBV20-1*02/TCRBJ1-5*01, TCRBV20-1*02/TCRBJ1-2*01, TCRBV20-1*01/TCRBJ1-4*01, TCRBV29-1*02/TCRBJ1-5*01, TCRBV28*01/TCRBJ1-5*01, TCRBV20-1*02/TCRBJ1-1*01, TCRBV27*01/TCRBJ1-2*01 and TCRBV29-1*01/TCRBJ1-4*01. In an example, the VDJ is the product of rearrangement of a V, D and J, wherein the V/J is TCRBV27/TCRBJ1-5, eg, TCRBV27*01/TCRBJ1-5 or TCRBV27*01/TCRBJ1-5*01.

In an example, the locus comprises a human, mouse or rat antibody locus intronic enhancer (eg, a Eμ or iEκ enhancer) between the variable and constant regions and/or a human, mouse or rat antibody locus 3′ enhancer operably linked downstream of said constant region. For example, the enhancer is a mouse Eμ and the constant region is an antibody heavy chain constant region. For example, the enhancer is a mouse iEκ and the constant region is an antibody kappa chain constant region. For example the 3′ enhancer is a mouse antibody heavy chain locus 3′ enhancer. For example the 3′ enhancer is a mouse antibody kappa chain locus 3′ enhancer.

The constant region optionally comprises the endogenous antibody heavy chain locus Eμ and Cμ of the vertebrate, optionally wherein the constant region comprises the DNA sequence of the endogenous Eμ through to (and including) the Cμ of the vertebrate.

The constant region optionally comprises the endogenous antibody heavy chain locus mu switch sequence (Sμ) of the vertebrate, the constant region comprising downstream of the Cμ a second switch sequence and a second C segment, wherein the constant region is capable of class-switch recombination (CSR) between the switches for isotype switching from the Cμ to the second constant region gene segment and somatic hypermutation (SHM) of the TCR variable region. SHM is useful to produce a plurality of affinity matured TCR V domains, for example comprising antigen-binding affinities that are stronger than typically found for natural TCR binding sites and V domains.

The constant region optionally comprises
a. a first antibody C segment (eg, a Cμ) operably linked to a first switch sequence (eg, a Sμ);
b. a second antibody C segment operably linked to a second switch sequence;
c. wherein the constant region is capable of CSR between the switches for isotype switching from the first to the second C segment and SHM of the TCR variable region.

Operable linkage in this respect will be clear to the skilled person as involving the usual recombination between switch sequences to effect CSR and isotype switching, as seen in IgH loci. In an example, the second C segment is a human or a mouse gamma C, eg, gamma-1, gamma-2, gamma-3 or gamma-4 C segment. In an example, the segment is a mouse gamma-1 C, eg, an endogenous C when the vertebrate is a mouse or rat. In an example, the segment is a human gamma-1 C.

In an example, the genome of the vertebrate comprises an endogenous activation induced cytidine deaminase (AID) nucleotide sequence that is capable of expressing AID for SHM of the TCR variable region. The vertebrate genome may comprise a nucleotide sequence for expressing an endogenous RAG-1 and/or RAG-2.

A particularly useful example is a vertebrate of the that expresses paired TCR V domains that provide an antigen binding site, wherein the V domains are encoded by loci of the invention (eg, after rearrangement of the variable region and SHM following exposure of the vertebrate to the antigen). There is provided, therefore, in an embodiment a vertebrate of the invention comprising a first said locus and a second said locus, wherein
a. the TCR variable region of the first locus comprises one or more TCRAV segments and one or more TCRAJ gene segments and optionally the one or more antibody C gene segments are kappa C segments; and the TCR variable region of the second locus comprises one or more TCRBV segments, one or more TCRBD segments and one or more TCRBJ gene segments and optionally the one or more antibody C gene segments are heavy chain C segments, wherein the antigen binding site of each ligand comprises a TCR Vα domain and a TCR Vβ domain and optionally paired antibody heavy and kappa C domains; or
b. the TCR variable region of the first locus comprises one or more TCRGV segments and one or more TCRGJ gene segments and optionally the one or more antibody C gene segments are kappa C segments; and the TCR variable region of the second locus comprises one or more TCRDV segments, one or more TCRDD segments and one or more TCRDJ gene segments and optionally the one or more antibody C gene segments are heavy chain C segments, wherein the antigen binding site of each ligand comprises a TCR Vγ domain and a TCR Vδ domain and optionally paired antibody heavy and kappa C domains.

Thus, such ligands expressed from the vertebrate are a useful source of paired VαVβ or VγVδ antigen binding sites, or a source of the V domains per se; and/or a source of nucleotide sequences encoding these. For example, the invention contemplates isolating or copying such a nucleotide sequence and inserting it into an expression vector (eg, harboured by a host cell, such as a CHO or Hek293 or other cell) for expression of the cognate V domain. By inserting such a nucleotide sequence encoding a TCR Vα domain into the genome of the cell, and inserting a nucleotide sequence encoding a TCR Vβ into the genome, the cell can express a VαVβ paired antigen binding site; and the cell can be grown into a cell line for expressing such a binding site. By inserting such a nucleotide sequence encoding a TCR Vγ domain into the genome of the cell, and inserting a nucleotide sequence encoding a TCR Vδ into the genome, the cell can express a VγVδ paired antigen binding site; and the cell can be grown into a cell line for expressing such a binding site.

In an example, the V and J gene (and optional D) segments are human gene segments, optionally wherein the antibody C gene segments are human, rat or mouse gene segments. In an example, one or more or all of the V gene segments is synthetic, eg, each V is a mutated germline TCRV gene segment. In an example, one or more or all of the D gene segments is synthetic, eg, each D is a mutated germline TCRD gene segment. In an example, one or more or all of the J gene segments is synthetic, eg, each J is a mutated germline TCRJ gene segment.

The or each variable region is optionally not at an endogenous antibody locus. For example, the locus (or one or all of the loci) is a product of random insertion into the vertebrate genome. For example, the locus has been targeted into the genome, eg, the locus is at a Rosa 26 locus.

The vertebrate may be obtainable or obtained in a method by
a. providing an embryonic stem cell of the vertebrate species (eg, mouse or rat);
b. inserting DNA comprising said TCR variable region gene segments into the ES cell genome in one or several steps to produce an ES cell product whose genome comprises the inserted variable region DNA operably linked upstream of the antibody constant region for expression of said polypeptides (ie, in a vertebrate developed from the cell or a progeny thereof); and
c. developing said vertebrate from said product ES cell or a progeny thereof;
   wherein either
d. the TCR variable region DNA (eg, TCR beta variable region DNA) is inserted into an endogenous antibody locus (eg, a heavy chain locus) of the genome and the constant region comprises one or more C gene segments of the endogenous locus, wherein the insertion produces an engineered locus that is capable of expressing said polypeptides and CSR and SHM of the variable region; or
e. the TCR variable region DNA (eg, TCR beta variable region DNA) is comprised by a transgene, wherein the transgene comprises said constant region (eg, comprising a CH gene segment), wherein the transgene is inserted into said genome to provide a transgene locus that is capable of expressing said polypeptides and CSR and SHM of the variable region.

The step of inserting DNA in step (b) can be performed in one or multiple steps (depending, for example, upon the amount of DNA to be inserted) using standard techniques, eg, employing BACs and homologous recombination and/or site-specific recombination (eg, RMCE using cre-lox technology). The insertion may not concomitantly delete endogenous DNA or it may do so simultaneously or before or after the insertion. DNA insertion may be in several smaller parts using a plurality of ES cells (such as using standard techniques involving insertions into genomes of ES cells in a lineage). Optionally, it may be desirable to re-derive ES cells from mice or other vertebrates during the process, wherein the re-derived ES cells receive one or more further insertions of DNA. All of these techniques for building ES cell genomes by targeted insertion and developing mice, rats or other vertebrates from ES cells are conventional and known to the skilled person.

The step of developing the vertebrate from the product ES cell can also be performed conventionally by inserting the ES cell into a blastocysts and implanting a pseudopregnant mother. Chimaera progeny can be made and crossed to produce progeny mice which are according to the invention (eg comprising a homozygous locus according to the invention).

By providing TCR V gene segments at an endogenous antibody locus, the endogenous control of the locus can be harnessed (eg, for proper functioning of the locus in a B-cell to express TCR V domains).

Optionally, in the method the insertion in step (d) of TCR variable region DNA is an insertion (i) immediately 5′ of the 5′-end of the intron of said endogenous antibody locus; or (ii) between said 5′ end and the intronic enhancer (eg, Eμ) of the intron. The intron is the stretch of DNA naturally contiguous with and immediately 3′ of the last (3′-most) antibody J segment in an antibody locus to and including the nucleotide naturally immediately 5′ of the Cmu or CL.

Optionally, (i) the engineered locus comprises less than the complete intronic sequence immediately 5′ of said intronic enhancer found in wild-type vertebrates of said species; and/or (ii) the distance between the last inserted human J gene segment and said intronic enhancer is not >1 or 0.5 kb more (or no more) than the distance between the last antibody J gene segment and the enhancer found in wild-type vertebrates of said species; and/or (iii) the inserted DNA comprises a 3′-most TCR J gene segment, wherein the segment is immediately 5′ of a further nucleotide sequence, wherein the further sequence is intron sequence that is naturally contiguous (ie, in a wild-type respective TCR locus in the genome of a human or other species from which the inserted DNA is derived) with said TCR J segment and the further sequence is no more than 1 or 0.5 kb in length. In an example, the locus comprises complete intronic sequence immediately 5′ of said intronic enhancer found in wild-type vertebrates of said species, but with the omission of up to the first (ie, 5′-most) contiguous 1 kb, 900 bp, 800 bp, 700 bp, 600 bp, 500 bp, 400 bp, 300 bp, 200 bp or 100 bp of the intron found in said wild-type vertebrates. Thus, the locus comprises such a wild-type intronic sequence that is missing its first (5′-most) contiguous 1000-100 bps.

The invention also contemplates a non-human vertebrate that is a progeny of the vertebrate developed in step (c) of the method, wherein the progeny vertebrate is according to the invention.

In any example herein, the vertebrate may be incapable of antibody heavy chain and/or kappa chain variable region expression. The vertebrate may additionally or alternatively be incapable of antibody lambda chain expression. This may be achieved by deleting or disrupting one or more respective antibody loci or variable regions in the germline genome of the vertebrate (eg, by J region deletion, neo insertion into an endogenous V region and/or inversion of an endogenous V region). In an example, the vertebrate may be incapable of non-human vertebrate antibody heavy chain and/or kappa chain variable region expression. In alternative, the vertebrate is capable of expressing antibody heavy chains (eg, from a genomically-integrated transgene or an allele of an endogenous IgH locus, such as where the other allele is a locus according to the invention) and/or light chains (eg, from a genomically-integrated transgene or an allele of an endogenous IgL locus, such as where the other allele is a locus according to the invention).

In alternative, the vertebrate is capable of expressing antibody heavy chains (eg, from a genomically-integrated transgene or an allele of an endogenous IgH locus, such as where the other allele is a locus according to the invention) and/or light chains (eg, from a genomically-integrated transgene or an allele of an endogenous IgL locus, such as where the other allele is a locus according to the invention).

In an example, the germline genome of the vertebrate comprises one or more expressible ADAM6 nucleotide sequences (eg, mouse ADAM6a and/or ADAM6b, eg, wherein the vertebrate is a mouse), eg, in homozygous state. In an example, the vertebrate has wild-type fertility typical of wild-type vertebrates of the same species that comprise functional homozygous ADAM6 genes.

In an example, the vertebrate is incapable of non-human vertebrate (i) TCR Vβ domain and/or TCR Vα domain expression (eg, wherein the vertebrate is capable of expressing human TCR Vβ domain and/or TCR Vα domains); (ii) TCR Vδ domain and/or TCR Vγ domain expression (eg, wherein the vertebrate is capable of expressing human TCR Vδ domain and/or TCR Vγ domains); (iii) TCR Vβ and TCR Vδ domain expression (eg, wherein the vertebrate is capable of expressing human TCR Vβ and TCR Vδ domains); or (iv) TCR Vβ, Vα, Vδ and Vγ domain expression (eg, wherein the vertebrate is capable of expressing human TCR Vβ, Vα, Vδ and Vγ domains).

In an example, the vertebrate comprises antigen presenting cells comprising nucleic acid for surface expressing a peptide antigen receptor comprising a human MHC protein (eg, Class I or Class II MHC), wherein the vertebrate is capable of producing said plurality of ligands when the vertebrate is immunised with a peptide-MHC antigen (pMHC) comprising said human MHC protein. This is useful as the vertebrate expresses the MHC as self-protein, thereby focusing the immune response on the antigen with which the vertebrate is immunised. In an embodiment, the vertebrate additionally (when the MHC is class I MHC) or alternatively expresses human beta-2 microglobulin which is capable of forming peptide-presenting complex with the MHC in the vertebrate. In an embodiment, in these instances the vertebrate expresses human TCR Vβ domain and/or TCR Vα domains that form a binding site for said pMHC.

Thus, for example the vertebrate comprises antigen presenting cells that further comprise nucleic acid for surface expressing human beta-2 microglobulin complexed with the MHC protein, wherein the MHC protein is a human class I MHC protein, eg, HLA-A2. This is further useful for focussing the immune response just to the peptides, as the antigen receptor components will be seen as self in the vertebrate.

An aspect of the invention provides a non-human vertebrate embryo which is capable of developing into a vertebrate of the invention. In an embodiment the embryo or vertebrate of the invention is male. In an embodiment the embryo or vertebrate of the invention is female. In an embodiment the vertebrate of the invention is an adult. In an embodiment the vertebrate of the invention is an infant. In an embodiment the embryo or vertebrate of the invention is a chimaera of two or more genomes of said non-human vertebrate species (eg, two mouse strains).

An aspect of the invention provides an isolated ES cell, iPS cell, immune cell (eg, NK cell or TIL), B-cell; thymus cell (eg, T-cell) or tissue; spleen cell or tissue; or bone marrow cell or tissue obtainable or obtained from a vertebrate of the invention, eg, in a sterile container. Another aspect provides a plurality (eg, at least 10, 100, 1000, 10⁴, 10⁵, 10⁶, 10⁷, 10⁸, 10⁹, 10¹⁰ or 10¹¹) of said immune, B-, thymus, T-, spleen or bone marrow cells, eg, in a sterile container. The container may be an IV bag, syringe, test tube, flask or petri dish.

2.

An aspect of the invention provides:

A method of producing one or more polypeptides, wherein each polypeptide comprises a T-cell receptor (TCR) variable domain and an antibody constant domain, the method comprising
a. providing a vertebrate according to the invention;
b. immunising the vertebrate with an antigen to effect CSR and SHM in the vertebrate, whereby a plurality of polypeptides comprising affinity matured TCR V domains and antibody constant domains are expressed by B-cells of the vertebrate; and
c. selecting one or more B-cells capable of expressing a said polypeptide; selecting one or more of said polypeptides; isolating one or more affinity matured TCR V domains thereof; or isolating one or more nucleotide sequences each encoding an affinity matured TCR V domain of a said expressed polypeptide;
d. wherein the polypeptide(s) or TCR V domain(s) of (c) specifically binds to the antigen; and
e. optionally expressing said polypeptide(s) or TCR V domain(s).

In an example, the polypeptides in any configuration, aspect, example or embodiment herein are comprised by a TCR-Ig.

Standard immunisation protocols can be used, such as RIMMS, or a prime-boost protocol. In an embodiment, the antigen is a pMHC antigen. In an embodiment, the antigen is comprised by a cancer cell or viral- or bacterial-infected cell that surface-expresses the antigen, wherein the vertebrate is immunised with such a cell or with a membrane sample of such a cell. A particularly useful vertebrate for CSR and SHM is a vertebrate of the invention comprising an endogenous AID enzyme, endogenous Terminal deoxynucleotidyl transferase (TdT) and a constant region comprising (in 5′ to 3′ orientation) at least a Smu-Cmu (ie, mu switch and mu C segment) and a Sgamma-Cgamma. The Smu-Cmu may be endogenous. The Sgamma-Cgamma may be endogenous, mouse, rat or human.

The skilled person will know that an affinity matured TCR V domain will comprise mutations that are not encoded by germline VDJ or VJ sequences, for example, the V domain will be encoded by a sequence comprising junctional mutation (nucleotide addition, substitution and/or deletion) between (i) V and D; and/or D and J (for a VDJ TCR V domain); or between (iii) V and J (for a VJ TCR V domain). Germline sequences are found, for example, in the IMGT Repertoire database (see http://www.imgt.org/IMGTrepertoire/LocusGenes/#h1_6).

Conventional techniques for screening and selecting antigen binding sites can be used in the methods herein, as will be readily apparent to the skilled person.

The isolated nucleotide sequences in any configuration, aspect, example or embodiment herein may be comprised by DNA, cDNA, RNA or mRNA.

The TCR V domain in any configuration, aspect, example or embodiment herein may bind to the antigen without the need for a partner V domain (akin to a dAb); or in another embodiment, the TCR

V domain may be comprised by an antigen binding site wherein the domain is paired with another TCR V domain.

A V domain or binding site that “specifically binds to” or is “specific for” a particular antigen or epitope is one that binds to that particular antigen or epitope without substantially binding to other antigens or epitopes. For example, binding to the antigen or epitope is specific when the antibody binds with a K_{D} of 1 mM or less, eg, 100 μM or less, 10 μM or less, 1 μM or less, 100 nM or less, eg, 10 nM or less, 1 nM or less, 500 μM or less, 100 μM or less, or 10 μM or less. The binding affinity (K_{D}) can be determined using standard procedures as will be known by the skilled person, eg, binding in ELISA and/or affinity determination using surface plasmon resonance (eg, Biacore™, Proteon™ or KinExA™ solution phase affinity measurement which can detect down to fM affinities (Sapidyne Instruments, Idaho)). In one embodiment, the surface plasmon resonance (SPR) is carried out at 25° C. In another embodiment, the SPR is carried out at 37° C. In one embodiment, the SPR is carried out at physiological pH, such as about pH7 or at pH7.6 (eg, using Hepes buffered saline at pH7.6 (also referred to as HBS-EP)). In one embodiment, the SPR is carried out at a physiological salt level, eg, 150 mM NaCl. In one embodiment, the SPR is carried out at a detergent level of no greater than 0.05% by volume, eg, in the presence of P20 (polysorbate 20; eg, Tween-20TM) at 0.05% and EDTA at 3 mM. In one example, the SPR is carried out at 25° C. or 37° C. in a buffer at pH7.6, 150 mM NaCl, 0.05% detergent (eg, P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022).

In an example, the affinity is determined using SPR by

1. Coupling anti-mouse (or other relevant non-human vertebrate, to match the C region of a TCR-Ig for example) IgG (eg, Biacore BR-1008-38) to a biosensor chip (eg, GLM chip) such as by primary amine coupling;
2. Exposing the anti-mouse IgG (non-human vertebrate antibody) to a test IgG antibody or heavy chain to capture test antibody on the chip;
3. Passing the test antigen over the chip's capture surface at 1024 nM, 256 nM, 64 nM, 16 nM, 4 nM with a 0 nM (i.e. buffer alone); and
4. And determining the affinity of binding of test antibody/chain to test antigen using surface plasmon resonance, eg, under an SPR condition discussed above (eg, at 25° C. in physiological buffer). SPR can be carried out using any standard SPR apparatus, such as by Biacore™ or using the ProteOn XPR36™ (Bio-Rad®).

Regeneration of the capture surface can be carried out with 10 mM glycine at pH1.7. This removes the captured antibody and allows the surface to be used for another interaction. The binding data can be fitted to 1:1 model inherent using standard techniques, eg, using a model inherent to the ProteOn XPR36™ analysis software.

In an example of the method of the invention, each polypeptide is comprised an antigen-specific ligand, wherein the ligand comprises an antigen binding site, wherein the binding site comprises the TCR V domain of the polypeptide, the method optionally comprising selecting one or more B-cells capable of expressing a said ligand; selecting one or more of said ligands; or isolating one or more nucleic acid sequences each encoding a said expressed ligand or the binding site thereof; wherein the ligand specifically binds to said antigen used in step (b).

An aspect of the invention provides the following method which is useful for harnessing in vivo systems to select for properly folded and expressed matured TCR V domains and their nucleotide sequences:

A method of using a non-human vertebrate (eg, according to any configuration, aspect, example or embodiment herein) to select for an affinity matured TCR variable domain or a nucleotide sequence encoding an affinity matured TCR variable domain, wherein the variable domain is capable of expression in vivo in a vertebrate, the method comprising
a. providing a non-human vertebrate wherein at least one antibody heavy chain locus of the vertebrate comprises a first TCR variable region upstream of the antibody heavy chain constant region for expressing first mRNA transcripts encoding polypeptides comprising an affinity matured TCR V domain and an antibody C domain; at least one antibody light chain (eg, kappa or lambda) locus of the vertebrate comprises a second TCR variable region upstream of the antibody light chain locus for expressing second mRNA transcripts encoding polypeptides comprising a TCR V domain and an antibody C domain;
b. immunising the vertebrate with an antigen to effect CSR and SHM in the vertebrate, whereby a plurality of antigen-specific TCR-Ig ligands comprising affinity matured TCR V domains are expressed by B-cells of the vertebrate; and
c. selecting one or more B-cells capable of expressing a said TCR-Ig; selecting one or more of said TCR-Ig; isolating one or more affinity matured TCR V domains thereof; or isolating one or more nucleic acid sequences (eg, mRNA or DNA) each encoding an affinity matured TCR V domain of an expressed TCR-Ig;
d. wherein the TCR-Ig(s) or TCR V domain(s) of (c) specifically bind to the antigen; and
e. Optionally expressing the one or more TCR-Igs or one or more TCR V domains.

The antigen can be pMHC antigen or any other antigen disclosed herein.

Thus when the vertebrate is a mouse, in one aspect it is expected that the mouse can for example express one, more or all of:—

(i) serum TCR-Ig at a concentration of 25-350 μg/ml, when the TCR-Ig comprises an IgG1 constant region;
(ii) serum TCR-Ig at a concentration of 1-200 μg/ml μg/ml, when the TCR-Ig comprises an IgG2a constant region;
(iii) serum TCR-Ig at a concentration of 30-800 μg/ml, when the TCR-Ig comprises an IgG2b constant region; and
(iv) serum TCR-Ig at a concentration of 50-300 μg/ml, when the TCR-Ig comprises an IgGM constant region;
or all of
(i) serum TCR-IgG1 at a concentration of 10-600 μg/ml;
(ii) serum TCR-IgG2a at a concentration of 0-500 μg/ml;
(iii) serum TCR-IgG2b at a concentration of 20-700 μg/ml; and
(iv) serum TCR-IgM at a concentration of 50-700 μg/ml.

Expression is determined by Ig capture on a plate followed by incubation (eg, for one hour at RT, eg, for one hour at 20° C.) with anti-mouse isotype-specific labelled antibodies and quantification of Ig using the label (eg, using anti-mouse Ig isotype specific antibodies each conjugated to horseradish peroxidase conjugated at a ratio of 1/10000 in PBS with 0.1% Tween™, followed by development of the label with tetramethylbenzidine substrate (TMB) for 4-5 minutes in the dark at room temperature (eg, 20° C.), adding sulfuric acid to stop development of the label and reading of the label at 450 nm).

In an example, the mouse comprises a mouse constant region, wherein the constant region comprises mu, gamma-1, gamma-2a and gamma-2b C gene segments. Optionally, the TCR-Ig has the format of a 4-chain antibody except with TCR V domains instead of antibody V domains. Additionally or alternatively, the mouse is incapable of expressing 4-chain antibodies.

In an example, the mouse expresses serum TCR-Ig at a concentration of 25-350 μg/ml, when the TCR-Ig comprises an IgG1 constant region.

In an example, the mouse expresses serum TCR-IgG1 at a concentration of 10-600 μg/ml

Thus, in one aspect it is expected that the mouse according to the invention can for example express

(i) serum TCR-IgG1 at a concentration of about 25-150 μg/ml;
(ii) serum TCR-IgG2a at a concentration of about 0-200 μg/ml;
(iii) serum TCR-IgG2b at a concentration of about 30-300 μg/ml; and
(iv) serum TCR-IgM at a concentration of about 50-200 μg/ml;
or
(i) serum TCR-IgG1 at a concentration of about 10-200 μg/ml;
(ii) serum TCR-IgG2a at a concentration of about 0-500 μg/ml;
(iii) serum TCR-IgG2b at a concentration of about 20-400 μg/ml; and
(iv) serum TCR-IgM at a concentration of about 50-700 μg/ml;
as determined by Ig capture on a plate followed by incubation (eg, for one hour at RT, eg, for one hour at 20° C.) with anti-mouse isotype-specific labelled antibodies and quantification of Ig using the label (eg, using anti-mouse Ig isotype specific antibodies each conjugated to horseradish peroxidase conjugated at a ratio of 1/10000 in PBS with 0.1% Tween™, followed by development of the label with tetramethylbenzidine substrate (TMB) for 4-5 minutes in the dark at room temperature (eg, 20° C.), adding sulfuric acid to stop development of the label and reading of the label at 450 nm); wherein
the mouse comprises a mouse constant region, wherein the constant region comprises mu, gamma-1, gamma-2a and gamma-2b C gene segments; and optionally wherein the TCR-Ig has the format of a 4-chain antibody except with TCR V domains instead of antibody V domains.

Thus, in one aspect it is expected that the mouse according to the invention can for example express Ig in the relative proportions of

(i) serum TCR-IgG1 at a concentration of about 25-150 μg/ml;
(ii) serum TCR-IgG2a at a concentration of about 0-200 μg/ml;
(iii) serum TCR-IgG2b at a concentration of about 30-300 μg/ml; and
(iv) serum TCR-IgM at a concentration of about 50-200 μg/ml;
or
(i) serum TCR-IgG1 at a concentration of about 10-200 μg/ml;
(ii) serum TCR-IgG2a at a concentration of about 0-500 μg/ml;
(iii) serum TCR-IgG2b at a concentration of about 20-400 μg/ml; and
(iv) serum TCR-IgM at a concentration of about 50-700 μg/ml;
as determined by Ig capture on a plate followed by incubation (eg, for one hour at RT, eg, for one hour at 20° C.) with anti-mouse isotype-specific labelled antibodies and quantification of Ig using the label (eg, using anti-mouse Ig isotype specific antibodies each conjugated to horseradish peroxidase conjugated at a ratio of 1/10000 in PBS with 0.1% Tween™, followed by development of the label with tetramethylbenzidine substrate (TMB) for 4-5 minutes in the dark at room temperature (eg, 20° C.), adding sulfuric acid to stop development of the label and reading of the label at 450 nm); wherein
the mouse comprises a mouse constant region, wherein the constant region comprises mu, gamma-1, gamma-2a and gamma-2b C gene segments; and optionally wherein the TCR-Ig has the format of a 4-chain antibody except with TCR V domains instead of antibody V domains.

Thus, in one aspect it is expected that mice according to the invention can for example express heavy chains (ie, polypeptides comprising TCR V domains and antibody heavy chain constant domains) from splenic B-cells in a mouse that produces a normal proportion or percentage of mature splenic B-cells, eg as determined by FACS. By “normal” is meant comparable to mature splenic B-cell production in a mouse (eg, a naïve mouse) expressing only mouse antibody chains, eg, a mouse whose genome comprises only wild-type functional Ig heavy and light chain loci, eg, a wild-type mouse.

For example, at least 40, 50, 60 or 70% of total splenic B-cells produced by the mouse of the invention are mature B-cells. Splenic B-cells are B220⁺ and express B220 at relatively high levels as the skilled person will know. Mature splenic B-cells express B220 and IgD, both at relatively high levels as will be known by the skilled person. IgM expression is relatively low in mature splenic B-cells, again as is known in the art. For example, see J Exp Med. 1999 Jul. 5; 190(1):75-89; “B cell development in the spleen takes place in discrete steps and is determined by the quality of B cell receptor-derived signals”; Loder F et al.

Optionally the mouse produces a normal ratio of T1, T2 and mature splenic B-cells, eg, as determined by FACS. For example, the mouse of the invention produces about 40-70% mature splenic B-cells, 15-35% splenic T1 cells; and 5-10% splenic T2 cells (percentage with reference to the total splenic B220-positive (high) population). For example, about 40-60% mature splenic B-cells, 15-30% splenic T1 cells; and 5-10% splenic T2 cells. By “normal” is meant comparable to a T1/T2/mature splenic B-cell proportion in a mouse (eg, a naïve mouse) expressing only mouse antibody chains, eg, a mouse whose genome comprises only wild-type functional Ig heavy and light chain loci, eg, a wild-type mouse.

Thus, in one aspect it is expected that mice according to the invention can for example express heavy chains (ie, polypeptides comprising TCR V domains and antibody heavy chain constant domains) in a mouse that produces a normal proportion or percentage of bone marrow B-cell progenitor cells (eg as determined by FACS).

In one embodiment, the mouse is for expressing said heavy chains in a mouse that produces a normal proportion or percentage of bone marrow pre-, pro and prepro-B-cells (eg as determined by FACS). See J Exp Med. 1991 May 1; 173(5):1213-25; “Resolution and characterization of pro-B and pre-pro-B cell stages in normal mouse bone marrow”; Hardy R R et al for more discussion on progenitor cells. By “normal” is meant comparable to bone marrow B-cell production in a mouse (eg, a naïve mouse) expressing only mouse antibody chains, eg, a mouse whose genome comprises only wild-type functional Ig heavy and light chain loci, eg, a wild-type mouse.

Optionally, in the method of the invention, for the first and second TCR variable regions,
a. one of said regions (eg, the first) is a TCRβ variable region and the other is a TCRα variable region;
b. one of said regions (eg, the first) is a TCRδ variable region and the other is a TCRγ variable region;
c. one of said regions (eg, the first) is a TCRβ variable region and the other is a TCRγ variable region;
d. one of said regions (eg, the first) is a TCRδ variable region and the other is a TCRα variable region.

In an example of the method, the vertebrate comprises a nucleotide sequence encoding a human MHC (as discussed above), wherein the vertebrate is immunised in step (b) with a pMHC antigen comprising said human MHC protein. This is useful for focussing the immune response just to the peptides, as the antigen receptor components will be seen as self in the vertebrate.

Optionally in the method, the constant region of the locus comprises a non-human (eg, rodent, mouse or rat) C segment and said polypeptides comprise an affinity matured human TCR V domain and a non-human C domain, the method further comprising isolating a nucleotide sequence encoding the variable domain and combining the sequence with a nucleotide sequence encoding a human antibody or TCR constant domain (eg, a human antibody Fc region) to produce an engineered nucleotide sequence capable of expressing a polypeptide comprising the human TCR V domain and the human C domain (eg, Fc region); and optionally expressing the polypeptide comprising the human TCR V domain and the human C domain. For example, the polypeptide comprises a TCR V beta with an antibody heavy chain constant region (ie, CH1-optional hinge-Fc); or a TCR V alpha with an antibody CL. In an embodiment, the method comprises expressing both of these, thereby producing a dimer of an antigen-binding ligand comprising a TCRVα/TCRVβ antigen binding site joined to a CH1 paired with the CL. For example, the polypeptide comprises a TCR V delta with an antibody heavy chain constant region (ie, CH1-optional hinge-Fc); or a TCR V gamma with an antibody CL. In an embodiment, the method comprises expressing both of these, thereby producing a dimer of an antigen-binding ligand comprising a TCRVy/TCRVδ antigen binding site joined to a CH1 paired with the CL.

In an embodiment, the or each polypeptide, ligand or dimer is capable of binding to a peptide-MHC expressed on tumour cells and engaging CDC, ADCC or ADCP to kill the tumour cells.

The invention further provides a composition (eg, a pharmaceutical composition or a composition for medical use) comprising a ligand, polypeptide, TCR-Ig, TCRV-Ig or TCR V domain or nucleotide sequence thereof disclosed herein; optionally wherein the composition comprises a diluent, excipient or carrier, optionally wherein the composition is contained in an IV container (eg, and IV bag) or a container connected to an IV syringe. When the composition is a pharmaceutical composition or a composition for medical use, the diluent, excipient or carrier is pharmaceutically acceptable. “Pharmaceutically acceptable” refers to approved or approvable by a regulatory agency of the USA Federal or a state government or listed in the U.S. Pharmacopeia or other generally recognized pharmacopeia for use in animals, including humans. A “pharmaceutically acceptable carrier, excipient, or adjuvant” refers to an carrier, excipient, or adjuvant that can be administered to a subject, together with an agent, e.g., any antibody or antibody chain described herein, and which does not destroy the pharmacological activity thereof and is nontoxic when administered in doses sufficient to deliver a therapeutic amount of the agent. In an example, the composition is for treating and/or preventing a disease or condition in a human or animal subject that is mediated by an antigen, wherein a sequence of the antigen is presented as pMHC on immune cells (eg, APCs) of the subject. In an example, the composition is for treating and/or preventing a disease or condition in a human or animal subject that is indicated by an antigen, wherein a sequence of the antigen is presented as pMHC biomarker on immune cells (eg, APCs) of the subject. Such a biomarker is a correlate of the existence or stage or severity of the disease or condition.

In an example, the expressed polypeptide is comprised by an antigen-binding ligand comprising a first antigen binding site, wherein the binding site comprises the human TCR V domain; optionally wherein the binding site comprises a TCR Vα domain paired with a TCR Vβ domain, or a TCR Vγ domain paired with a TCR Vδ domain.

Optionally herein, said Vβ or Vδ domain is a said affinity matured TCR V domain.

Optionally herein the ligand or polypeptide comprises a first antigen or epitope binding site (comprising a TCR V domain of the invention, or a TCR V domain pair of the invention), and a second binding site, wherein the second binding site comprises a further TCR V domain pair or an antibody VH/VL pair and the first and second binding sites bind first and second antigens or epitopes which are different. For example, the epitopes are different epitopes of the same target antigen. In an example, the first site specificaly binds a tumour associated antigen (TAA) or pMHC; and the second site specifically binds CD3 or CD16. This is useful for bridging between a tumour cell and an effector T-cell for promoting T-cell mediated killing of a tumour in a human or animal subject suffering from a cancer (eg, a haematological cancer, such as ALL, AML, CLL or a leukaemia).

Optionally, the first antigen comprises a peptide-MHC antigen (eg, a cancer cell antigen), and the second antigen comprises a cell-surface antigen (eg, a T-cell surface antigen, eg, CD3 or CD16).

Herein, in an example the peptide of the pMHC is a TAA peptide.

In some embodiments, the tumour antigen is GD2-ganglioside, CD19, CD20, EPCAM, or CSPG4. Other suitable tumor antigens include, for example, p185 HER2/neu (erb-B1; Pisk et al., J. Exp. Med., 181:2109-2117 (1995)); epidermal growth factor receptor (EGFR) (Harris et al., Breast Cancer Res. Treat, 29: 1-2 (1994)); carcinoembryonic antigens (CEA) (Kwong et al., J. Natl. Cancer Inst., 85:982-990 (1995); carcinoma-associated mutated mucins (MUC-1 gene products; Jerome et al., J. Immunol., 151:1654-1662 (1993)); E7 and E6 proteins of human papillomavirus (Ressing et al., J. Immunol, 154:5934-5943 (1995)); prostate specific membrane antigen (PSMA Israeh, et al., Cancer Res., 54:1807-1811 (1994)); and idiotypic epitopes or antigens, for example, immunoglobulin idiotypes or T cell receptor idiotypes (Chen et al., J. Immunol., 153: 4775-4787 (1994)).

The invention provides:

A multispecific or multivalent antigen-binding ligand obtainable or obtained by the method of the invention. This ligand may comprise at least 2, 3, 4 or 5 antigen or epitope binding sites, at least one or two or which comprise a TCR V domain or TCR V pair as described herein. One or more of the additional binding sites can be provided by an antibody domain (eg, V domain, C or Fcab), TCR domain (V or C) or a non-Ig domain.

Examples of suitable additional binding domains are: an antibody variable domain (eg, a VL or a VH, an antibody single variable domain (domain antibody or dAb), a camelid VHH antibody single variable domain, a shark immunoglobulin single variable domain (NARV), a Nanobody™ or a camelised VH single variable domain); a T-cell receptor binding domain; an immunoglobulin superfamily domain; an agnathan variable lymphocyte receptor (J Immunol; 2010 Aug. 1; 185(3):1367-74; “Alternative adaptive immunity in jawless vertebrates; Herrin B R & Cooper M D.); a fibronectin domain (eg, an Adnectin™); an antibody constant domain (eg, a CH3 domain, eg, a CH2 and/or CH3 of an Fcab™) wherein the constant domain is not a functional CH1 domain (defined as a CH1 domain that can associate with a light chain); an scFv; an (scFv)₂; an sc-diabody; an scFab; a centyrin and an epitope binding domain derived from a scaffold selected from CTLA-4 (Evibody™); a lipocalin domain; Protein A such as Z-domain of Protein A (eg, an Affibody™ or SpA); an A-domain (eg, an Avimer™ or Maxibod™); a heat shock protein (such as and epitope binding domain derived from GroEI and GroES); a transferrin domain (eg, a trans-body); ankyrin repeat protein (eg, a DARPin™); peptide aptamer; C-type lectin domain (eg, Tetranectin™); human γ-crystallin or human ubiquitin (an affilin); a PDZ domain; scorpion toxin; and a kunitz type domain of a human protease inhibitor. Further sources are variable domains and VH/VL pairs of antibodies disclosed in WO2007024715 at page 40, line 23 to page 43, line 23. This specific disclosure is incorporated herein by reference as though explicitly written herein to provide basis for antigen or epitope binding domains for use in the present invention and for possible inclusion in claims herein.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain.

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001)

Lipocalins are a family of extracellular proteins which transport small hydrophobic molecules such as steroids, bilins, retinoids and lipids. They have a rigid β-sheet secondary structure with a numer of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633 An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1.

Avimers™ are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins™) are derived from ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomising residues in the first α-helix and a β-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins™ consist of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U56818418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include upto 25 amino acids without affecting the overall fold of the microprotein. For further details of engineered knottin domains, see WO2008098796.

Other epitope binding domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7-Non-Antibody Scaffolds from Handbook of Therapeutic Antibodies (2007, edited by Stefan Dubel) and Protein Science 15:14-27 (2006). Epitope binding domains used in the present invention could be derived from any of these alternative protein domains.

In one embodiment of the invention a or each domain or antigen-binding site binds to antigen with a KD of 1 mM, for example a KD of 10 nM, 1 nM, 500 μM, 200 μM, 100 μM or 10 μM or less (ie, better affinity) to each antigen as measured by Biacore™ or Proteon™, such as the Biacore™ method as described in method 4 or 5 of WO2010136485 or as described elsewhere herein.

An aspect of the invention provides the novel configuration as follows:

A plurality of B-cells or hybridoma cells that express (eg, secrete) a plurality of different affinity matured TCR variable domains, wherein one or more of the variable domains specifically binds to an antigen.

B-cells do not normally express or secrete TCR V domains, thus, this and other aspects of the invention involving TCR expression in B-cells is non-natural (ie, not a natural phenomenon). Similarly, affinity maturation (eg, as mediated by AID and/or TdT at an antibody locus) of TCR V sequences is a novel phenomenon, and thus also are resultant affinity matured TCR V domains with a binding domains with a binding affinity (KD) of less than 100 or 50 nM (eg, 100 or 10 pM or less) as determined by surface plasmon resonance (SPR).

B-cell hybridomas usually express and secrete antibody V domains, not TCR V domains, so hybridomas secreting TCR V domains are, we believe, new in the art.

In an example, said plurality comprises at least 10, 100, 1000, 10⁴, 10⁵, 10⁶, 10⁷, 10⁸, 10⁹, 10¹⁰ or 10¹¹ of said B-cells or hybridoma cells. In an example, the plurality of B-cells is comprised by spleen or bone marrow tissue; or comprised by a vertebrate or embryo as described herein.

Optionally, said B-cells are rodent, mouse cells or rat cells. Optionally the cells are comprised by a sterile container, eg, any container disclosed herein.

Another aspect provides the novel configuration: A plurality of mammalian cells that express a plurality of at least 10 different affinity matured TCR variable domains, wherein one or more of the TCR variable domains specifically binds to an antigen.

Affinity matured TCR V domains are discussed above and may comprise one or more mutations (compared to germline TCR VDJ or VJ sequence) whereby each domain comprises gene segment junctional diversity and/or somatic diversity.

In an example, the plurality comprises at least 10, 100, 1000, 10⁴, 10⁵, 10⁶, 10⁷, 10⁸, 10⁹, 10¹⁰ or 10¹¹ different TCR VDJ and/or VJ combinations.

The mammalian cells of the invention are optionally mouse cells or human (eg, CHO or Hek293) cells.

In the cells of the invention for example, each variable domain is
a. a Vα domain secreted from a respective cell and encoded by an affinity matured VJ sequence comprised by the cell, wherein the VJ sequence is derived from the recombination of a TCRAV gene segment and a TCRAJ gene segment, wherein the plurality of cells comprises a plurality of VαJα sequences derived from different (eg, at least 3, 4, 5, 6, 7, 8, 9, 10, 11, 12, 13, 14, 15, 16, 17, 18, 19, 20, 30 or 40 different) TCRAV and/or TCRAJ gene segments;
b. a Vβ domain secreted from a respective cell and encoded by an affinity matured VDJ sequence comprised by the cell, wherein the VDJ sequence is derived from the recombination of a TCRBV gene segment, a TCRBD gene segment and a TCRBJ gene segment, wherein the plurality of cells comprises a plurality of VβDβJβ sequences derived from different (eg, at least 3, 4, 5, 6, 7, 8, 9, 10, 11, 12, 13, 14, 15, 16, 17, 18, 19, 20, 30 or 40 different) TCR TCRBV and/or TCRBJ gene segments;
c. a Vγ domain secreted from a respective cell and encoded by an affinity matured VJ sequence comprised by the cell, wherein the VJ sequence is derived from the recombination of a TCRGV gene segment and a TCRGV gene segment, wherein the plurality of cells comprises a plurality of VγJγ sequences derived from different (eg, at least 3, 4, 5, 6, 7, 8, 9, 10, 11, 12, 13, 14, 15, 16, 17, 18, 19, 20, 30 or 40 different) TCRGV and/or TCRGJ gene segments; or
d. a Vδ domain secreted from a respective cell and encoded by an affinity matured VDJ sequence comprised by the cell, wherein the VDJ sequence is derived from the recombination of a TCRDV gene segment, a TCRDD gene segment and a TCRDJ gene segment, wherein the plurality of cells comprises a plurality of VδDδJδ sequences derived from different (eg, at least 3, 4, 5, 6, 7, 8, 9, 10, 11, 12, 13, 14, 15, 16, 17, 18, 19, 20, 30 or 40 different) TCRDV and/or TCRDJ gene segments.

In the cells of the invention for example, each variable domain is
a. a Vα domain secreted from a respective cell and encoded by an affinity matured VJ sequence comprised by the cell, wherein the VJ sequence is derived from the recombination of a TCRAV gene segment and a TCRAJ gene segment, wherein the plurality of cells comprises a plurality (eg, at least 3, 4, 5, 6, 7, 8, 9, 10, 11, 12, 13, 14, 15, 16, 17, 18, 19, 20, 30 or 40 different) of VαJα sequences comprising different recombined gene segment junctional diversity;
b. a Vβ domain secreted from a respective cell and encoded by an affinity matured VDJ sequence comprised by the cell, wherein the VDJ sequence is derived from the recombination of a TCRBV gene segment, a TCRBD gene segment and a TCRBJ gene segment, wherein the plurality of cells comprises a plurality (eg, at least 3, 4, 5, 6, 7, 8, 9, 10, 11, 12, 13, 14, 15, 16, 17, 18, 19, 20, 30 or 40 different) of VβDβJβ sequences comprising different recombined gene segment junctional diversity;
c. a Vγ domain secreted from a respective cell and encoded by an affinity matured VJ sequence comprised by the cell, wherein the VJ sequence is derived from the recombination of a TCRGV gene segment and a TCRGV gene segment, wherein the plurality of cells comprises a plurality (eg, at least 3, 4, 5, 6, 7, 8, 9, 10, 11, 12, 13, 14, 15, 16, 17, 18, 19, 20, 30 or 40 different) of VγJγ sequences comprising different recombined gene segment junctional diversity; or
d. a Vδ domain secreted from a respective cell and encoded by an affinity matured VDJ sequence comprised by the cell, wherein the VDJ sequence is derived from the recombination of a TCRDV gene segment, a TCRDD gene segment and a TCRDJ gene segment, wherein the plurality of cells comprises a plurality (eg, at least 3, 4, 5, 6, 7, 8, 9, 10, 11, 12, 13, 14, 15, 16, 17, 18, 19, 20, 30 or 40 different) of VδDδJδ sequences comprising different recombined gene segment junctional diversity.

In the cells of the invention for example, each variable domain is joined to an antibody constant domain (eg, an antibody Fc or antibody CL, eg, Cκ or Cλ) or a TCR constant domain (eg, a TCR Ca or a TCR Cβ). For example, the cells express (i) TCR Vα domains joined directly or indirectly to a TCR Ca domains and/or (ii) TCR Vβ domains joined directly or indirectly to a TCR C βdomains.

In the cells of the invention for example, the cells secrete ligands wherein each ligand comprises a polypeptide comprising a said TCR variable domain and an antibody or TCR constant domain, the ligand comprising an antigen binding site wherein the binding site comprises the variable domain.

Optionally, each ligand is any ligand disclosed herein, eg, a multispecific ligand.

Optionally any ligand disclosed herein has the structure of an antibody (4-chain or H2 antibody) except wherein the ligand comprises an affinity matured TCR variable domain instead of an antibody variable domain (optionally, wherein the ligand comprises only TCR variable domains instead of antibody variable domains), or wherein the polypeptide or V domain is comprised by such a ligand.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., dAb, Fab, F(ab′)2, and Fv). The term “antibody” also includes H2 antibodies that comprise a dimer of a heavy chain (5′-VH-(optional Hinge)-CH2-CH3-3′) and are devoid of a light chain (akin to naturalluy-occurring H2 antibodies; see, eg, Nature. 1993 Jun. 3; 363(6428):446-8; Naturally occurring antibodies devoid of light chains; Hamers-Casterman C, Atarhouch T, Muyldermans S, Robinson G, Hamers C, Songa E B, Bendahman N, Hamers R).

Optionally, the vertebrate is a rodent (eg, a mouse or a rat). Optionally, the mouse is a 129 or C57BL/6 strain mouse (eg, a hybrid 129 or hybrid C57BL/6 strain mouse).

The invention, for example, includes the step of replacing the antibody C region of a TCR-Ig (eg, a TCRV-Ig) as disclosed herein with a TCR C region or domain (eg, a human TCR constant region, optionally comprising a TCR transmembrane domain, and one or two cytoplasmic co-stimulation signalling domains), eg, to produce a chimaeric antigen receptor (CAR). The invention also provides a ligand comprising TCR V and C domains wherein the ligand is obtained or obtainable by the invention. In an embodiment, the TCR V and C domains are human. In an embodiment, the ligand can be (or is) expressed by T-cells (eg, human T-cells, eg, allogeneic or autologous T-cells of a human patient) for directing the T-cells to kill tumour cells in a human subject, wherein the tumour cells express peptide-MHC to which the ligand specifically binds via its TCR V domain(s). Thus, the invention provides the ligand for this purpose. In an example, the T-cells are ex vivo; or in vivo.

An aspect of the invention provides:

A TCR-Ig (eg, a TCRV-Ig) comprising a TCR variable domain according to the invention, wherein the TCRV-Ig specifically binds to an antigen, eg, pMHC antigen.

For example, the TCR-Ig is obtained or obtainable by the method of the invention.

In an example, the TCR-Ig comprises (in N- to C-terminal direction) a TCR V domain directly fused to an antibody constant domain (eg, the N-terminal C domain of an antibody Fc region), wherein there is no TCR or antibody domain between the TCR V domain and antibody constant domain. In an embodiment, said TCR V domain is directly fused to a human antibody constant domain or region (eg, the N-terminal C domain of an antibody Fc region).

The invention further provides:

A TCR variable domain according to the invention, wherein the TCR V domain specifically binds to an antigen, eg, pMHC antigen.

For example, the TCR V is obtained or obtainable by the method of the invention.

The TCR-Ig or TCR V domain may be isolated and non-naturally-occurring.

Optionally, the V domain is comprised by an antigen-specific ligand (eg, a TCRV-Ig according to the invention) for use in a method of treating or preventing a disease in a patient, the method comprising administering the ligand to the patient wherein the ligand specifically binds the antigen for antagonising the antigen or killing cells expressing the antigen whereby the disease or condition is treated.

The invention provides:

A method of treating or reducing the risk of a disease or condition in a human or animal patient, the method comprising administering an antigen-specific ligand to the patient, wherein the ligand comprises a TCR V domain as described herein, wherein the ligand specifically binds the antigen for antagonising the antigen or killing cells expressing the antigen whereby the disease or condition is treated.

Optionally, a disease or condition herein is a cancer; autoimmune disease or condition; inflammatory disease or condition; or viral infection.

Optionally, the TCR V domain is fused to an antibody gamma-1 constant region, wherein the gamma-1 constant region provides ADCC or CDC effector function for cell killing in the patient, wherein the patient comprises cells expressing the antigen and the cells are killed thereby treating the disease or condition, wherein the disease or condition is a cancer; autoimmune disease or condition;

inflammatory disease or condition; or viral infection.

Optionally, the TCR V domain is fused to an antibody gamma-2 constant region

Optionally, the TCR V domain is fused to an antibody gamma-3 constant region

Optionally, the TCR V domain is fused to an antibody gamma-4 constant region

An aspect of the invention provides:

A multispecific (eg, bi- or tri-specific) ligand comprising
a. a first binding site comprising a TCR Vα domain/TCR Vβ domain pair or a TCR Vγ domain/TCR Vδ domain pair, wherein one or both of the V domains is a TCR V domain according to the invention, wherein the first binding site specifically binds a pMHC antigen or TAA; and
b. a second binding site that specifically binds to a T-cell or NK cell surface antigen, eg, CD3 or CD16.

Further discussion of multispecific ligands above is also applicable to this aspect.

Each of the pMHC, TAA and cell surface antigen may be human, wherein the ligand is for treating or preventing a disease or condition in a human, eg, a cancer.

Optionally, the binding sites are linked by a linker wherein the linker is a (G₄S)ₙ linker wherein n=1-10 (eg, 3, 4 or 5); or wherein the linker comprises one or more antibody constant region domains (eg, a CH1/CL pair); or one or more TCR constant region domains (eg, a TCRα constant domain/TCRAβ constant domain pair). Optionally, the constant region domains are disulphide bonded together.

Optionally, the ligand is an ImmTac™.

Optionally, any multispecific ligand herein is a bispecific ligand. The term “bispecific ligand” means a ligand which comprises specificity for two target molecules, and includes formats such as DVD-Ig, mAb², FIT-Ig, mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig and zybody, except that at least one of the antigen binding domains of any of these formats is a TCR V domain of the invention. For a review of bispecific formats, see Spiess, C., et al., Mol. Immunol. (2015).

Optionally, the multispecific ligand is a DVD-Ig, mAb², FIT-Ig, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, minibody, knobs-in-holes ligand, knobs-in-holes ligand with common light chain, knobs-in-holes ligand with common light chain and charge pairs, charge pairs, ligand having charge pairs with common light chain; in each case except that at least one of the antigen binding domains of any of these formats is a TCR V domain of the invention.

An antigen mentioned herein may, for example, be selected from immune checkpoint inhibitors (such as PD-1, TIGIT, TIM-3, LAG-3 and VISTA, e.g. TIGIT, TIM-3 and LAG-3), immune modulators (such as BTLA, hHVEM, CSF1R, CCR4, CD39, CD40, CD73, CD96, CXCR2, CXCR4, CD200, GARP, SIRPa, CXCL9, CXCL10, CD155 and CD137, e.g. GARP, SIRPa, CXCR4, BTLA, hVEM and CSF1R) and immune activators (such as CD137, GITR, OX40, CD40, CXCR3 (e.g. agonistic anti-CXCR3 antibodies), CD3, ICOS (e.g. agonistic anti-ICOS antibodies), for example. ICOS, CD137, GITR and OX40).

In an example, the antigen comprises an epitope of a tumour-associated antigen (TAA) or an immune checkpoint target, for treating or preventing a cancer or an autoimmune disease or condition. For example the TCRV-Ig, TCR V domain or ligand is an immune checkpoint antagonist or agonist.

In an embodiment, the second binding site comprises an antibody VH/VL pair (eg, an scFv). Alternatively, the second binding site comprises a second TCR V domain pair (eg, a second TCR Vα domain/TCR Vβ domain pair or a TCR Vγ domain/TCR Vδ domain pair).

Optionally, the ligand or the TCR V domain is for use in (or is used in) a method of adoptive T-cell transfer (ACT), comprising administering engineered T-cells to the patient, wherein the T-cells surface-express the TCR V domain. For example, this is for treating or reducing the risk of a cancer or cancer relapse or cancer progression in a human or animal subject.

The binding affinity of natural TCR-antigen (eg, pMHC) interactions is around KD˜0.1-500 μM. In an example, the KD for binding of the TCR V or TCRV-Ig or ligand to the antigen is less than 100 nM, 10 nM, 1 nM, 100 pM or 10 pM, eg, 1 nM KD≤90 nM, eg, from 50 nM to 95, 90, 85 or 80 nM. Affinities lower than 100 nM are useful to promote preferential binding to the engineered protein (eg CAR) comprising the TCR V domain or TCRV-Ig rather than endogenous TCR binding on the surface of immune cells in a patient.

The TCRV-Ig, TCR V domain or ligand, thus in an embodiment, binds said antigen (eg, pMHC) with a binding affinity (KD) of less than 100, 90, 80, 70, 60, 50, 40, 30, 20, 10 or 1 nM as determined by surface plasmon resonance (SPR). The SPR can, for example, be carried out using the protocol described herein.

An aspect of the invention also provides:

A nucleic acid comprising a nucleotide sequence encoding the TCR-Ig, TCR V domain or ligand, optionally comprised by an expression vector for expressing the TCR-Ig, TCR V domain or ligand.

In an example, the nucleic acid is isolated and non-naturally-occurring. In an example, the nucleic acid is DNA, cDNA, RNA or mRNA.

The vector may be a mammalian expression vector, CHO vector or Hek293 vector.

An aspect of the invention also provides:

An engineered immune cell comprising the TCR-Ig, TCR V domain or ligand, or the nucleic acid, wherein the immune cell expresses the TCR-Ig, TCR V domain or ligand, eg, on the cell surface.

In an example, the cell secretes the TCR-Ig, TCR V domain or ligand.

Optionally, the cell is a B-cell, T-cell, NK cell or TIL (tumour infiltrating lymphocyte). The cell may be a human cell, eg, an autologous or allogeneic human cell for use in an ACT method of treating or preventing a cancer or other condition in a human patient.

The invention further provides:

A CAR-T cell comprising a chimaeric antigen receptor (CAR), the receptor comprising an extracellular moiety, a transmembrane moiety and an intracellular signalling moiety, wherein the extracellular moiety comprises the TCR-Ig or TCR V domain, or comprises the ligand first binding site and/or the cell genome comprises the nucleotide sequence of the invention for expressing the TCR-Ig, TCR V domain or ligand first binding site as part of the extracellular moiety of the receptor.

Optionally, the cell is a B-cell, T-cell, NK cell or TIL (tumour infiltrating lymphocyte). The cell may be a human cell, eg, an autologous or allogeneic human cell for use in an ACT method of treating or preventing a cancer or other condition in a human patient.

Thus, for example, the cell is for treating or preventing a disease or condition in a patient, wherein the cell is autologous to the patient, or an allogeneic cell from a donor of the same species as the patient.

Thus, for example, the cell is for administration to a patient to treat or prevent a disease or condition in a human patient, wherein said pMHC antigen comprises an MHC protein sequence that is matched with MHC of the patient.

The invention provides:

A nucleic acid comprising a nucleotide sequence encoding the CAR, eg, an isolated, non-naturally-occurring nucleic acid.

The invention also provides:

A method of identifying an antigen, the method comprising
a. carrying out the method of the invention to select a nucleotide sequence encoding an affinity matured TCR V domain, wherein the antigen is a cell surface-expressed antigen (eg, pMHC comprising a peptide comprising a sequence that is comprised by an antigen expressed by a cancer cell or virally-infected cell);
b. using the selected sequence to produce a second nucleotide sequence encoding a ligand comprising an antigen binding site, wherein the binding site comprises a said affinity matured TCR V domain and binds to the antigen with a binding affinity (KD) of less than 100 nM as determined by surface plasmon resonance (SPR);
c. using the second sequence to express copies of the ligand;
d. providing a plurality of cells comprising cell surface-expressed epitopes (eg, a plurality of cancer cell or virally-infected cells expressing pMHCs);
e. exposing the plurality of cells to copies of said ligand;
f. selecting one or more cells that are specifically bound by ligand; and
g. identifying the cell surface antigen of a said cell that is specifically bound by a ligand; and
h. optionally expressing the antigen or producing nucleic acid comprising nucleotide sequence encoding the antigen (eg, encoding the peptide of a pMHC antigen).

The method is useful for identifying novel targets, eg, novel cell or cancer cell targets that are surface expressed. The invention also, therefore, provides an antigen obtained or obtainable by the method; as well as provides an isolated antibody that specifically binds such an antigen. For example, the antibody is non-naturally occurring. The identification of the cell in step (g) enables isolation of the nucleotide sequence in the cell's genome that encodes the antigen (eg, the peptide of the pMHC). With such knowledge, one can generate the antigen for further immunisation of transgenic non-human animals for antibody generation and/or for phage display (or other display) discovery of antibodies that specifically bind to the novel antigen.

The antigen in step (a) may not be a pre-known antigen. Using the method, it is possible to identify cells that express the antigen naturally. This is useful for finding novel cell-types for targeting medical therapies in humans or animals, or diagnostic methods.

In (b), for example, the binding affinity (KD) is less than 100, 90, 80, 70, 60, 50, 40, 30, 20, 10 or 1 nM as determined by surface plasmon resonance (SPR). The SPR can, for example, be carried out using the protocol described herein.

In (d), for example, the cells are sorted in wells (eg, on a 96 well plate), wherein on average there is no more than one cell type per well.

Optionally the ligand copies can be labelled for identification in the selection of step (f).

Step (f) and/or (g) can be performed using standard methods, eg, using ELISA, SPR or FACS.

In an example, specific binding in (f) is antigen binding of the ligand with a binding affinity (KD) of less than 100 nM as determined by surface plasmon resonance (SPR). For example, the binding affinity (KD) is less than 100, 90, 80, 70, 60, 50, 40, 30, 20, 10 or 1 nM as determined by surface plasmon resonance (SPR). The SPR can, for example, be carried out using the protocol described herein.

In an example, the binding site comprises a TCR Vα domain paired with a TCR Vβ domain, or a TCR Vγ domain paired with a TCR Vδ domain, wherein a vertebrate according to claim 12 is used in the method of step (a).

In an example, the method further comprises using the expressed antigen or nucleic acid to obtain a further antigen-binding ligand (eg, wherein the ligand is or comprises an antibody, antibody VH/VL binding site, antibody V domain, TCR, TCR V/V binding site or TCR V domain), wherein said obtaining comprises immunising a non-human mammal (eg, a mouse or rat that expresses human V domains or a vertebrate according to the invention) with the antigen to produce a plurality of ligands and selecting said further antigen-binding ligand from said plurality, or using the antigen to select the further antigen-binding ligand from an in vitro ligand library (eg, phage display library); and optionally determining the nucleotide sequence(s) encoding the binding site of the further ligand; and optionally expressing copies of the binding site using the determined sequence. A suitable animal is the KYMOUSE™, VELOCIMMUNE MOUSE™, OMNIMOUSE™, OMNIRAT™, OMNIFLIC MOUSE™, MEMO MOUSE™, TRIANNI MOUSE™, HUMAB MOUSE™ or XENOUMOUSE™.

In an example, a locus of the invention comprises a fragment of a human TCRβ locus from TCRBV19 to TCRBJ1-1 inclusive, and optionally up to TCRBJ1-6; eg, up to TCRBJ2-1 or TCRBJ2-7.

In an example, a locus of the invention comprises one or more TCRBJ2 cluster gene segments, eg, TCRBJ2-1 to TCRBJ2-7.

In an example, a locus of the invention comprises (in 5′ to 3′ order) one or more TCRBJ1 cluster gene segments and one or more TCRBJ2 cluster gene segments, wherein there is no TCRBD and/or TCRBC gene segment between the TCRBJ1 cluster gene segment(s) and TCRBJ2 cluster gene segment(s).

In an example, a locus of the invention comprises (in 5′ to 3′ order) one or more TCRBV gene segments, one or more D gene segments and one or more J gene segments. The gene segments have compatible RSS sequences (as will be understood by the skilled addressee); eg, the D segments are antibody DH and/or TCRBD gene segments, and/or the J segments are antibody JH and/or TCRB J gene segments.

Optionally,

(i) each TCRB V is associated with a 23-RSS and each D is associated with a 5′ 12-RSS;
(ii) each TCRB V is associated with a 12-RSS and each D is associated with a 5′ 23-RSS;
(iii) each J is associated with a 23-RSS and each D is associated with a 3′ 12-RSS; or
(iv) each J is associated with a 12-RSS and each D is associated with a 3′ 23-RSS;
optionally wherein each TCRB V is associated with a 23-RSS; each D is associated with a 5′ 12-RSS and a 3′ 23-RSS and each J is associated with a 12-RSS; or each TCRB V is associated with a 23-RSS; each D is associated with a 5′ 12-RSS and a 3′ 12-RSS and each J is associated with a 23-RSS.

In an example, one, more or all of the RSS is a TCR locus RSS. In an alternative example, one, more or all of the RSS is a TCR locus RSS.

In an example, a locus of the invention comprises one or more TCRB promoters in the TCR variable region, eg, a PDβ1 promoter and/or a PDβ2 promoter. For example, the locus comprises a TCR variable region promoter (eg, a PDβ1 promoter or a PDβ2 promoter between the 3′-most TCRV and the 5′-most TCR D, when the variable region is a TCRB region). Alternatively, the promoter is an antibody heavy chain variable region promoter, eg, PDQ52.

Optionally said D gene segments are TCR gene segments, eg, TCRBD gene segments and said J gene segments are TCR J segments, eg, TCRBJ gene segments.

In an example, a locus of the invention comprises (in 5′ to 3′ order) one or more TCRBV gene segments, one or more TCRBD gene segments and one or more TCRBJ gene segments.

In an example, a locus of the invention comprises (in 5′ to 3′ order) a TCRBD1 gene segment and a TCRBD2 gene segment wherein there is no TCRBJ and/or TCRBC gene segment between the TCRBD1 and TCRBD2 gene segments.

In an example, a locus of the invention comprises one or more trypsinogen exons (eg, T4-T8; or T4, T6 and T8), eg, between a TCRBV gene segment and TCRBD gene segment of the locus.

In an example, a locus of the invention comprises (eg, in 5′ to 3′ order) TCRBV 19, 20-1, 24-1, 25-1, 27, 28 and 29-1 (and optionally 30). In an embodiment, the locus also comprises one more or all of TCRBV 21-1, 22-1 and 26. Optionally in this example the C region is an antibody IgH C region.

In an example, a locus of the invention comprises (eg, in 5′ to 3′ order) TCRBV 5-4, 6-6, 5-5, 7-6, 5-6, 6-8, 7-7, 6-9, 7-8, 5-8, 7-9, 13, 10-3, 11-3, 12-3, 12-4, 12-5, 14, 15, 18, 19, 20-1, 24-1, 25-1, 27, 28 and 29-1 (or this list but omitting up to 5 of the listed V gene segments). Optionally in this example the C region is an antibody IgH C region.

In an example, a locus of the invention comprises (eg, in 5′ to 3′ order) TCRBV 2, 3-1, 4-1, 5-1, 6-1, 4-2, 4-3, 6-3, 7-2, 6-4, 7-3, 9, 11-1, 10-2, 11-2, 6-5, 5-4, 6-6, 5-5, 7-6, 5-6, 6-8, 7-7, 6-9, 7-8, 5-8, 7-9, 13, 10-3, 11-3, 12-3, 12-4, 12-5, 14, 15, 18, 19, 20-1, 24-1, 25-1, 27, 28 and 29-1 (or this list but omitting up to 10 or 5 of the listed V gene segments. Optionally in this example the C region is an antibody IgH C region.

In an example, a locus of the invention comprises a fragment of a human TCRα locus from TCRAV24 to TCRAJ61 inclusive, and optionally up to TCRAJ50, 40, 30, 20, 20 or 1. Optionally in this example the C region is an antibody IgL C region.

In an example, a locus of the invention comprises no TCRDV, D, J and/or C gene segment between the 3′-most TCRAV and the 5′-most TCRAJ. For example, there is no TCRDV or C gene segment between the 3′-most TCRAV and the 5′-most TCRAJ. Optionally in these examples the C region is an antibody IgH C region.

In an example, a locus of the invention comprises (in 5′ to 3′ order) one or more TCRAV gene segments and one or more J gene segments

### Wherein

(i) each TCRA V is associated with a 23-RSS and each J is associated with a 5′ 12-RSS; or
(ii) each TCRA V is associated with a 12-RSS and each J is associated with a 5′ 23-RSS.

Optionally in this example the C region is an antibody IgL C region. In an example, one, more or all of the RSS is a TCR locus RSS.

In an example, a locus of the invention comprises one or more TCRA promoters in the TCR variable region, eg, a TEA promoter and/or a PJα49 promoter. For example, the locus comprises a TCR variable region promoter (eg, TEA promoter or a PJα49 promoter between the 3′-most TCRV and the 5′-most TCR J, when the variable region is a TCRBA region). Alternatively, the promoter is an antibody kappa variable region promoter.

Optionally, said J gene segments are TCRJ gene segments, eg, TCRAJ gene segments.

In an example, a locus of the invention comprises (eg, in 5′ to 3′ order) TCRAV 23, 24, 25, 26-1, 27, 30, 26-2, 34, 35, 36, 38-1, 38-2, 39, 40 and 41 (and optionally one or more of 28, 29, 31-33 and 37)—or this list but omitting up to 5 of the listed V gene segments.

In an example, a locus of the invention comprises (eg, in 5′ to 3′ order) TCRAV 1-1, 1-2, 2, 3, 4, 5, 6, 7, 8-1, 9-1, 10, 11, 12-1, 8-2, 8-3, 13-1, 12-2, 8-4, 13-2, 14, 9-2, 12-3, 8-6, 16, 17, 19, 19, 20, 21, 22, 23, 24, 25, 26-1, 27, 30, 26-2, 34, 35, 36, 38-1, 38-2, 39, 40 and 41 (and optionally one or more of 28, 29, 31-33 and 37)—or this list but omitting up to 10 or 5 of the listed V gene segments.

In any locus herein, optionally the locus does not comprise a TCR C gene segment downstream of the 3-most TCR J gene segment. This can express, for example, a TCRV-Ig.

In an example, a locus of the invention comprises a TCR promoter, eg, a T early alpha (TEA) promoter eg, when TCRA V region gene segments are comprised by the V region of the locus. Optionally, the promoter is between the 3′-most TCRV and the 5′-most D or J gene segment.

The invention provides a non-human vertebrate or non-human vertebrate cell (eg, a B-cell or CHO cell) that comprises a rearranged TCR V region that is expressible to produce one or more in-frame transcripts comprising a TCR V region nucleotide sequence spliced to a nucleotide sequence encoding an Ig constant region (eg, a Cμ region or Cγ region).

In an embodiment of the method or vertebrate herein, said vertebrate expresses a plurality of different rearranged TCR VDJ, wherein each VDJ is the product of rearrangement of a V, D and J, wherein the V/J are selected from the group consisting of TCRBV27/TCRBJ1-5, TCRBV27/TCRBJ1-1, TCRBV20-1/TCRBJ1-5, TCRBV20-1/TCRBJ1-2, TCRBV20-1/TCRBJ1-4, TCRBV29-1/TCRBJ1-5, TCRBV28/TCRBJ1-5, TCRBV20-1/TCRBJ1-1, TCRBV27/TCRBJ1-2 and TCRBV29-1/TCRBJ1-4. This is demonstrated in Example 3, wherein such a vertebrate advantageously provides one or more of the following features:—
human TCR exons that are able to rearrange at an ectopic (ie, non-naturally-occurring) genomic position;
rearranged human TCR exons are able to express from an ectopic genomic position;
rearranged TCR V regions are used to and produce in-frame transcripts, spliced to a nucleotide sequence encoding an Ig constant region (for example a Cμ region);
endogenous non-human vertebrate Ig regulatory elements are able to drive expression of transcripts comprising TCR variable regions;
dominant usage of some exons and lower usage of others that usefully reflects dominance of certain V and J exons occur at the equivalent native human TCR locus;
the human TCR variable region is advantageously acted on by orthologous, non-human vertebrate factors;
rearranged TCR V region equences comprise nucleotide addition and/or deletions at V-to-D and D-to-J junctions, thus generating CDR3 diversity as occurs during VDJ rearrangement at the native non-human vertebrate IgH locus; and
human TCR-like CDR3 length data (which indicates potential for TCR-like binding preferences).

The invention provides a plurality of B-cells (eg, rodent, mouse or rat B-cells) comprising one or more immunoglobulin loci that comprise recombined TCR variable regions, wherein the variable regions comprise TCR gene segment junctional mutation. The invention also provides a non-human vertebrate that comprises a plurality of B-cells, the B-cells comprising one or more immunoglobulin loci that comprise recombined TCR variable regions, wherein the variable regions comprise TCR gene segment junctional mutation. For example, the B-cells comprise a said immunoglobulin locus in homozygous state, eg, a homozygous IgH or IgL locus. For example, the B-cells comprise a said immunoglobulin locus in heterozygous state, eg, a heterozygous IgH or IgL locus; for example, one allele of the IgH locus comprises antibody VDJ gene segments and the other allele comprises TCR VDJ or VJ gene segments (eg, TCRB VDJ gene segments).

In an example of the invention, the B-cells express a plurality of mRNA transcripts (eg, mu or gamma transcripts), each mRNA transcript being expressed from a respective B-cell and comprising a TCR V region nucleotide sequence (eg, a TCRB V region sequence), wherein the V region nucleotide sequence is a transcript of a recombined variable region comprised by the genome of said respective cell, wherein the recombined variable region is a product of the recombination of (i) a TCR (eg, TCRB or TCRD) V gene segment, a TCR D gene segment and a TCR J gene segment, wherein the recombined variable region comprises gene segment junctional mutation; or a TCR (eg, TCRA or TCRC) V gene segment and a TCR J gene segment, wherein the recombined variable region comprises gene segment junctional mutation.

In an embodiment (as demonstrated in Example 3), the junctional mutation of said recombined variable region comprises V segment nucleotide deletion. Additionally or alternatively, the junctional mutation of said recombined variable region comprises V segment nucleotide addition. Additionally or alternatively the junctional mutation of said recombined variable region comprises D segment nucleotide deletion. Additionally or alternatively the junctional mutation of said recombined variable region comprises D segment nucleotide addition. Additionally or alternatively the junctional mutation of said recombined variable region comprises J segment nucleotide deletion. Additionally or alternatively the junctional mutation of said recombined variable region comprises J segment nucleotide addition. Additionally or alternatively the junctional mutation of said recombined variable region comprises V-D junction nucleotide deletion. Additionally or alternatively the junctional mutation of said recombined variable region comprises V-D junction nucleotide addition. Additionally or alternatively the junctional mutation of said recombined variable region comprises D-J junction nucleotide deletion. Additionally or alternatively the junctional mutation of said recombined variable region comprises D-J junction nucleotide addition. In an example, the junctional mutation comprises 5′ and/or 3′ D nucleotide addition; 5′ nucleotide addition and 3′ nucleotide deletion; or 5′ nucleotide deletion and 3′ nucleotide addition.

Optionally (as demonstrated in Example 3), the plurality of mRNA transcripts comprise a plurality of different said TCR V region nucleotide sequences, wherein a subset of said plurality of TCR V region nucleotide sequences are transcripts of rearranged variable regions comprising TCR V segment nucleotide deletions, wherein said subset comprises (i) members each comprising 1 such deletion (ie, a deletion of one such nucleotide), (ii) members each comprising 2 such deletions (ie, a deletion of 2 such nucleotides), (iii) members each comprising 3 such deletions, (iv) members each comprising 4 such deletions, (v) members each comprising 5 such deletions, (vi) members each comprising 6 such deletions, (vii) members each comprising 7 such deletions, (viii) members each comprising 8 such deletions and optionally (ix) members each comprising 9 such deletions. The term “comprising” in this context, means that there is no more or less than the recited number of mutations (eg, deletions). Hence, “members each comprising 2 such deletions” means that each member has 2 (and not more or less) V deletions compared to the corresponding germline V gene segment sequence.

Optionally (as demonstrated in Example 3),

the members of (i) are comprised by said plurality of mRNA transcripts at first percentage;
the members of (ii) are comprised by said plurality of mRNA transcripts at second percentage;
the members of (iii) are comprised by said plurality of mRNA transcripts at third percentage;
the members of (iv) are comprised by said plurality of mRNA transcripts at fourth percentage;
the members of (v) are comprised by said plurality of mRNA transcripts at fifth percentage;
the members of (vi) are comprised by said plurality of mRNA transcripts at sixth percentage;
the members of (vii) are comprised by said plurality of mRNA transcripts at seventh percentage;
the members of (viii) are comprised by said plurality of mRNA transcripts at eighth percentage;
the members of (ix) are comprised by said plurality of mRNA transcripts at ninth percentage; and
the average of the first to ninth percentages of is from 5-10% (eg, 10%) and/or the average of the second to sixth percentages is from 10-15% (eg, 14%). The term “the average of the second to sixth percentages” means the average of the second, third, fourth, fifth and sixth percentages (ie, calculated by adding these percentages together and dividing by the number of percentage values, in this case dividing by 5).

Optionally (as demonstrated in Example 3), the plurality of mRNA transcripts comprise a plurality of different said TCR V region nucleotide sequences, wherein a subset of said plurality of TCR V region nucleotide sequences are transcripts of rearranged variable regions comprising TCR D segment nucleotide deletions at the 5′ end of the D segment, wherein said subset comprises (a) members each comprising 1 such deletion, (b) members each comprising 2 such deletions, (c) members each comprising 3 such deletions, (d) members each comprising 4 such deletions, (e) members each comprising 5 such deletions, (f) members each comprising 6 such deletions, (g) members each comprising 7 such deletions and optionally (h) members each comprising 8 such deletions.

The cells or vertebrate of claim 94, wherein

the members of (a) are comprised by said plurality of mRNA transcripts at first percentage;
the members of (b) are comprised by said plurality of mRNA transcripts at second percentage;
the members of (c) are comprised by said plurality of mRNA transcripts at third percentage;
the members of (d) are comprised by said plurality of mRNA transcripts at fourth percentage;
the members of (e) are comprised by said plurality of mRNA transcripts at fifth percentage;
the members of (f) are comprised by said plurality of mRNA transcripts at sixth percentage;
the members of (g) are comprised by said plurality of mRNA transcripts at seventh percentage;
the members of (h) are comprised by said plurality of mRNA transcripts at eighth percentage; and
the average of the first to eighth percentages of is from 5-10% (eg, 7%) and/or the fourth percentage is from 10-17% (eg, 16%).

Optionally (as demonstrated in Example 3), the plurality of mRNA transcripts comprise a plurality of different said TCR V region nucleotide sequences, wherein a subset of said plurality of TCR V region nucleotide sequences are transcripts of rearranged variable regions comprising TCR D segment nucleotide deletions at the 3′ end of the D segment, wherein said subset comprises (a′) members each comprising 1 such deletion, (b′) members each comprising 2 such deletions, (c′) members each comprising 3 such deletions, (d′) members each comprising 4 such deletions, (e′) members each comprising 5 such deletions, (f′) members each comprising 6 such deletions, (g′) members each comprising 7 such deletions and optionally (h′) members each comprising 8 such deletions.

The cells or vertebrate of claim 96, wherein

the members of (a′) are comprised by said plurality of mRNA transcripts at first percentage;
the members of (b′) are comprised by said plurality of mRNA transcripts at second percentage;
the members of (c′) are comprised by said plurality of mRNA transcripts at third percentage;
the members of (d′) are comprised by said plurality of mRNA transcripts at fourth percentage;
the members of (e′) are comprised by said plurality of mRNA transcripts at fifth percentage;
the members of (f′) are comprised by said plurality of mRNA transcripts at sixth percentage;
the members of (g′) are comprised by said plurality of mRNA transcripts at seventh percentage;
the members of (h′) are comprised by said plurality of mRNA transcripts at eighth percentage; and
the average of the first to eighth percentages of is from 5-10% (eg, 9%) and/or the average of the second and third percentages is 15-20% (eg, 16%).

Optionally (as demonstrated in Example 3), the plurality of mRNA transcripts comprise a plurality of different said TCR V region nucleotide sequences, wherein a subset of said plurality of TCR V region nucleotide sequences are transcripts of rearranged variable regions comprising TCR J segment (eg, TCRBJ) nucleotide deletions, wherein said subset comprises (aa) members each comprising 1 such deletion, (bb) members each comprising 2 such deletions, (cc) members each comprising 3 such deletions, (dd) members each comprising 4 such deletions, (ee) members each comprising 5 such deletions, (ff) members each comprising 6 such deletions, (gg) members each comprising 7 such deletions and optionally (hh) members each comprising 8 such deletions.

The cells or vertebrate of claim 98, wherein

the members of (aa) are comprised by said plurality of mRNA transcripts at first percentage;
the members of (bb) are comprised by said plurality of mRNA transcripts at second percentage;
the members of (cc) are comprised by said plurality of mRNA transcripts at third percentage;
the members of (dd) are comprised by said plurality of mRNA transcripts at fourth percentage;
the members of (ee) are comprised by said plurality of mRNA transcripts at fifth percentage;
the members of (ff) are comprised by said plurality of mRNA transcripts at sixth percentage;
the members of (gg) are comprised by said plurality of mRNA transcripts at seventh percentage;
the members of (hh) are comprised by said plurality of mRNA transcripts at eighth percentage; and
the average of the first to eighth percentages of is from 5-10% (eg, 9) and/or the average of the fourth to sixth percentages is 10-15% (eg, 13%).

Optionally (as demonstrated in Example 3), the plurality of mRNA transcripts comprise a plurality of different said TCR V region nucleotide sequences, wherein a subset of said plurality of TCR V region nucleotide sequences are transcripts of rearranged variable regions comprising TCR V-D junction insertions, wherein said subset comprises (A) members each comprising 1 such insertion, (B) members each comprising 2 such insertions, (C) members each comprising 3 such insertions, (D) members each comprising 4 such insertions, (E) members each comprising 5 such insertions, (F) members each comprising 6 such insertions, (G) members each comprising 7 such insertions, (H) members each comprising 8 such insertions, (I) members each comprising 9 such insertions, (J) members each comprising 10 such insertions and optionally (K) members each comprising 11 such insertions.

The cells or vertebrate of claim 100, wherein

the members of (A) are comprised by said plurality of mRNA transcripts at first percentage;
the members of (B) are comprised by said plurality of mRNA transcripts at second percentage;
the members of (C) are comprised by said plurality of mRNA transcripts at third percentage;
the members of (D) are comprised by said plurality of mRNA transcripts at fourth percentage;
the members of (E) are comprised by said plurality of mRNA transcripts at fifth percentage;
the members of (F) are comprised by said plurality of mRNA transcripts at sixth percentage;
the members of (G) are comprised by said plurality of mRNA transcripts at seventh percentage;
the members of (H) are comprised by said plurality of mRNA transcripts at eighth percentage;
the members of (I) are comprised by said plurality of mRNA transcripts at ninth percentage;
the members of (J) are comprised by said plurality of mRNA transcripts at tenth percentage;
the members of (K) are comprised by said plurality of mRNA transcripts at eleventh percentage; and
the average of the first to eleventh percentages of is from 5-10% (eg, 7%) and/or the average of the second to fourth percentages is 12-17% (eg, 15%).

Optionally (as demonstrated in Example 3), the plurality of mRNA transcripts comprise a plurality of different said TCR V region nucleotide sequences, wherein a subset of said plurality of TCR V region nucleotide sequences are transcripts of rearranged variable regions comprising TCR D-J junction insertions, wherein said subset comprises (A′) members each comprising 1 such insertion, (B′) members each comprising 2 such insertions, (C′) members each comprising 3 such insertions, (D′) members each comprising 4 such insertions, (E′) members each comprising 5 such insertions, (F′) members each comprising 6 such insertions, (G′) members each comprising 7 such insertions, (H′) members each comprising 8 such insertions, (I′) members each comprising 9 such insertions, (J′) members each comprising 10 such insertions and optionally (K′) members each comprising 11 such insertions.

The cells or vertebrate of claim 102, wherein

the members of (A′) are comprised by said plurality of mRNA transcripts at first percentage;
the members of (B′) are comprised by said plurality of mRNA transcripts at second percentage;
the members of (C′) are comprised by said plurality of mRNA transcripts at third percentage;
the members of (D′) are comprised by said plurality of mRNA transcripts at fourth percentage;
the members of (E′) are comprised by said plurality of mRNA transcripts at fifth percentage;
the members of (F′) are comprised by said plurality of mRNA transcripts at sixth percentage;
the members of (G′) are comprised by said plurality of mRNA transcripts at seventh percentage;
the members of (H′) are comprised by said plurality of mRNA transcripts at eighth percentage;
the members of (I′) are comprised by said plurality of mRNA transcripts at ninth percentage;
the members of (J′) are comprised by said plurality of mRNA transcripts at tenth percentage;
the members of (K′) are comprised by said plurality of mRNA transcripts at eleventh percentage; and
the average of the first to eleventh percentages of is from 5-10% (eg, 7%) and/or the average of the first to fourth percentages is 10-15% (eg, 11%).

Optionally (as demonstrated in Example 3), the plurality of mRNA transcripts comprise a plurality of different said TCR V region nucleotide sequences, wherein a subset of said plurality of TCR V region nucleotide sequences encode TCRB V domains each comprising a CDR3 having a length in the range of from 8 to 16 amino acids.

Optionally (as demonstrated in Example 3), the plurality of mRNA transcripts comprise a plurality of different said TCR V region nucleotide sequences, wherein said plurality of TCR V region nucleotide sequences encode TCRB V domains each comprising a CDR3, wherein the V domains most commonly comprise a CDR3 length of 11, 12 or 13 amino acids. In an example, the TCRB V domains have a most common CDR3 length of 12 amino acids (see, eg, Example 3 where this is exemplified).

In an example, the vertebrate expresses, or the B-cells express, TCRB V domains and the TCRB V domains most commonly comprise a CDR3 length of 11, 12 or 13 amino acids. In an embodiment, the invention provides a non-human vertebrate or a non-human vertebrate cell (eg, a mouse or a mouse cell) that comprises a rearranged TCRB variable region that is ectopically positioned in the genome of the vertebrate of cell, wherein the vertebrate or cell expresses TCRB V domains comprising most commonly a CDR3 length of 11, 12 or 13 amino acids, eg, of 12 amino acids.

Optionally, said plurality of mRNA transcripts are transcripts of CD19+ B-cells obtainable by FACs sorting.

In an example, said B-cells herein comprise or consist of bone marrow B-cells.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published byJones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It will be understood that particular configurations, aspects, examples, embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non limiting Examples.

### EXAMPLES

### Experimental Techniques

In some embodiments of the invention, the non-human host animal will be a mouse and the non-host DNA that is introduced will be human. The majority of experimental techniques that will be utilised for the creation of TCR-Ig transgenic mice are as described as described in Lee et al, Nat Biotechnol. 2014 April; 32(4):356-63. doi: 10.1038/nbt.2825. Epub 2014 Mar. 16, “Complete humanization of the mouse immunoglobulin loci enables efficient therapeutic antibody discovery” and in WO2011/004192. For example:
BAC ordering and isolation of human BAC DNA
Embryonic Stem cell (ES cell) lines used
Recombineering protocols and cassettes
The use of an initiation cassette (termed “landing pad”) for insertion of the first DNA fragment into an endogenous mouse antibody locus
The use of the SSR-based technique termed sequential recombinase-mediated cassette exchange (sRMCE) to allow continuous, sequential insertion of BAC inserts into the same locus
The optional targeting of a “flip-over” cassette into a 5′ region away from the endogenous VDJ or VJ sequences, such that these regions may be inactivated via a Lox-CRE mediated inversion.

Methods below refer to targeting human TCR beta gene segments into an IgH locus, and human TCR alpha gene segments into an Ig kappa locus. However, the reverse configuration may be carried out instead. Suitable mouse ES cells are AB2.1 cells or F1H4 cells. If desired, BAC DNA insertions could be performed using Cas9 target genome cleavage.

### Example 1: Mouse IgH Locus Modification

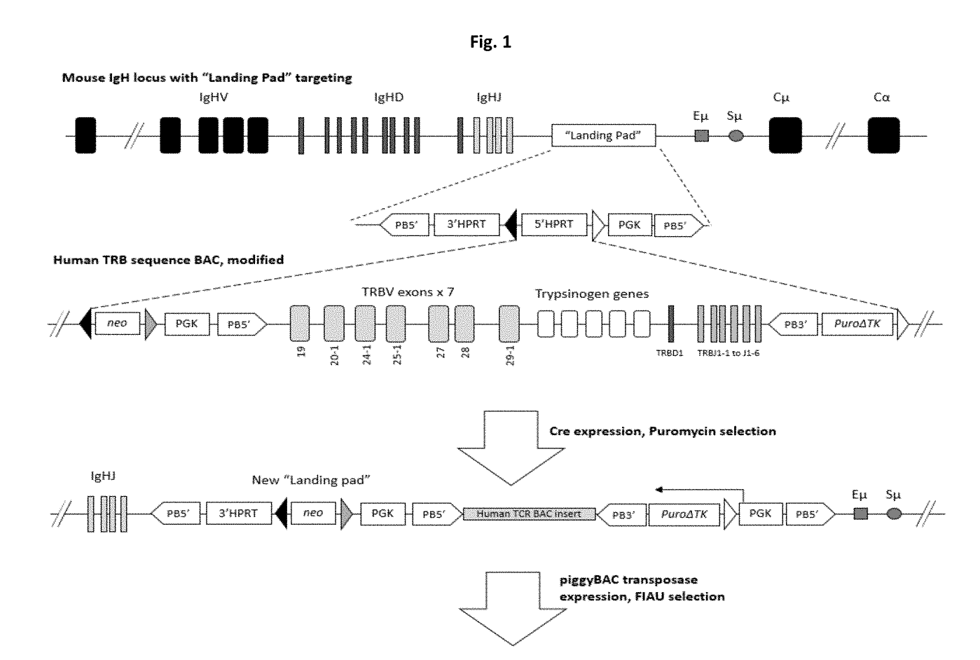

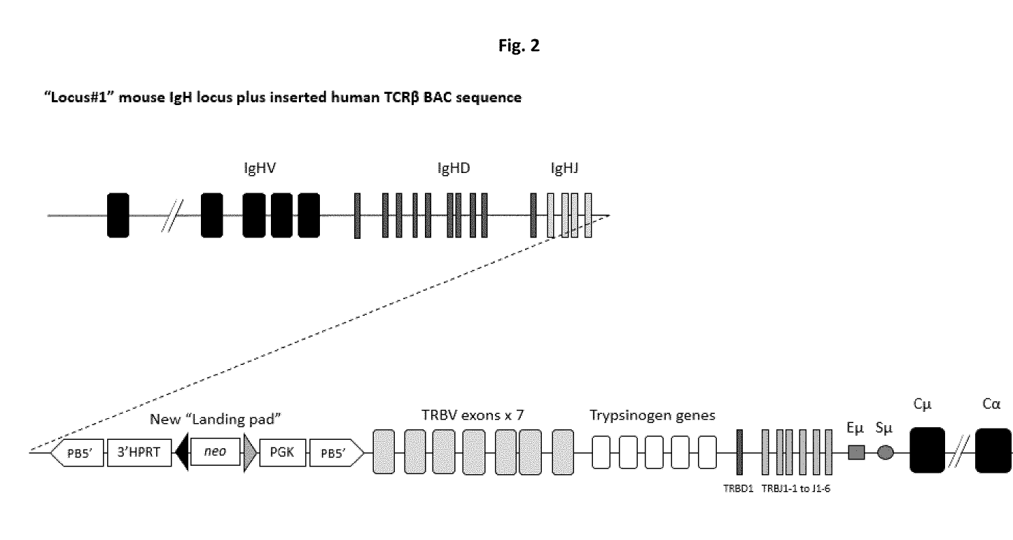

FIG. 1 shows a summary of the sRMCE targeting strategy and ES cell selection protocol that will be used, for modification of the mouse IgH locus via introduction of DNA sequence from the human T-cell Receptor Beta (TRB) locus.

In this embodiment, the BAC used is the human BAC clone CH17-318M05, obtained from the BACPAC Resources Centre at the Children's Hospital Oakland Research Institute (CHORI), Oakland, Calif. This BAC clone contains a large region of the human TRB locus.

Standard Recombineering techniques were used to modify BAC CH17-318M05 via the introduction of 5′ and 3′ modifications as used in WO2011/004192.

As shown in FIG. 1, the 5′ modification was introduced upstream of the TRBV19 exon and the 3′ modification was introduced downstream of the first TRBJ gene cluster, i.e. downstream of the TRBJ1-6 gene but upstream of the TRBC1 Constant region gene.

The BAC DNA region flanked by these 5′ and 3′ modifications therefore comprises seven TRBV genes, a single TRBD gene and six TRBJ genes. The order of TRB genes within this flanked region essentially matches that of the V, D and J genes of the endogenous mouse IgH genomic locus, i.e. the ordering of genes is V-D-J in a 5′ to 3′ direction. There are no intervening TRB genes within this flanked region which disrupt this order. By comparison, the full human TRB locus contains a second Diversity gene TRBD2, and a second TRBJ gene cluster; separating these genes from the TRBD1 and TRBJ1 genes is the TRBC1 Constant region gene.

The flanked BAC DNA sequence also includes a region containing Trypsinogen genes including PRSS1 (Trypsinogen 1) and PRSS2 (Trypsinogen 2). In present Example, this region has been retained, in other embodiments of the invention this region may be modified or deleted.

As shown in FIG. 1, the modified human BAC clone was targeted into a “landing pad” sequence inserted within the endogenous mouse IgH locus, using sRMCE methods described in WO2011/004192. Following Lox-Cre mediated BAC DNA insertion and puromycin selection, the nature of the BAC DNA insert plus 5′ and 3′ flanking modifications is depicted.

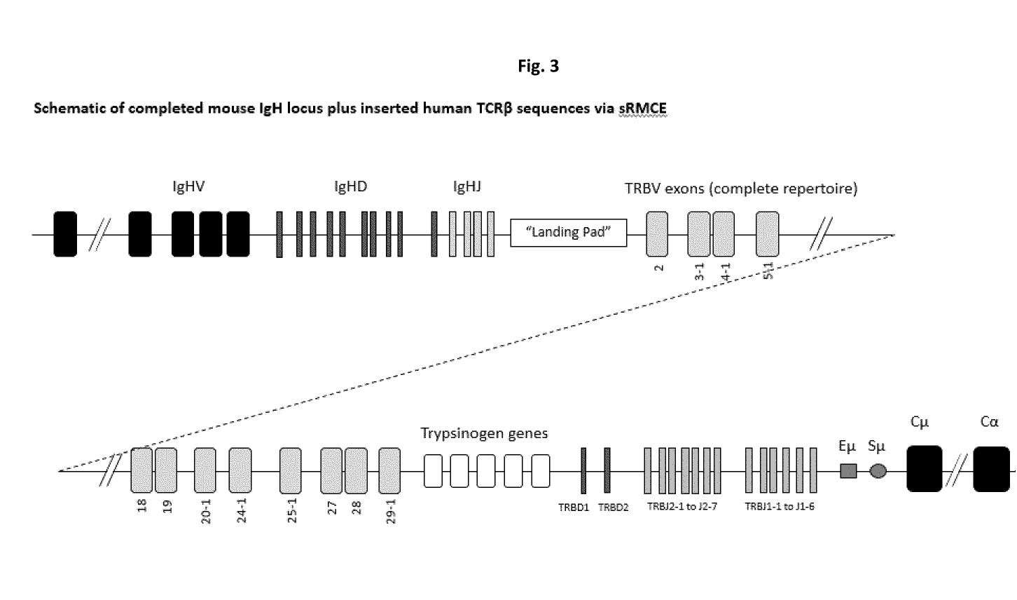

FIG. 2 shows the appearance of the BAC DNA insert following PiggyBac transposase-mediated excision of the 3′ flanking modification, which removes the 3′ modification. The resulting chimeric locus contains seven human TRBV genes, a single human TRBD gene and six human TRBJ genes, inserted 5′ of the endogenous mouse IgH Eμ enhancer. This chimeric locus (termed the Locus#1) is to be immediately analysed in mice for the occurrence of human TRB BAC insert VDJ recombination events in mouse B-cells. The 5′ and 3′ ends of the region of human TRB BAC sequence incorporated into the mouse IgH locus are defined by the following sequences:

5′ end:

(SEQ ID NO: 1)

Taatcagagtatctacagcgctcctggagtaacccaaaccttatggttgc
tggaaagataaaaaaccaat
3′ end:

(SEQ ID NO: 2)

Cagcttgttgctctgctgtgttgcctgcagttcctcagctgtagagctcc
ttgcttagtcttcagggctg

In this Example, the endogenous mouse V, D and J genes at this chimeric locus have not been modified. In other embodiments of the invention, the endogenous mouse V, D and J genes at this chimeric locus may be deleted either whole or in part, or inactivated through inversion or another modification.

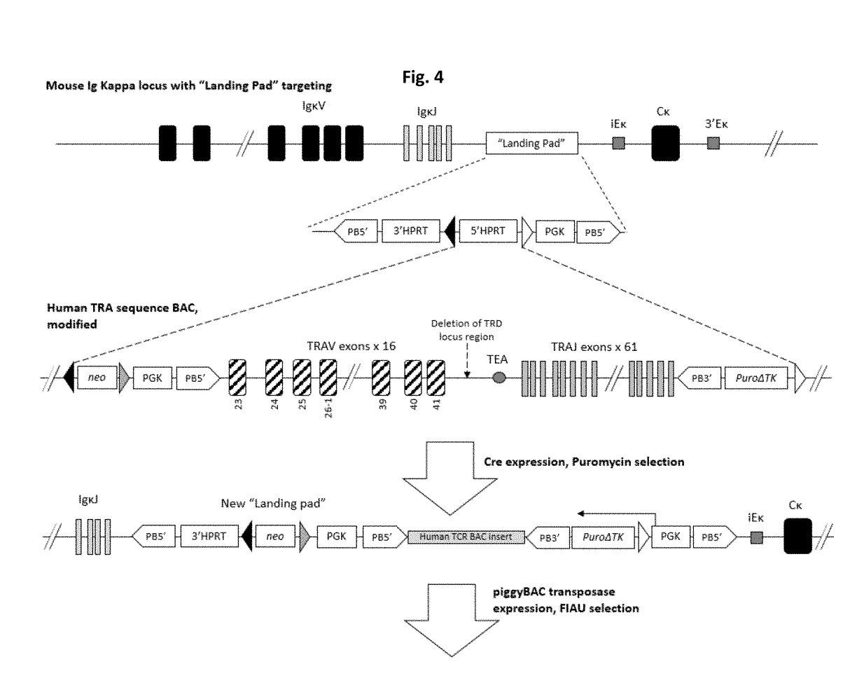

FIG. 3 depicts an embodiment of the invention in which the endogenous mouse IgH locus will be modified to include a complete repertoire of functional human TRB variable region segments. In this embodiment, the organisation of the TRBD and TRBJ genes will be altered so as to resemble the ordering of endogenous IgH V, D and J genes: that is, TRBD1 and TRBD2 are located together as a continuous cluster, 3′ of which are the thirteen TRBJ genes also placed together as a continuous cluster. The TRBC Constant region genes will have been removed.

The DNA modifications required to achieve the above configuration may be achieved via multiple methods. In one method, BAC DNA incorporating the TRBD2 and TRBJ2 gene segments may be targeted into the chimeric locus depicted in FIG. 2. In another method, the human BAC CH17-318M05 may be modified by recombineering to achieve the desired configuration, prior to targeting into ES cells.

In another embodiment, the configuration of the D and J genes may be changed such that the configuration from 5′ to 3′ is D1, D2, J1-1 to J1-6, J2-1 to J2-7.

Subsequent BAC inserts containing the full repertoire of human TRBV genes may be added via SRMCE methods as described in WO2011/004192.

### Example 2: Mouse Ig Kappa Locus Modification

### A: Insertion of Unrearranged TCR V Region Gene Segments

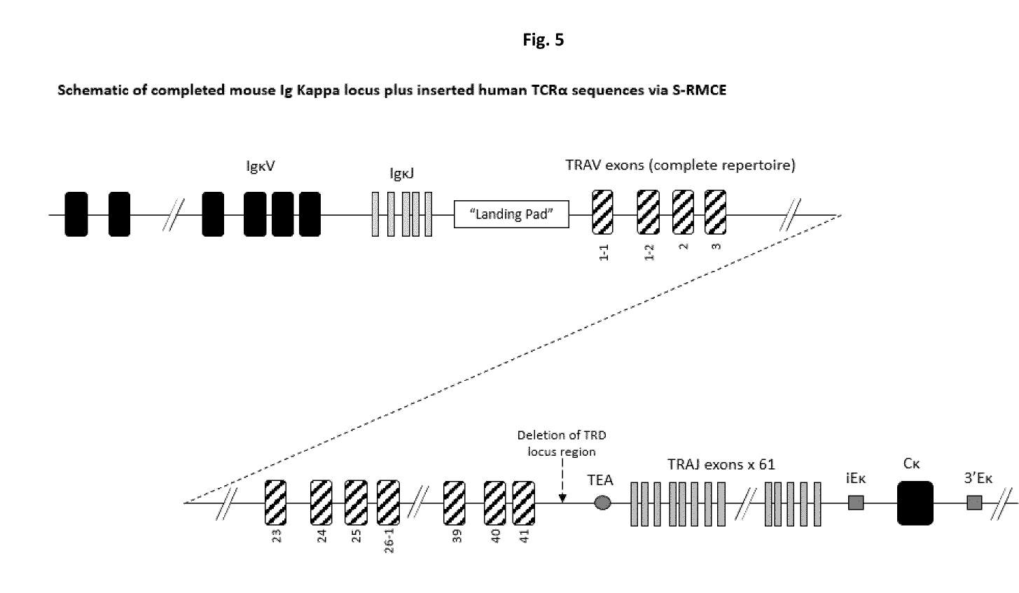

FIG. 4 shows a summary of the sRMCE targeting strategy and ES cell selection protocol that will be used in one embodiment of the invention, for modification of the mouse Igκ locus via introduction of DNA sequence from the human T-cell Receptor Alpha/Delta (TRA/D) locus.

In one embodiment, two BAC clones will be used, obtained from the BACPAC Resources Centre at the Children's Hospital Oakland Research Institute (CHORI), Oakland, Calif. The BAC clones that will be used are CH17-294L13 and CH17-272613. Each BAC clone contains a large region of the human TRA/D locus.

In this Example, recombineering and/or other standard molecular cloning methods known to those skilled in the art shall be used to insert approximately 76 kilobases of DNA from BAC clone CH17-272613 into BAC clone CH17-294L13. This insert will include the “T early alpha” (TEA) promoter plus the full repertoire of 61 functional TRAJ genes. The insert shall be placed downstream of the T-cell Receptor Alpha (TRA) V41 gene.

In addition, recombineering techniques will used to modify the sequence of the above fusion BAC via the introduction of 5′ and 3′ modifications as used in WO2011/004192.

As shown in FIG. 4, the 5′ modification will be introduced upstream of the TRAV23 exon and the 3′ modification will be introduced downstream of the TRAJ gene cluster.

The BAC DNA region flanked by these 5′ and 3′ modifications comprises approximately seventeen functional TRAV genes, and TRAJ genes. The order of TRA genes within this flanked region essentially matches that of the V and J genes of the endogenous mouse Igκ genomic locus, i.e. the ordering of genes is V-J in a 5′ to 3′ direction. As shown in FIG. 4, the fusion BAC sequence differs from the endogenous human TRA/D locus sequence in that a large region of DNA including almost the entirety of the TCR Delta genes is absent.

As shown in FIG. 4, the modified human BAC clone will be targeted into a “landing pad” sequence inserted within the endogenous mouse Igκ locus, using sRMCE methods described in WO2011/004192. Following Lox-Cre mediated BAC DNA insertion and puromycin selection, the nature of the BAC DNA insert plus 5′ and 3′ flanking modifications is depicted.

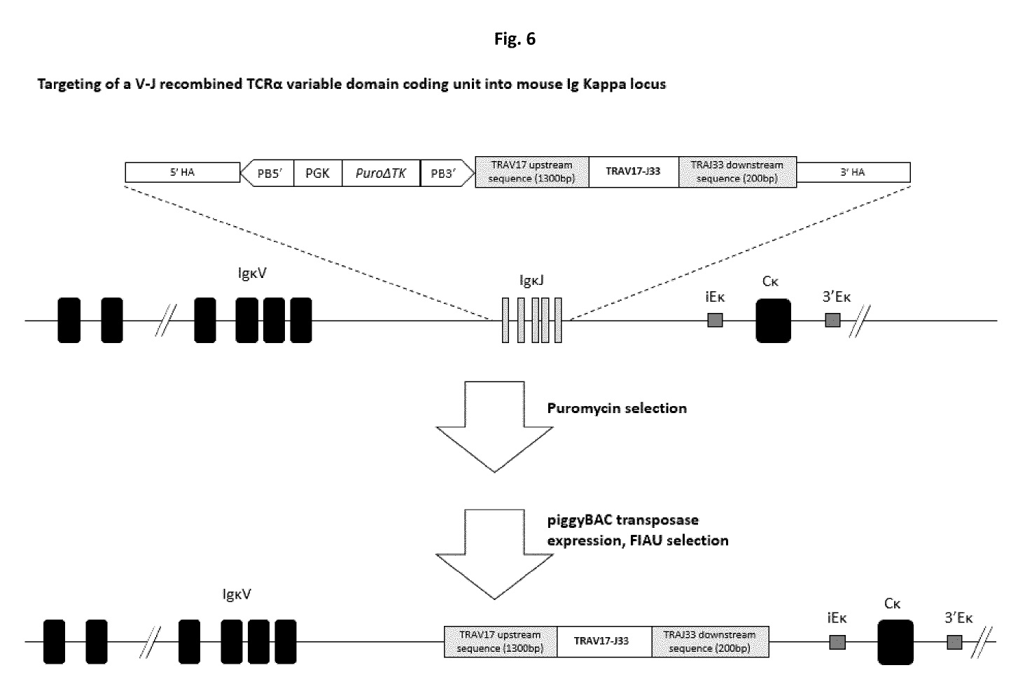

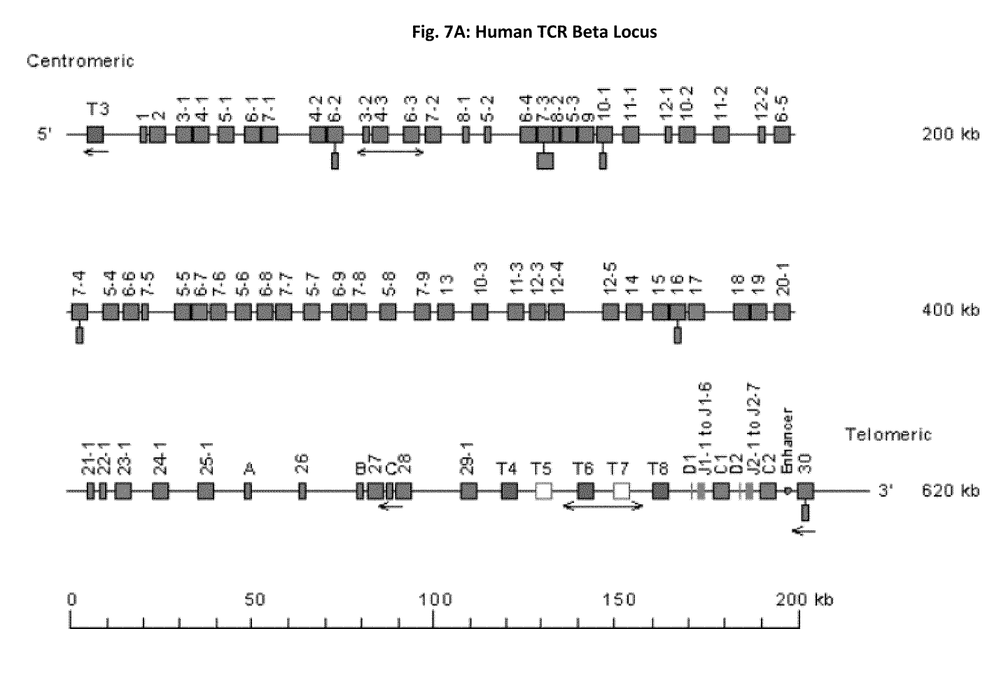

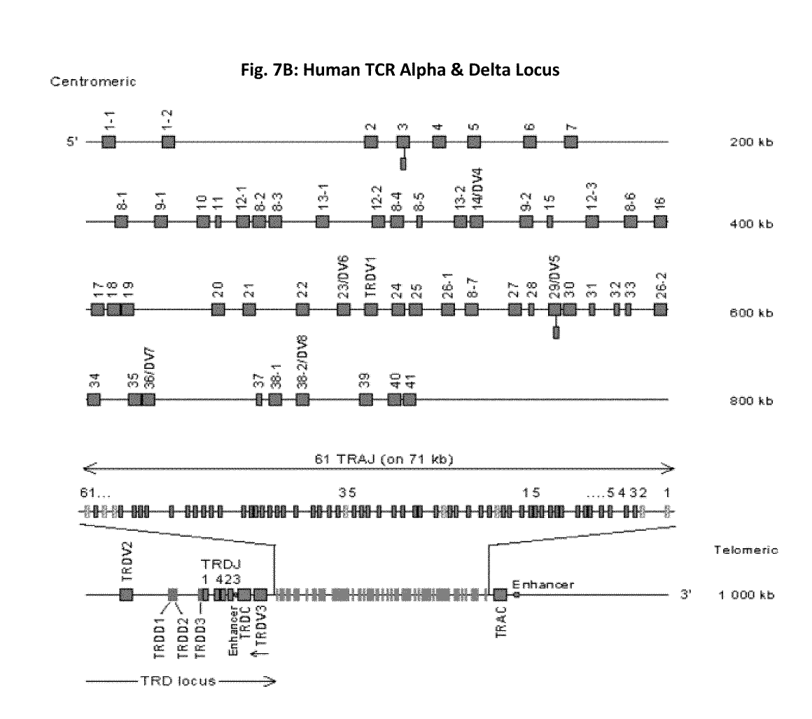

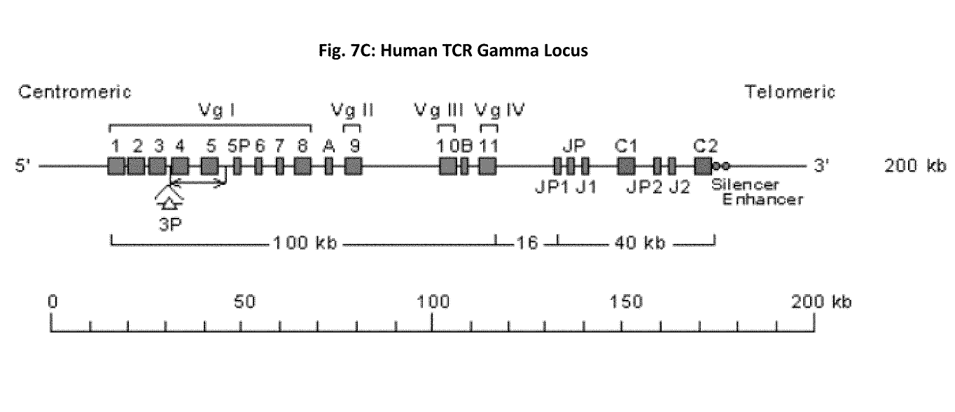

FIG. 5 depicts an embodiment of the invention in which the endogenous mouse Igκ locus will be modified to include a complete repertoire of functional human TRA variable region segments. Subsequent BACs containing the full repertoire of human TRAV genes may be added via SRMCE methods as described in WO2011/004192.

One embodiment of a completed TCR-Ig transgenic non-human animal will be a mouse which is homozygous for both the chimeric IgH locus and a chimeric Igκ locus. It is expected that such an animal shall produce TCR-Ig antibodies incorporating human TCR variable domains.

In embodiments, the Ig lambda locus has been deleted or otherwise inactivated, in order to force gene expression from the chimeric kappa locus. In embodiments, the Ig lambda locus is modified to produce a TCR-Ig chimeric locus via introduction of human TCR alpha or delta V and J genes in operable connection with an antibody Cλ.

### B: Insertion of a Rearranged TCR VJ

Reference is made to FIG. 6. The DNA sequence used for the TRAV17-J33 variable domain coding unit (VDCU) was designed with the inclusion of a CDR3 sequence reported in Brennan R M et al, J Virol. 2007 July; 81(13):7269-73. Epub 2007 Apr. 25, “Predictable alphabeta T-cell receptor selection toward an HLA-B*3501-restricted human cytomegalovirus epitope”.

The full sequence used for the VDCU coding region is as follows:

(SEQ ID NO: 3)

Atggaaactctcctgggagtgtctttggtgattctatggcttcaactggc
tagggtgaacagtcaacagggagaagaggatcctcaggccttgagcatcc
aggagggtgaaaatgccaccatgaactgcagttacaaaactagtataaac
aatttacagtggtatagacaaaattcaggtagaggccttgtccacctaat
tttaatacgttcaaatgaaagagagaaacacagtggaagattaagagtca
cgcttgacacttccaagaaaagcagttccttgttgatcacggcttcccgg
gcagcagacactgcttcttacttctgtgctacgGAGgatagcaactatca
gttaatctggggcgctgggaccaagctaattataaagccaA

The homology arm sequences flanking the targeting construct are:

5′ Arm:

(SEQ ID NO: 4)

ggaacagagcagtgggtagctgcgaaagcttaaccccctctccaagcatg
cgtggaagcgcttgggcttctctgacagagaactgctgattttcatagtt
gagacttcaattcttagaagataatgtattttattattaatgtatataat
actaaatattatatcttttgctttagaagcatgtcacataggactgctta
ttatttgttcattgaaaaataaagaaatgggctggagaggtggctcagca
gttagtggcactcgctgctcttccataggatcttagttctagtactataa
gtagctcacaactgtctattactccagctgcaaggaatctgacagcctct
gcatccataagtacatggcattcactcacacataaataaaagaactctaa
aaaggaggaaaacaaacaaattggatagccagttctttgtaacttttcct
gggctgagtggtggtggtggtggtaattctgcaaattataaattttaaat
ggtattaatttagaactcactagcatctcttagttaataataactaagcc
agtctttctatttgaacttctttgctttaatgctgtttcatagaaggtac
acaaaattatatctatatgttaaggttcatatcctttggaattgagttat
ggatacaacagcaaccattaataaaggagaaagataagctttcactcatg
gattggtctgttgttaggacaggtgaagctcaactttcaccaagatcaac
cacattcatgatacctttgaagtttgtgtctccaattaaagatgctgaaa
gcttgctttgagtttcagctttatttactacagtcttgtttctcttgtaa
ccacccattcccatccccttcctaccaccccaaagtccttccaatcctgc
cacagccttccacatttacaaggcatcttctctgcttctgaacaccctgt
cctccctatcccttgagctgtcattcaagattcagctcaagagattcttt
ctaccaccaagttggctttatagataggttgttaggcctgtagaacccat
ttgctctgagatagtaagatgtttgttattttattagctaggattttggg
aattctggtccatttatttttaattgacaagcactcatcagacatttgca
gaataaatgaaaaaaaagcaataaaaaaaagccaatagtatagatcagtg
atagtaatctacaacagacaaaaggttagtactgacctctacaagaatca
actttcttagaaaaattgcaactctaaccaagattagagaggtctacttg
tttatgttattagggttttgctaatctggaatggcagattctacatgtgt
ttcaccagaagatggagaaagaaaagaatcaagtaggaaatagaaagaaa
agagaaattgtcaataaaacttaactatgtaaagtagctgatgatctcaa
ttaacatggatcataaagagaatgaaaagtgatttttctctgtagttatg
gaagcgatatcaaccataaaaattgatgtttgttgagaacttataaaatg
ccagacatcatatatggctcattatatttagctatccataaaactctatg
aactagaaattaatattgtttgtctgtgacattaaagaacaaagcttaga
cttaaatctgctgtttatgcacttagtgcacagttattagagtagacacg
agggacaaaattaccgaataggagcagcatgcatgtaagagagagaatac
tggagtaggaggaagaggaaccccttaggccatcctggggtgggaaatga
gagctaagtgtgctcagatgtctgtagagactacactattttttaaaagg
taaaaagaataggaataccaaatatccatcgataggagtttggataaact
agtttatgtagaccacaacgaacacaaatcagttaataaaaggaaatatg
tttctagatgtttttgttaaaatatggggcactggatacgatgtatggtc
tgactgtaaaacggatgctgtagtacaggcttcttctaggaggctgcaca
agctgtgcttagaggtttcccctggggcagctcaagggcagtaaggaca
3′ Arm:

(SEQ ID NO: 5)

ccaagagattggatcggagaataagcatgagtagttattgagatctgggt
ctgactgcaggtagcgtggtcttctagacgtttaagtgggagatttggag
gggatgaggaatgaaggaacttcaggatagaaaagggctgaagtcaagtt
cagctcctaaaatggatgtgggagcaaactttgaagataaactgaatgac
ccagaggatgaaacagcgcagatcaaagaggggcctggagctctgagaag
agaaggagactcatccgtgttgagtttccacaagtactgtcttgagtttt
gcaataaaagtgggatagcagagttgagtgagccgtaggctgagttctct
cttttgtctcctaagtttttatgactacaaaaatcagtagtatgtcctga
aataatcattaagctgtttgaaagtatgactgcttgccatgtagatacca
tggcttgctgaataatcagaagaggtgtgactcttattctaaaatttgtc
acaaaatgtcaaaatgagagactctgtaggaacgagtccttgacagacag
ctcaaggggtttttttcctttgtctcatttctacatgaaagtaaatttga
aatgatcttttttattataagagtagaaatacagttgggtttgaactata
tgttttaatggccacggttttgtaagacatttggtcctttgttttcccag
ttattactcgattgtaattttatatcgccagcaatggactgaaacggtcc
gcaacctcttctttacaactgggtgacctcgcggctgtgccagccatttg
gcgttcaccctgccgctaagggccatgtgaacccccgcggtagcatccct
tgctccgcgtggaccactttcctgaggcacagtgataggaacagagccac
taatctgaagagaacagagatgtgacagactacactaatgtgagaaaaac
aaggaaagggtgacttattggagatttcagaaataaaatgcatttattat
tatattcccttattttaattttctattagggaattagaaagggcataaac
tgctttatccagtgttatattaaaagcttaatgtatataatcttttagag
gtaaaatctacagccagcaaaagtcatggtaaatattctttgactgaact
ctcactaaactcctctaaattatatgtcatattaactggttaaattaata
taaatttgtgacatgaccttaactggttaggtaggatatttttcttcatg
caaaaatatgactaataataatttagcacaaaaatatttcccaatacttt
aattctgtgatagaaaaatgtttaactcagctactataatcccataattt
tgaaaactatttattagcttttgtgtttgacccttccctagccaaaggca
actatttaaggaccctttaaaactcttgaaactactttagagtcattaag
ttatttaaccacttttaattactttaaaatgatgtcaattcccttttaac
tattaatttattttaaggggggaaaggctgctcataattctattgttttt
cttggtaaagaactctcagttttcgtttttactacctctgtcacccaaga
gttggcatctcaacagaggggactttccgagaggccatctggcagttgct
taagatcagaagtgaagtctgccagttcctcccaggcaggtggcccagat
tacagttgacctgttctggtgtggctaaaaattgtcccatgtggttacaa
accattagaccagggtctgatgaattgctcagaatatttctggacaccca
aatacagaccctggcttaaggccctgtccatacagtaggtttagcttggc
tacaccaaaggaagccatacagaggctaatatcagagtattcttggaaga
gacaggagaaaatgaaagccagtttctgctcttaccttatgtgcttgtgt
tcagactcccaaacatcaggagtgtcagataaactggtctgaatctctgt
ctgaagcatggaactgaaaagaatgtagtttcagggaagaaaggcaatag
aaggaagcctgagaatatcttcaaagggtcagactcaatttactttctaa
agaagtagctaggaactagggaataacttagaaacaacaagattgtatat
atgtgcatcctggccccattgttccttatctgtagggataagcgtgcttt
tttgtgtgtctgtatataacataactgtttacacataatacactgaaatg
gagcccttccttgtt

### Example 3: Productive TCR Variable Region Gene Segment Rearrangement and Mutation from an Ectopic Genomic Location

Embryonic stem cell clones heterozygous for a modified IgH locus (depicted in FIGS. 1-2) were generated using the S-RMCE methods described previously. The following human TCRβ variable domain exons are therefore incorporated:
TCRBV19, TCRBV20-1, TCRBV24-1, TCRBV25-1, TCRBV27, TCRBV28, TCRBV29-1
TCRBD1
TCRBJ1-1, TCRBJ1-2, TCRBJ1-3, TCRBJ1-4, TCRBJ1-5, TCRBJ1-6

Blastocyst microinjection of ES cell clones was used to generate chimeric mice which possessed, in part, cells derived from the injected ES cells (i.e. cells heterozygous for the TCRβ-IgH chimeric locus). A single chimeric mouse was analysed using the methods described. The mouse was female and exhibited approximately 20% chimerism by coat colour. The animal was sacrificed at 6 weeks old and spleen and bone marrow tissue was collected.

We determined use of all of the V and J TCR exons that we provided at the Ig locus, TCR CDR3 lengths followed a normal distribution curve which resembled that of a human TCRβ CDR3 profile and we observed junctional diversity produced by nucleotide additions and deletions at recombined TCR gene segment junctions.

(A) Purified Pooled B or T Lymphocyte Populations from Naïve Mouse Immune Tissues

TABLE 1
Reagent details

| Reagent | Supplier | Cat # | Lot # |
|---|---|---|---|
| | Gibco | 14190-094 | 1762883 |
| ACK Lysis Buffer | Life | A10492-01 | 0000400419 |
| | Technologies | | |
| TruStain ™ FcX/Fc | Biolegend | 101320 | B200134 |
| blocker | | | |
| Anti-Rat beads | BD Bioscience | 552844 | 50068034 |
| 7-AAD | eBioscience | 00-6993-50 | E-0031-1639 |
| CD19-PB | Biolegend | 115523 | b203467 |
| CD3-FITC | Biolegend | 100204 | b207073 |
| CD11c APC-efluor780 | eBioscience | 47-0114-82 | E10192-1636 |
| Ly-6G (Gr-1) APC- | eBioscience | 47-5931-82 | E09970-1639 |
| efluor780 | | | |
| F4/80 APC-efluor780 | eBioscience | 47-4801-82 | 4300060 |
| Heat-inactivated Foetal | Gibco | 10270-106 | 41G6320K |
| Bovine Serum | | | |

FACS buffer (3% FBS in PBS) prepared by adding 15 ml heat inactivated FBS to 500 ml to PBS. Buffer filtered into a sterile 500 ml container.

Step 1: Cell Isolation from Tissues
1. Using a syringe/small bore needle/microdissection tools and a petri-dish, cells flushed from bone marrow with filtered PBS/3% FCS buffer
2. Spleen processed via homogenising through a 40 um filter and flushing through with PBS/FBS buffer

### Step 2: Red Cell Lysis

1. Cell suspensions spun down at 500 g for 10 minutes
2. Supernatant removed
3. Red blood cells lysed by re-suspending pellet, using a wide-bore tip, in 1 mL of ACK Lysis Buffer (Life Technologies A10492-01), incubated at RT for 2 minutes with gentle swirling
4. 9 ml PBS/FCS buffer added to stop reaction
5. 10 ml cell suspensions passed through 40 um strainers again. Old Falcon tubes rinsed with additional 10 mL of buffer, added to new falcon tube via strainer=20 ml final volume cell suspension per tissue.
6. Centrifuged, 500 g, 10 mins
7. Resuspended in 450 μl buffer
8. At this point, two 75 μl aliquots of cells (each aliquot equals ⅙ of total cells collected) were spun at 500 g for 10 mins, pellets were immediately lysed in 1 ml Trizol reagent.

### Step 3: Fc Block

1. 20 μl of Fc blocker added per sample, samples incubated for at least 10 mins at 4° C.
2. 20 μl aliquots of cells removed for “unstained control” and 7-AAD single stain controls, topped up to 240 μl with buffer
3. Remaining cell volume is ^{˜}300 μl

### Step 4: Antibody Staining

1. 100 μl of 4× concentration staining solution added to 300 μl of cells
2. Incubated at 4° C. for at least 20 minutes
3. 10 ml of buffer added to the samples and cell controls.
4. Cells spun at 500 g for 10 mins
5. Supernatant removed carefully using a 5 ml stripette without disturbing the cell pellet
6. Cells resuspended in remaining small volume of buffer
7. Filtered into FACS tube
8. Tube rinsed with additional 600 μl of buffer, filtered into FACS tube
9. FACS tube of stained cells ready for flow sorting

### Staining Controls

Control pool of cells at 240 μl, 100 μl volumes used for 7-AAD and unstained cells.

TABLE 2

| | | Species/ | Comp | Cell vol. | Staining |
|---|---|---|---|---|---|
| | | Isotype | beads/cells | (μL) | mix (μL) |
| 1 | Unstained Ctrl | / | Cells | 100.0 | / |
| 2 | 7-AAD | / | Cells | 100.0 | 1.0 |
| 3 | CD3-FITC | Rat IgG2a, k | Anti-rat comp | / | 0.5 |
| | | | beads | | |
| 4 | CD19-PB | Rat IgG2a, k | Anti-rat comp | / | 0.5 |
| | | | beads | | |
| 5 | Gr1-APC-efluor780 | Rat IgG2a, k | Anti-rat comp | / | 0.5 |
| | Cd11c-APC-efluor780 | Hamster IgG | beads | | |
| | F4/80-APC-eflu0r780 | Rat IgG2a, k | | | |

### FACS Gating Strategy

1. FSC vs SSC gate: Lymphocytes
2. Trigger pulse width vs SSC: singlets desired, doublets excluded
3. 7-AAD stain: live (unstained) cells selected
4. APC-efluor780: “dump” channel: unstained cells selected
5. CD19-PB vs CD3-FITC: single positive populations collected (CD19=B cells, CD3=T cells)

CD19+ or CD3+ cells were FACS sorted into 15 ml Falcon tubes. After collection, cells were spun down at 500 g for 10 minutes and cell pellets were lysed in 1 ml Trizol reagent.

### (b) RNA Extraction and Reverse Transcription

RNA was prepared from the following Trizol samples according to the manufacturer's recommendations.

TABLE 3

| | | | RNA yield |
|---|---|---|---|
| | | amount processed | (ng/μl) |
| 1 | whole spleen | ⅙ of total spleen cells | 227 |
| 2 | whole bone marrow | ⅙ of total BM cells | 382 |
| 3 | CD19+ B cells FACS sorted | 890,000 cells | 51 |
| | from bone marrow | | |

TABLE 4
RT reaction RNA sample volumes and amounts:

| | | RNA amount | total RT rxn |
|---|---|---|---|
| | | used in RT | volume (μl) |
| 1 | whole spleen | 2270 ng (10 μl) | 40 |
| 2 | whole bone marrow | 3820 ng (10 μl) | 40 |
| 3 | CD19+ B cells FACS sorted from BM | 510 ng (10 μl) | 20 |

TABLE 5
RT primers used:

| | |
|---|---|
| ELP1555 | ACACTCTTTCCCTACACGACGCTCTTCCGATCTNNNNNN |
| 8N P5 | NNATGGCCACCAGATTCTTATCAGAC (SEQ ID NO: 6) |
| AW078 IgD | ACACTCTTTCCCTACACGACGCTCTTCCGATCTNNNNNNN |
| 8N P5 | NCACTCTGAGAGGAGGAACATGT (SEQ ID NO: 7) |

Reverse transcription was performed using the Superscript III RT kit (Invitrogen).

TABLE 6
RT reaction volumes used per 20 μl reaction (note:
components doubled for 40 μl reactions):

| component | volume per 20 μl rxn (μl) |
|---|---|
| AW078 (IgD) 8N P5 primer (10 uM) | 1 |
| ELP1555 8N P5 primer (10 uM) | 1 |
| dNTPs | 1 |
| First Strand buffer | 2 |
| 0.1M DTT | 1 |
| RNAseOUT | 1 |
| Superscript III RT (Invitrogen) | 1 |
| sample RNA | 10 |
| H2O | to 20 μl |

RT Reaction protocol:
50° C. for 60 mins
70° C. for 15 mins

### Chilled on ice

1 μl of RNase A/T (Thermo Scientific, #EN0551) added to each RT reaction (20 or 40 μl).

Incubated on a thermal cycler, 37° C. for 15 minutes.

cDNA product immediately purified using magnetic beads (Beckman Coulter “Agencourt AMPure XP” Kit). Samples each eluted from beads into 15 μl H2O.

(c) PCR Amplification from cDNA to Generate Sequence Library

### 1^{st} Round PCR

All PCRs performed using Q5 High Fidelity PCR Mastermix (New England Biolabs).

Reaction conditions (25 μl):

TABLE 7

| | |
|---|---|
| cDNA template from RT step | 4.5 μl |
| 2X Q5 master mix | 12.5 μl |
| TCRBV RW P7 primer mix (7 primers, 10 μM | 1 μl |
| each primer) | |
| AW055 (partial P5 reverse primer), 10 uM | 1 μl |
| H2O | To 25 μl |

TABLE 8
Forward Primers

| | |
|---|---|
| TCRBV19RW P7 | GTGACTGGAGTTCAGACGTGTGCTCTTCCGATCTNNNNgatggtggaatcactcagtc (SEQ |
| | ID NO: 8) |
| TCRBV20-1RW P7 | GTGACTGGAGTTCAGACGTGTGCTCTTCCGATCTNNNNggtgctgtcgtctctcaaca (SEQ |
| | ID NO: 9) |
| TCRBV24-1 RW | GTGACTGGAGTTCAGACGTGTGCTCTTCCGATCTNNNNgatgctgatgttacccagac (SEQ |
| P7 | ID NO: 10) |
| TCRBV25-1 RW | GTGACTGGAGTTCAGACGTGTGCTCTTCCGATCTNNNNgaagctgacatctaccagac (SEQ |
| P7 | ID NO: 11) |
| TCRBV27 RW P7 | GTGACTGGAGTTCAGACGTGTGCTCTTCCGATCTNNNNgaagcccaagtgacccagaaccc |
| | (SEQ ID NO: 12) |
| TCRBV28 RW P7 | GTGACTGGAGTTCAGACGTGTGCTCTTCCGATCTNNNNgatgtgaaagtaacccagagctc |
| | (SEQ ID NO: 13) |
| TCRBV29-1 RW | GTGACTGGAGTTCAGACGTGTGCTCTTCCGATCTNNNNagtgctgtcatctctcaaaa (SEQ |
| P7 | ID NO: 14) |

TABLE 9
Reverse primer

| | |
|---|---|
| AW055 | acactctttccctacacgacg (SEQ ID NO: 15) |
| (partialP5) | |

PCR protocol used: Q5 Touchdown 68->60° C., 22 cycles in total.

Touch down PCR program:

$$98 ° C . 30 secs$$

$$…$$

$$98 ° C . 10 secs$$

$$68 ° C . to 60 ° C . 30 secs in 8 cycles$$

$$72 ° C . 20 secs$$

$$…$$

$$98 ° C . 10 secs$$

$$60 ° C . 30 secs \times 14 cycles$$

$$72 ° C . 20 secs$$

$$…$$

$$72 ° C . 2 min$$

Bead cleanup performed on all samples following PCR: 0.8 volumes of beads per sample i.e. 20 μl beads used for each 25 μl PCR reaction. Samples eluted into 15 μl H2O.

### 2^{nd} Round PCR

TABLE 10
PCR components

| | | P7 | | volume 1st | |
|---|---|---|---|---|---|
| | | barcode | P5 | round PCR | Oligos, |
| | | primer | primer | product used | 10 uM |
| | | used | used | (μl) | each (μl) |
| 1 | whole spleen | AW043 | AW042 | 4.5 | 1 + 1 |
| 2 | whole bone marrow | AW044 | AW042 | 4.5 | 1 + 1 |
| 3 | CD19+ B cells FACS | AW046 | AW042 | 4.5 | 1 + 1 |
| | sorted from BM | | | | |

12.5 μl Q5 Taq and 6 μl H₂O added per reaction to give 25 μl reaction volumes.

TABLE 11
Primers (NB: there is a phosphorothioate
bond between the terminal TT or GT)

| | |
|---|---|
| PE1 (P5) AW042 | AATGATACGGCGACCACCGAGATCTACACTCTTTCCCTACACGACGCTCTT |
| | (SEQ ID NO: 16) |
| PE2 index 1 - AW043 | CAAGCAGAAGACGGCATACGAGATCGTGATGTGACTGGAGTTCAGACGTG |
| | T (SEQ ID NO: 17) |
| PE2 index 2 - AW044 | CAAGCAGAAGACGGCATACGAGATACATCGGTGACTGGAGTTCAGACGTG |
| | T (SEQ ID NO: 18) |
| PE2 index 3 - AW045 | CAAGCAGAAGACGGCATACGAGATGCCTAAGTGACTGGAGTTCAGACGTG |
| | T (SEQ ID NO: 19) |
| PE2 index 4 - AW046 | CAAGCAGAAGACGGCATACGAGATTGGTCAGTGACTGGAGTTCAGACGTG |
| | T (SEQ ID NO: 20) |
| PE2 index 5 - AW047 | CAAGCAGAAGACGGCATACGAGATCACTGTGTGACTGGAGTTCAGACGTG |
| | T (SEQ ID NO: 21) |

### PCR Protocol Used:

$$98 ° C . 30 secs$$

$$…$$

$$98 ° C . 10 secs$$

$$60 ° C . 30 secs \times 30 cycles$$

$$72 ° C . 20 secs$$

$$…$$

$$72 ° C . 2 min$$

### (d) Gel Extraction and Quantification of PCR Products for Illumina MiSeq™ NGS Sequencing Run

15 μl of each PCR product was bead purified using 0.8 volumes (14.5 μl) beads, eluted into 10 μl water. Samples were then run on a 1.5% agarose gel.

Expected 2^{nd} round PCR product sizes were as follows:

^{˜}547 bp for TCRβ-IgMu

^{˜}511 bp for TCRβ-IgDelta

DNA bands corresponding to the expected product sizes from bead cleanup lanes (lying between 500 and 600 bp) were individually excised from the gel.

DNA Gel Extraction Kit spin columns (Millipore) were used according to protocol: gel slices were added to individual columns which were then centrifuged. Liquid DNA in gel buffer was collected, ^{˜}25 μl per sample.

18 μl of each sample was bead purified using 0.8 volumes (14.5 μl) beads, eluted into 10 μl Elution Buffer.

Samples were quantified using using KAPA Library Quantification Kit, Illumina Platforms (KAPA Biosystems).

TABLE 12

| | sample and | P5 primer | qPCR quantified | Used for |
|---|---|---|---|---|
| RNA origin | prep method | used | concn (nM) | sequencing? |
| whole spleen | 1.1 bead pure | AW043 | 3.2 | yes, diluted |
| | | | | 1.6-fold |
| whole bone marrow | 1.2 bead pure | AW044 | 1.8 | yes, undiluted |
| CD19+ B cells FACS sorted | 1.4 bead pure | AW046 | 1.9 | yes, undiluted |
| from bone marrow | | | | |

The samples indicated were mixed to give an equimolar library of ^{˜}2 nM for NGS sequencing.

The final sample library was prepared and loaded into an Illumina MySeq reagent cartridge according to manufacturer's protocol for a 2 nM library. An Illumina MySeq run was performed according to manufacturer's protocol.

### (e) Illumina MySeq Post-Run Sequence Analysis

Sequences were obtained from the MiSeq machine as a set of forward and reverse fastq files for each sample. These were processed by first trimming the sequences of low-quality bases using TrimGalore (http://www.bioinformatics.babraham.ac.uk/projects/trim_galore/). The forward and reverse reads were then paired using flash (https://ccb.jhu.edu/software/FLASH/), and converted to fasta using fastq_to_fasta (http://hannonlab.cshl.edu/fastx_toolkit/commandline.html). Sequences were then analysed using a custom java program to separate the sequences based on their UMI (Unique Molecular Identifier): the 8 random nucleotides of each sequence which were introduced via primers used at the RT stage. Sequences belonging to each UMI were in turn clustered using cd-hit-est (http://weizhongli-lab.org/cd-hit/) to cluster sequences which were at least 90% identical together, merging PCR duplicates possessing read errors. The most abundant sequences from each cluster were then collected together, and analysed using blastn (https://blast.ncbi.nlm.nih.gov) against the set of inserted human TCR V, J and D genes and the wild-type mouse IGHV, D, J genes. Each sequence was assigned the highest scoring V, D, J segment and the human TCR CDR3 region identified using the closest match to the left-hand motif TCTGT and right hand motifs TTTGG, TTCGGTT. Sequences with identical CDR3 were grouped together, and statistics calculated on this set.

### Results

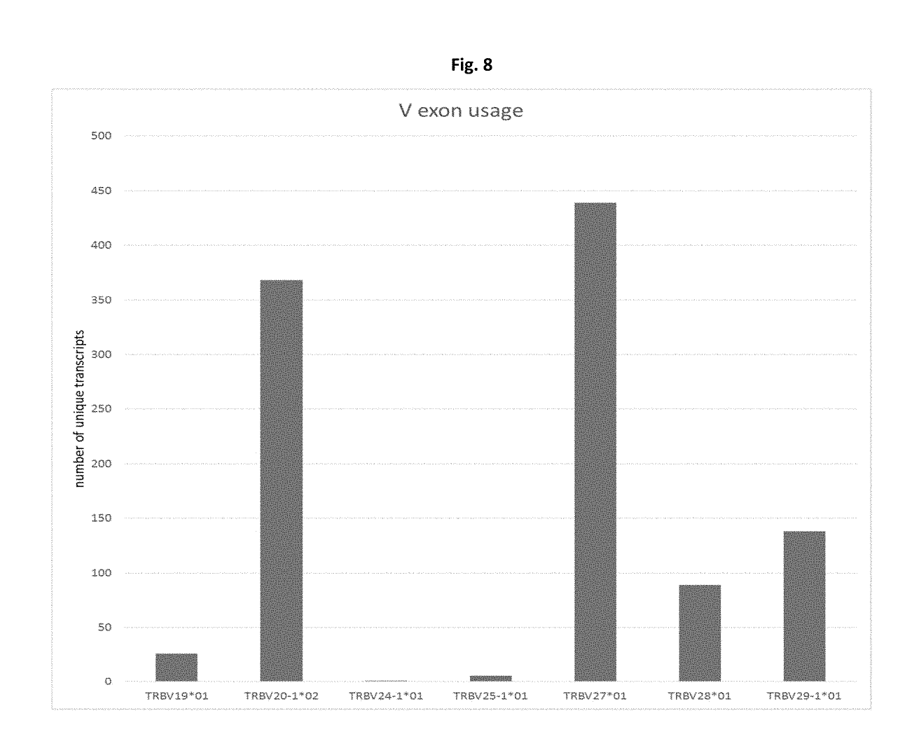

FIG. 8 shows Variable (V) gene segment usage among unique transcript sequences obtained from cellular RNA obtained from the “CD19+ B cells FACS sorted bone marrow” cell sample. Bars show numbers of unique sequences which aligned to each human TCRβ Variable gene segment.

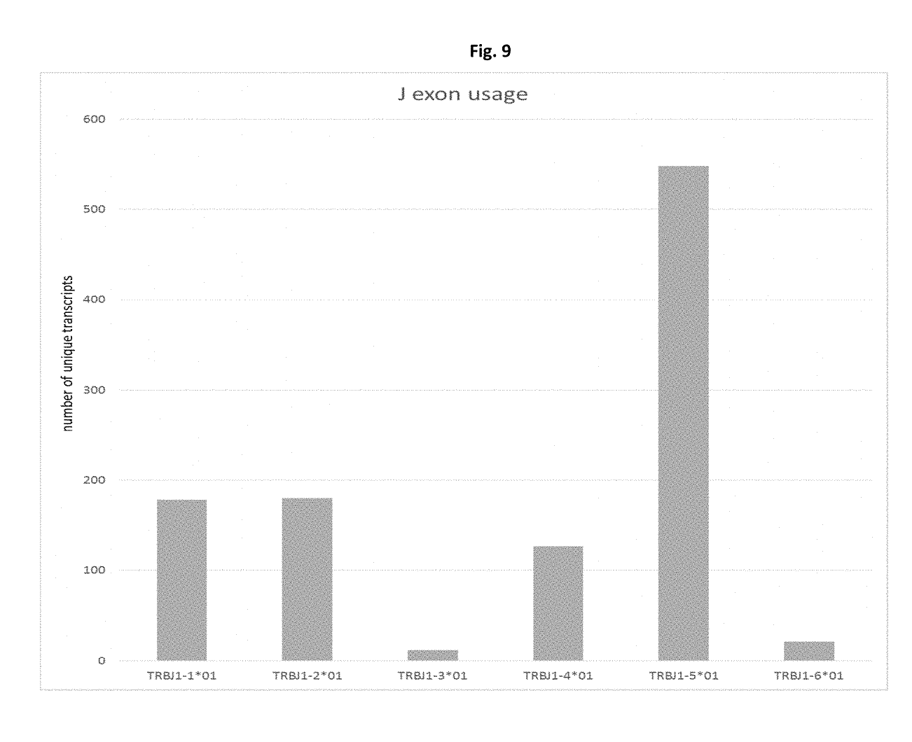

FIG. 9 shows Joining (J) gene segment usage among unique transcript sequences obtained from cellular RNA obtained from the “CD19+ B cells FACS sorted bone marrow” cell sample. Bars show numbers of unique sequences which aligned to each human TCRβ Joining gene segment.

Table 13 summarises the ten most common V-J exon pairings seen within productive rearranged transcripts.

TABLE 13

| | | | number of unique |
|---|---|---|---|
| rank | V exon | J exon | sequences |
| 1 | TRBV27*01 | TRBJ1-5*01 | 267 |
| 2 | TRBV27*01 | TRBJ1-1*01 | 104 |
| 3 | TRBV20-1*02 | TRBJ1-5*01 | 102 |
| 4 | TRBV20-1*02 | TRBJ1-2*01 | 100 |
| 5 | TRBV20-1*02 | TRBJ1-4*01 | 89 |
| 6 | TRBV29-1*01 | TRBJ1-5*01 | 83 |
| 7 | TRBV28*01 | TRBJ1-5*01 | 79 |
| 8 | TRBV20-1*02 | TRBJ1-1*01 | 63 |
| 9 | TRBV27*01 | TRBJ1-2*01 | 60 |
| 10 | TRBV29-1*01 | TRBJ1-4*01 | 32 |

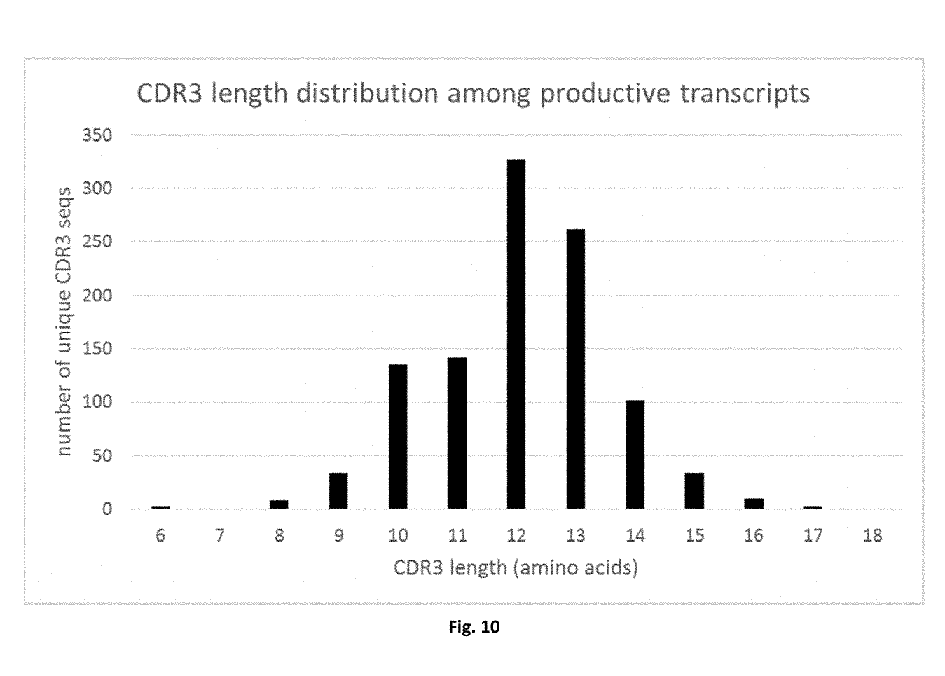

FIG. 10 shows CDR3 sequence length (in amino acids) among unique transcript sequences obtained from cellular RNA obtained from the “CD19+ B cells FACS sorted bone marrow” cell sample. Bars show numbers of unique sequences possessing CDR3 regions (defined as described in the methods section) of given lengths. CDR3 lengths followed a normal distribution curve which resembled that of a human TCRβ CDR3 profile (Blood. 2009 Nov. 5; 114(19): 4099-4107).

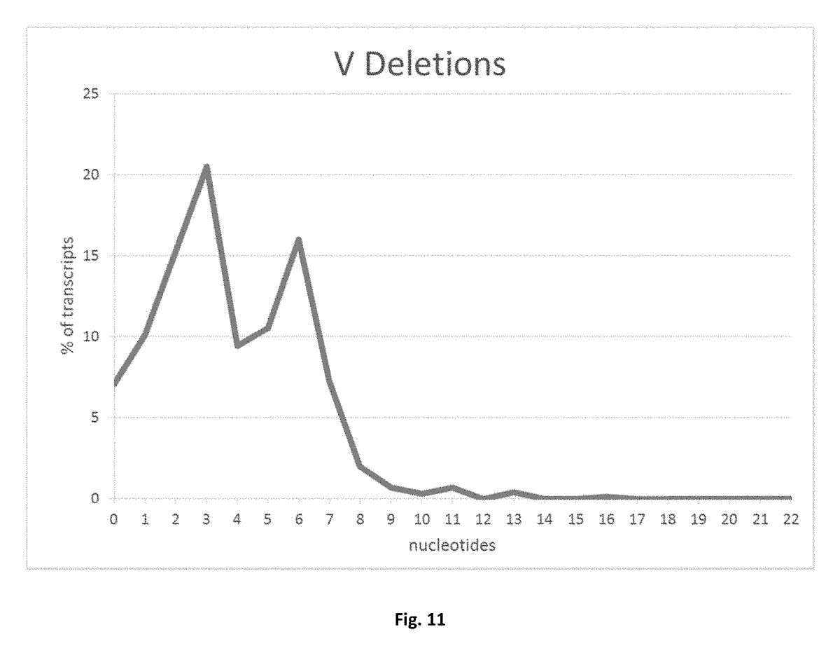

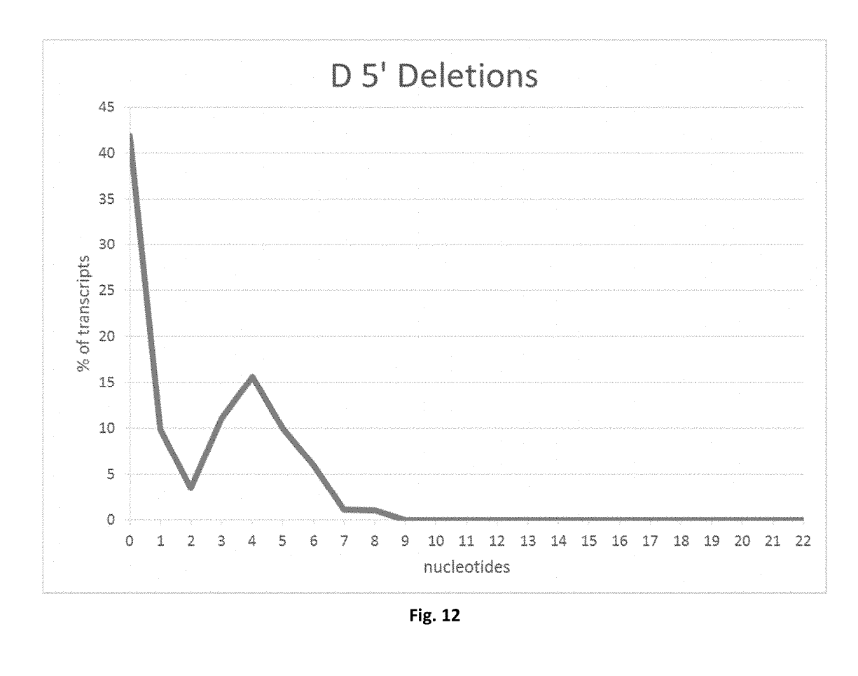

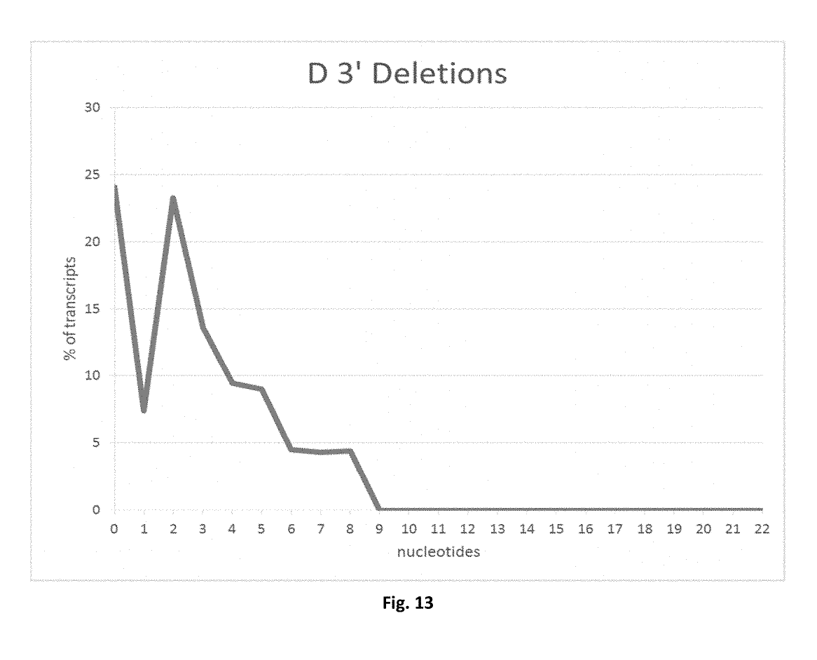

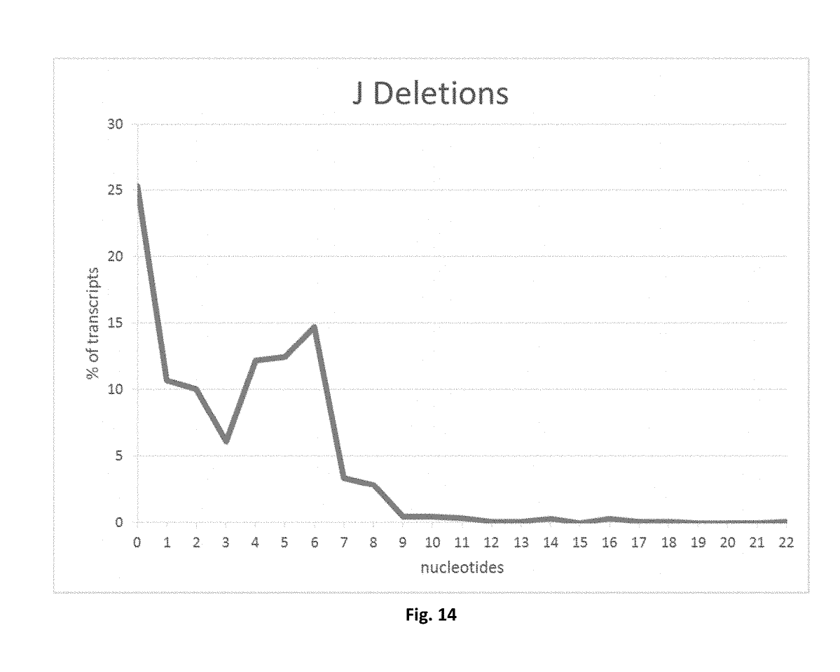

FIGS. 11-14 show gene segment nucleotide deletions occurring in unique transcript sequences obtained from cellular RNA obtained from the “CD19+ B cells FACS sorted bone marrow” cell sample. Nucleotide deletions were calculated by aligning each sequence to its closest matching V, D and J gene segment and adding up the number of missing/non-aligning nucleotides at each gene segment sequence end, compared with the original germline sequence.

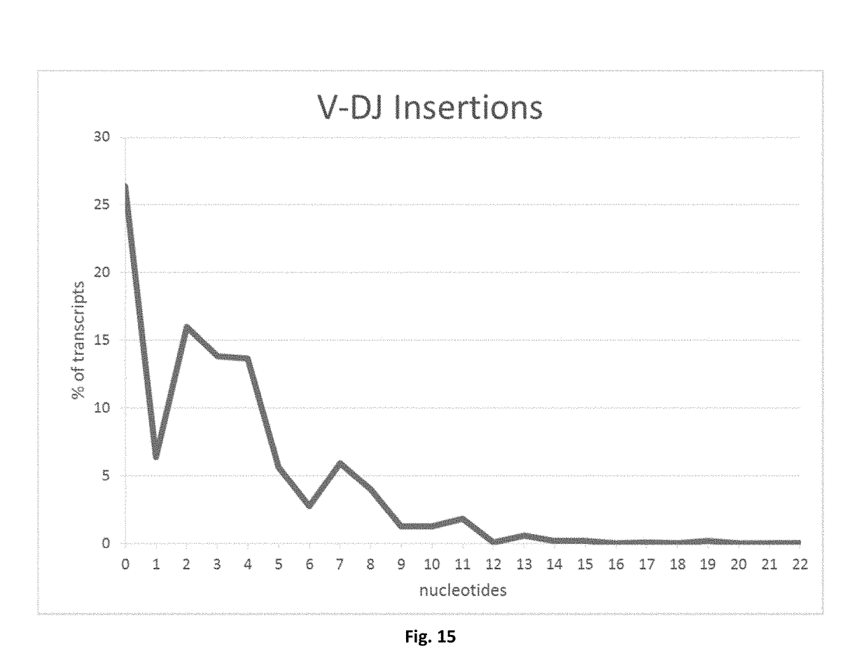

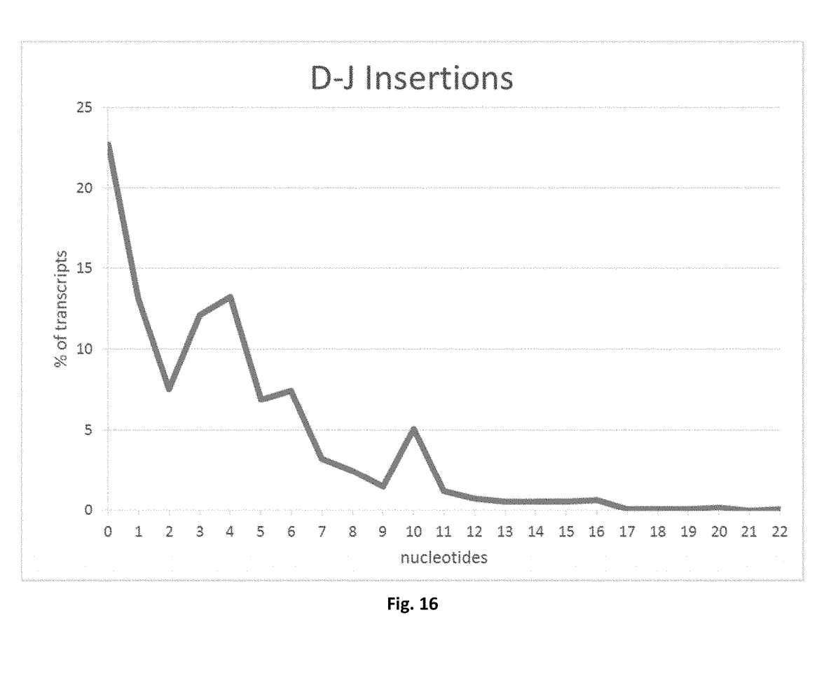

FIGS. 15 and 16 show gene segment nucleotide insertions occurring in unique transcript sequences obtained from cellular RNA obtained from the “CD19+ B cells FACS sorted bone marrow” cell sample. Nucleotide insertions were calculated by aligning each sequence to its closest matching V, D and J gene segment and adding up the number of non-aligning (i.e. additional non-germline encoded) nucleotides at each junction.

Table 14 summarises the deletion/insertion statistics represented in FIGS. 11-16.

TABLE 14

| number of base pairs | % of transcripts | | | | | |
|---|---|---|---|---|---|---|
| added/deleted | V deletions | J deletions | D 5′ deletions | D 3′ deletions | V-DJ insertions | D-J insertions |
| 0 | 7.0 | 25.3 | 41.9 | 24.1 | 26.4 | 22.7 |
| 1 | 10.1 | 10.7 | 9.8 | 7.4 | 6.4 | 13.1 |
| 2 | 15.2 | 10.0 | 3.5 | 23.2 | 16.0 | 7.5 |
| 3 | 20.5 | 6.1 | 11.2 | 13.6 | 13.8 | 12.1 |
| 4 | 9.4 | 12.2 | 15.6 | 9.5 | 13.6 | 13.2 |
| 5 | 10.5 | 12.5 | 9.9 | 9.0 | 5.6 | 6.8 |
| 6 | 16.0 | 14.7 | 6.0 | 4.5 | 2.7 | 7.4 |
| 7 | 7.2 | 3.4 | 1.1 | 4.3 | 5.9 | 3.2 |
| 8 | 2.0 | 2.8 | 1.0 | 4.4 | 3.9 | 2.4 |
| 9 | 0.7 | 0.5 | 0.0 | 0.0 | 1.2 | 1.5 |
| 10 | 0.3 | 0.5 | 0.0 | 0.0 | 1.2 | 5.1 |
| 11 | 0.7 | 0.4 | 0.0 | 0.0 | 1.8 | 1.2 |
| 12 | 0.0 | 0.1 | 0.0 | 0.0 | 0.1 | 0.8 |
| 13 | 0.4 | 0.1 | 0.0 | 0.0 | 0.6 | 0.6 |
| 14 | 0.0 | 0.3 | 0.0 | 0.0 | 0.2 | 0.6 |
| 15 | 0.0 | 0.0 | 0.0 | 0.0 | 0.2 | 0.6 |
| 16 | 0.1 | 0.3 | 0.0 | 0.0 | 0.0 | 0.7 |
| 17 | 0.0 | 0.1 | 0.0 | 0.0 | 0.1 | 0.1 |
| 18 | 0.0 | 0.1 | 0.0 | 0.0 | 0.0 | 0.1 |
| 19 | 0.0 | 0.0 | 0.0 | 0.0 | 0.2 | 0.1 |
| 20 | 0.0 | 0.0 | 0.0 | 0.0 | 0.0 | 0.2 |
| 21 | 0.0 | 0.0 | 0.0 | 0.0 | 0.0 | 0.0 |
| 22 | 0.0 | 0.1 | 0.0 | 0.0 | 0.0 | 0.1 |

### SUMMARY

As demonstrated in FIGS. 8-16, TCRβ-Cμ chimeric transcript sequences have been identified which have undergone the diversity-generating processes typical for IgH Cμ transcripts, namely D-J and V-DJ recombination and the use of multiple gene segments, nucleotide additions (presumably involving mouse terminal deoxynucleotidyl transferase activity (TdT)) and deletions within the sequence's CDR3 region.

Key findings:
Human TCRβ BAC insert V, D and J coding exons were identified as present in rearranged, in-frame transcripts in B cells
   Novel evidence that human TCR exons are able to rearrange at an ectopic (ie, non-naturally-occurring) genomic position, in this case at an Ig locus;
   Novel evidence that rearranged human TCR exons are able to be transcribed from an ectopic genomic position;
   The rearranged TCR V regions are used to and produce in-frame transcripts, spliced to a nucleotide sequence encoding an Ig constant region (in this example a Cμ region);
   Indicates mouse Ig regulatory elements are able to drive expression of transcripts comprising TCR variable regions;
   Dominant usage of some exons and lower usage of others; this may usefully reflect that dominance of certain V and J exons occur at the native human TCRβ locus;
   The TCRβ variable region is advantageously being acted on by orthologous factors; for example, (i) mouse factors (eg, mouse TdT) operating on human TCR sequence, and (ii) our hypothesis that IgH control may be acting on the TCR sequence at the Ig locus;
   Sequences show evidence of nucleotide addition/deletions at V-to-D and D-to-J junctions of TCR-Ig, thus generating CDR3 diversity as occurs during VDJ recombination at the native mouse IgH locus; and
   Human TCR-like CDR3 length data indicates potential for TCR-like binding preferences.

1. A non-human vertebrate comprising in its germline a locus for producing a plurality of antigen binding ligands, wherein each ligand comprises a polypeptide comprising a T-cell receptor (TCR) variable domain and an antibody constant domain, the ligand comprising an antigen binding site wherein the binding site comprises the variable domain, wherein the locus comprises
a. a T-cell receptor (TCR) variable region comprising in 5′ to 3′ direction one or more TCR V gene segments; optionally one or more D gene segments; and one or more J gene segments, wherein the variable region is capable of rearranging to produce a rearranged VDJ or VJ; and
b. an antibody constant region comprising one or more antibody C gene segments;
wherein the variable region is operably linked upstream of the antibody constant region whereby B-cells of the vertebrate express said polypeptides each comprising a TCR variable domain and an antibody constant domain.

2. The vertebrate of claim 1, wherein the antibody C gene segment(s) are endogenous segments of the vertebrate, optionally wherein the constant region is an endogenous heavy chain constant region at an endogenous heavy chain locus, or an endogenous light chain constant region at an endogenous light chain locus.

3. The vertebrate of claim 1, the locus comprising (i) the functional TCRBV, D and J gene segments of a human TCRβ locus from TCRBV19 to TCRBJ1-1 inclusive, and optionally up to TCRBJ1-6; or (ii) the functional TCRAV and J gene segments of a human TCRα locus from TCRAV24 to TCRAJ61 inclusive, and optionally up to TCRAJ1.

4. The vertebrate of claim 1, wherein
a. the one or more V gene segments are TCRAV segments and the one or more J gene segments are TCRAJ gene segments;
b. the one or more V gene segments are TCRBV segments and the one or more J gene segments are TCRBJ gene segments;
c. the one or more V gene segments are TCRCV segments and the one or more J gene segments are TCRCJ gene segments; or
d. the one or more V gene segments are TCRDV segments and the one or more J gene segments are TCRDJ gene segments.

5. The vertebrate of claim 1, wherein alternatively the variable region comprises a rearranged TCR VJ or VDJ.

6. The vertebrate of claim 1, wherein the locus comprises a human, mouse or rat antibody locus intronic enhancer between the variable and constant regions and/or a human, mouse or rat antibody locus 3′ enhancer operably linked downstream of said constant region.

7. The vertebrate of claim 1, wherein the constant region comprises the endogenous antibody heavy chain locus Eμ and Cμ of the vertebrate, optionally wherein the constant region comprises the DNA sequence of the endogenous Eμ through to (and including) the Cμ t of the vertebrate.

8. The vertebrate of claim 7, wherein the constant region comprises the endogenous antibody heavy chain locus mu switch sequence (Sμ) of the vertebrate, the constant region comprising downstream of the Cμ t a second switch sequence and a second C segment, wherein the constant region is capable of class-switch recombination (CSR) between the switches for isotype switching from the Cμ t to the second constant region gene segment and somatic hypermutation (SHM) of the TCR variable region.

9. The vertebrate of claim 1, wherein the constant region comprises
a. a first antibody C segment operably linked to a first switch sequence;
b. a second antibody C segment operably linked to a second switch sequence;
c. wherein the constant region is capable of CSR between the switches for isotype switching from the first to the second C segment and SHM of the TCR variable region.

10. The vertebrate of claim 8, wherein the second C segment is a human or a mouse gamma C.

11. The vertebrate of claim 1, comprising an endogenous activation induced cytidine deaminase (AID) nucleotide sequence that is capable of expressing AID for SHM of the TCR variable region.

12. The vertebrate of claim 1, comprising a first said locus and a second said locus, wherein
a. the TCR variable region of the first locus comprises one or more TCRAV segments and one or more TCRAJ gene segments and optionally the one or more antibody C gene segments are kappa C segments; and the TCR variable region of the second locus comprises one or more TCRBV segments, one or more TCRBD segments and one or more TCRBJ gene segments and optionally the one or more antibody C gene segments are heavy chain C segments, wherein the antigen binding site of each ligand comprises a TCR Vα domain and a TCR Vβ domain and optionally paired antibody heavy and kappa C domains; or
b. the TCR variable region of the first locus comprises one or more TCRGV segments and one or more TCRGJ gene segments and optionally the one or more antibody C gene segments are kappa C segments; and the TCR variable region of the second locus comprises one or more TCRDV segments, one or more TCRDD segments and one or more TCRDJ gene segments and optionally the one or more antibody C gene segments are heavy chain C segments, wherein the antigen binding site of each ligand comprises a TCR Vγ domain and a TCR Vδ domain and optionally paired antibody heavy and kappa C domains.

13. The vertebrate of claim 1, wherein the V and J gene segments are human gene segments, optionally wherein the antibody C gene segments are human, rat or mouse gene segments.

14. The vertebrate of claim 1, wherein the variable region is not at an endogenous antibody locus.

15. The vertebrate of claim 1, wherein the vertebrate is obtainable by
a. providing an embryonic stem cell of the vertebrate species (eg, mouse or rat);
b. inserting DNA comprising said TCR variable region gene segments into the ES cell genome in one or several steps to produce an ES cell product whose genome comprises the inserted variable region DNA operably linked upstream of the antibody constant region for expression of said polypeptides; and
c. developing said vertebrate from said product ES cell or a progeny thereof;
wherein either
d. the TCR variable region DNA is inserted into an endogenous antibody locus of the genome and the constant region comprises one or more C gene segments of the endogenous locus, wherein the insertion produces an engineered locus that is capable of expressing said polypeptides and CSR and SHM of the variable region; or
e. the TCR variable region DNA is comprised by a transgene, wherein the transgene comprises said constant region, wherein the transgene is inserted into said genome to provide a transgene locus that is capable of expressing said polypeptides and CSR and SHM of the variable region.

16. The vertebrate of claim 15, wherein the insertion in step (d) of TCR variable region DNA is an insertion (i) immediately 5′ of the 5′-end of the intron of said endogenous antibody locus; or (ii) between said 5′ end and the intronic enhancer of the intron.

17. The vertebrate of claim 16, wherein (i) the engineered locus comprises less than the complete intronic sequence immediately 5′ of said intronic enhancer found in wild-type vertebrates of said species; and/or (ii) the distance between the last inserted human J gene segment and said intronic enhancer is not >1 kb more than the distance between the last antibody J gene segment and the enhancer found in wild-type vertebrates of said species; and/or (iii) the inserted DNA comprises a 3′-most TCR J gene segment, wherein the segment is immediately 5′ of a further nucleotide sequence, wherein the further sequence is intron sequence that is naturally contiguous with said TCR J segment and the further sequence is no more than 1 kb in length.

18. A non-human vertebrate that is a progeny of the vertebrate developed in step (c) of claim 15.

19. The vertebrate of claim 1, wherein the vertebrate is incapable of antibody heavy chain and/or kappa chain variable region expression.

20. The vertebrate of claim 1, wherein the vertebrate is incapable of non-human vertebrate (i) TCR Vβ domain and/or TCR Vα domain expression; (ii) TCR Vδ domain and/or TCR Vγ domain expression; (iii) TCR Vβ and TCR Vδ domain expression or (iv) TCR Vβ, Vα, Vδ and Vγ domain expression.

21. The vertebrate of claim 1, wherein the vertebrate comprises antigen presenting cells comprising nucleic acid for surface expressing a peptide antigen receptor comprising a human MHC protein, wherein the vertebrate is capable of producing said plurality of ligands when the vertebrate is immunized with a peptide-MHC antigen (pMHC) comprising said human MHC protein.

22. The vertebrate of claim 21, wherein antigen presenting cells further comprise nucleic acid for surface expressing human beta-2 microglobulin complexed with the MHC protein, wherein the MHC protein is a human class I MHC protein.

23. A non-human vertebrate embryo which is capable of developing into a vertebrate of claim 1.

24. An isolated B-cell; thymus cell or tissue; spleen cell or tissue; bone marrow cell or tissue; ES cell; or iPS cell obtainable from a vertebrate of claim 1.

25. A method of producing one or more polypeptides, wherein each polypeptide comprises a T-cell receptor (TCR) variable domain and an antibody constant domain, the method comprising
a. providing a vertebrate according to claim 1;
b. immunizing the vertebrate with an antigen to effect CSR and SHM in the vertebrate, whereby a plurality of polypeptides comprising affinity matured TCR V domains and antibody constant domains are expressed by B-cells of the vertebrate; and
c. selecting one or more B-cells capable of expressing a said polypeptide; selecting one or more of said polypeptides; isolating one or more affinity matured TCR V domains thereof; or isolating one or more nucleotide sequences each encoding an affinity matured TCR V domain of a said expressed polypeptide;
d. wherein the polypeptide(s) or TCR V domain(s) of (c) specifically binds to the antigen; and
e. optionally expressing said polypeptide(s) or TCR V domain(s).

26. (canceled)

27. A method of using a non-human vertebrate to select for an affinity matured TCR variable domain or a nucleotide sequence encoding an affinity matured TCR variable domain, wherein the variable domain is capable of expression in vivo in a vertebrate, the method comprising
a. providing a non-human vertebrate wherein at least one antibody heavy chain locus of the vertebrate comprises a first TCR variable region upstream of the antibody heavy chain constant region for expressing first mRNA transcripts encoding polypeptides comprising an affinity matured TCR V domain and an antibody C domain; at least one antibody light chain locus of the vertebrate comprises a second TCR variable region upstream of the antibody light chain locus for expressing second mRNA transcripts encoding polypeptides comprising a TCR V domain and an antibody C domain;
b. immunizing the vertebrate with an antigen to effect CSR and SHM in the vertebrate, whereby a plurality of antigen-specific TCR-Ig ligands comprising affinity matured TCR V domains are expressed by B-cells of the vertebrate; and
c. selecting one or more B-cells capable of expressing a said TCR-Ig; selecting one or more of said TCR-Ig; isolating one or more affinity matured TCR V domains thereof;
or isolating one or more nucleic acid sequences each encoding an affinity matured TCR V domain of an expressed TCR-Ig;
d. wherein the TCR-Ig(s) or TCR V domain(s) of (c) specifically bind to the antigen; and
e. optionally expressing the one or more TCR-Igs or one or more TCR V domains.

28-34. (canceled)

35. A multispecific antigen-binding ligand obtainable by the method of claim 25.

36. A plurality of B-cells or hybridoma cells that express a plurality of different affinity matured TCR variable domains, wherein one or more of the variable domains specifically binds to an antigen.

37. (canceled)

38. A plurality of mammalian cells that express a plurality of at least 10 different affinity matured TCR variable domains, wherein one or more of the TCR variable domains specifically binds to an antigen.

39-44. (canceled)

45. The vertebrate of claim 1, wherein each ligand has the structure of an antibody except wherein the ligand comprises an affinity matured TCR variable domain instead of an antibody variable domain, or wherein the polypeptide or V domain is comprised by such a ligand.

46. The vertebrate of claim 1, wherein the vertebrate is a rodent (eg, a mouse or a rat).

47. The vertebrate of claim 46, wherein the mouse is a 129 or C57BL/6 strain mouse.

48. A TCRV-Ig comprising a TCR variable domain obtained or obtainable by the method of claim 25, wherein the TCRV-Ig specifically binds to pMHC antigen.

49. The TCRV-Ig of claim 48 comprising (in N- to C-terminal direction) said TCR V domain directly fused to a human antibody constant domain.

50. A TCR variable domain obtained or obtainable by the method of claim 25, wherein the TCR V domain specifically binds to pMHC antigen.

51-57. (canceled)

58. A method for treating or preventing a cancer or an autoimmune disease or condition in a patient, comprising administering to the patient the TCRV-Ig, of claim 48, wherein the antigen comprises an epitope of a tumour-associated antigen (TAA) or an immune checkpoint target.

59. The TCRV-Ig claim 48, wherein the TCRV-Ig binds said pMHC antigen with a binding affinity (KD) of less than 100 nM as determined by surface plasmon resonance (SPR).

60. A nucleic acid comprising a nucleotide sequence encoding the TCRV-Ig of claim 48, optionally comprised by an expression vector for expressing the TCRV-Ig.

61. An engineered immune cell comprising a TCRV-Ig of claim 48, wherein the immune cell expresses the TCRV-Ig.

62. The cell of claim 61, wherein the cell is a B-cell, T-cell, NK cell or TIL (tumour infiltrating lymphocyte).

63. A CAR-T cell comprising a chimaeric antigen receptor (CAR), the receptor comprising an extracellular moiety, a transmembrane moiety and an intracellular signalling moiety, wherein the extracellular moiety comprises the TCRV-Ig of claim 48 for expressing the TCRV-Ig as part of the extracellular moiety of the receptor.

64. A method for treating or preventing a disease or condition in a patient, comprising administering to the patient the cell of claim 61, wherein the cell is autologous to the patient, or an allogeneic cell from a donor of the same species as the patient.

65. A method of treating or preventing a disease or condition in a human patient, comprising administering to the patient the TCRV-Ig of claim 48, wherein said pMHC antigen comprises an MHC protein sequence that is matched with MHC of the patient.

66. A nucleic acid comprising a nucleotide sequence encoding the CAR recited in claim 63.

67. A method of identifying an antigen, the method comprising
a. carrying out the method of claim 25 to select a nucleotide sequence encoding an affinity matured TCR V domain, wherein the antigen is a cell surface-expressed antigen;
b. using the selected sequence to produce a second nucleotide sequence encoding a ligand comprising an antigen binding site, wherein the binding site comprises a said affinity matured TCR V domain and binds to the antigen with a binding affinity (KD) of less than 100 nM as determined by surface plasmon resonance (SPR);
c. using the second sequence to express copies of the ligand;
d. providing a plurality of cells comprising cell surface-expressed epitopes;
e. exposing the plurality of cells to copies of said ligand;
f. selecting one or more cells that are specifically bound by ligand; and
g. identifying the cell surface antigen of a said cell that is specifically bound by a ligand; and
h. optionally expressing the antigen or producing nucleic acid comprising nucleotide sequence encoding the antigen.

68-70. (canceled)

71. A method of transcribing a rearranged TCR variable region sequence in a non-human vertebrate or cell, the method comprising ectopically transcribing said rearranged TCR variable region sequence in said vertebrate or cell to produce mRNA transcripts encoding a TCR V domain and optionally an antibody constant domain.

72. A method of rearranging TCR V, D and J, or V and J gene segments to produce a rearranged TCR variable region sequence in a non-human vertebrate or cell, the method comprising ectopically rearranging said gene segments in said vertebrate or cell, whereby a transcribable rearranged TCR variable region sequence is produced.

73-76. (canceled)

77. A non-human vertebrate or non-human vertebrate cell that comprises a rearranged TCR V region that is expressible to produce one or more in-frame transcripts comprising a TCR V region nucleotide sequence spliced to a nucleotide sequence encoding an Ig constant region.

78. The vertebrate of claim 1, wherein said vertebrate expresses a plurality of different rearranged TCR VDJ, wherein each VDJ is the product of rearrangement of a V, D and J, wherein the V/J are selected from the group consisting of TCRBV27/TCRBJ1-5, TCRBV27/TCRBJ1-1, TCRBV20-1/TCRBJ1-5, TCRBV20-1/TCRBJ1-2, TCRBV20-1/TCRBJ1-4, TCRBV29-1/TCRBJ1-5, TCRBV28/TCRBJ1-5, TCRBV20-1/TCRBJ1-1, TCRBV27/TCRBJ1-2 and TCRBV29-1/TCRBJ1-4.

79. A plurality of B-cells comprising one or more immunoglobulin loci that comprise recombined TCR variable regions, wherein the variable regions comprise TCR gene segment junctional mutation.

80-106. (canceled)

107. A non-human vertebrate or a non-human vertebrate cell that comprises a rearranged TCRB variable region that is ectopically positioned in the genome of the vertebrate of cell, wherein the vertebrate or cell expresses TCRB V domains comprising most commonly a CDR3 length of 11, 12 or 13 amino acids.

108-109. (canceled)


SEQUENCE LISTING (21 sequences)

SEQ ID NO: 1 - DNA, 70 bp, Homo sapiens
taatcagagtatctacagcgctcctggagtaacccaaaccttatggttgctggaaagataaaaaaccaat

SEQ ID NO: 2 - DNA, 70 bp, Homo sapiens
cagcttgttgctctgctgtgttgcctgcagttcctcagctgtagagctccttgcttagtcttcagggctg

SEQ ID NO: 3 - DNA, 391 bp, Homo sapiens
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggaggatagcaactatcagttaatctggggcgctgggaccaagctaattataaagccaa

SEQ ID NO: 4 - DNA, 2099 bp, Mus musculus
ggaacagagcagtgggtagctgcgaaagcttaaccccctctccaagcatgcgtggaagcgcttgggcttctctgacagagaactgctgattttcatagttgagacttcaattcttagaagataatgtattttattattaatgtatataatactaaatattatatcttttgctttagaagcatgtcacataggactgcttattatttgttcattgaaaaataaagaaatgggctggagaggtggctcagcagttagtggcactcgctgctcttccataggatcttagttctagtactataagtagctcacaactgtctattactccagctgcaaggaatctgacagcctctgcatccataagtacatggcattcactcacacataaataaaagaactctaaaaaggaggaaaacaaacaaattggatagccagttctttgtaacttttcctgggctgagtggtggtggtggtggtaattctgcaaattataaattttaaatggtattaatttagaactcactagcatctcttagttaataataactaagccagtctttctatttgaacttctttgctttaatgctgtttcatagaaggtacacaaaattatatctatatgttaaggttcatatcctttggaattgagttatggatacaacagcaaccattaataaaggagaaagataagctttcactcatggattggtctgttgttaggacaggtgaagctcaactttcaccaagatcaaccacattcatgatacctttgaagtttgtgtctccaattaaagatgctgaaagcttgctttgagtttcagctttatttactacagtcttgtttctcttgtaaccacccattcccatccccttcctaccaccccaaagtccttccaatcctgccacagccttccacatttacaaggcatcttctctgcttctgaacaccctgtcctccctatcccttgagctgtcattcaagattcagctcaagagattctttctaccaccaagttggctttatagataggttgttaggcctgtagaacccatttgctctgagatagtaagatgtttgttattttattagctaggattttgggaattctggtccatttatttttaattgacaagcactcatcagacatttgcagaataaatgaaaaaaaagcaataaaaaaaagccaatagtatagatcagtgatagtaatctacaacagacaaaaggttagtactgacctctacaagaatcaactttcttagaaaaattgcaactctaaccaagattagagaggtctacttgtttatgttattagggttttgctaatctggaatggcagattctacatgtgtttcaccagaagatggagaaagaaaagaatcaagtaggaaatagaaagaaaagagaaattgtcaataaaacttaactatgtaaagtagctgatgatctcaattaacatggatcataaagagaatgaaaagtgatttttctctgtagttatggaagcgatatcaaccataaaaattgatgtttgttgagaacttataaaatgccagacatcatatatggctcattatatttagctatccataaaactctatgaactagaaattaatattgtttgtctgtgacattaaagaacaaagcttagacttaaatctgctgtttatgcacttagtgcacagttattagagtagacacgagggacaaaattaccgaataggagcagcatgcatgtaagagagagaatactggagtaggaggaagaggaaccccttaggccatcctggggtgggaaatgagagctaagtgtgctcagatgtctgtagagactacactattttttaaaaggtaaaaagaataggaataccaaatatccatcgataggagtttggataaactagtttatgtagaccacaacgaacacaaatcagttaataaaaggaaatatgtttctagatgtttttgttaaaatatggggcactggatacgatgtatggtctgactgtaaaacggatgctgtagtacaggcttcttctaggaggctgcacaagctgtgcttagaggtttcccctggggcagctcaagggcagtaaggaca

SEQ ID NO: 5 - DNA, 2265 bp, Mus musculus
ccaagagattggatcggagaataagcatgagtagttattgagatctgggtctgactgcaggtagcgtggtcttctagacgtttaagtgggagatttggaggggatgaggaatgaaggaacttcaggatagaaaagggctgaagtcaagttcagctcctaaaatggatgtgggagcaaactttgaagataaactgaatgacccagaggatgaaacagcgcagatcaaagaggggcctggagctctgagaagagaaggagactcatccgtgttgagtttccacaagtactgtcttgagttttgcaataaaagtgggatagcagagttgagtgagccgtaggctgagttctctcttttgtctcctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccgcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactataatcccataattttgaaaactatttattagcttttgtgtttgacccttccctagccaaaggcaactatttaaggaccctttaaaactcttgaaactactttagagtcattaagttatttaaccacttttaattactttaaaatgatgtcaattcccttttaactattaatttattttaaggggggaaaggctgctcataattctattgtttttcttggtaaagaactctcagttttcgtttttactacctctgtcacccaagagttggcatctcaacagaggggactttccgagaggccatctggcagttgcttaagatcagaagtgaagtctgccagttcctcccaggcaggtggcccagattacagttgacctgttctggtgtggctaaaaattgtcccatgtggttacaaaccattagaccagggtctgatgaattgctcagaatatttctggacacccaaatacagaccctggcttaaggccctgtccatacagtaggtttagcttggctacaccaaaggaagccatacagaggctaatatcagagtattcttggaagagacaggagaaaatgaaagccagtttctgctcttaccttatgtgcttgtgttcagactcccaaacatcaggagtgtcagataaactggtctgaatctctgtctgaagcatggaactgaaaagaatgtagtttcagggaagaaaggcaatagaaggaagcctgagaatatcttcaaagggtcagactcaatttactttctaaagaagtagctaggaactagggaataacttagaaacaacaagattgtatatatgtgcatcctggccccattgttccttatctgtagggataagcgtgcttttttgtgtgtctgtatataacataactgtttacacataatacactgaaatggagcccttccttgtt

SEQ ID NO: 6 - DNA, 65 bp, Artificial Sequence
  Primer ELP1555 8N P5
  misc_feature (34)..(41): n is a, c, g, or t
acactctttccctacacgacgctcttccgatctnnnnnnnnatggccaccagattcttatcagac

SEQ ID NO: 7 - DNA, 63 bp, Artificial Sequence
  Primer AW078 IgD 8N P5
  misc_feature (34)..(41): n is a, c, g, or t
acactctttccctacacgacgctcttccgatctnnnnnnnncactctgagaggaggaacatgt

SEQ ID NO: 8 - DNA, 58 bp, Artificial Sequence
  Primer TCRBV19RW P7
  misc_feature (35)..(38): n is a, c, g, or t
gtgactggagttcagacgtgtgctcttccgatctnnnngatggtggaatcactcagtc

SEQ ID NO: 9 - DNA, 58 bp, Artificial Sequence
  Primer TCRBV20-1RW P7
  misc_feature (35)..(38): n is a, c, g, or t
gtgactggagttcagacgtgtgctcttccgatctnnnnggtgctgtcgtctctcaaca

SEQ ID NO: 10 - DNA, 58 bp, Artificial Sequence
  Primer TCRBV24-1 RW P7
  misc_feature (35)..(38): n is a, c, g, or t
gtgactggagttcagacgtgtgctcttccgatctnnnngatgctgatgttacccagac

SEQ ID NO: 11 - DNA, 58 bp, Artificial Sequence
  Primer TCRBV25-1 RW P7
  misc_feature (35)..(38): n is a, c, g, or t
gtgactggagttcagacgtgtgctcttccgatctnnnngaagctgacatctaccagac

SEQ ID NO: 12 - DNA, 61 bp, Artificial Sequence
  Primer TCRBV27 RW P7
  misc_feature (35)..(38): n is a, c, g, or t
gtgactggagttcagacgtgtgctcttccgatctnnnngaagcccaagtgacccagaaccc

SEQ ID NO: 13 - DNA, 61 bp, Artificial Sequence
  Primer TCRBV28 RW P7
  misc_feature (35)..(38): n is a, c, g, or t
gtgactggagttcagacgtgtgctcttccgatctnnnngatgtgaaagtaacccagagctc

SEQ ID NO: 14 - DNA, 58 bp, Artificial Sequence
  Primer TCRBV29-1 RW P7
  misc_feature (35)..(38): n is a, c, g, or t
gtgactggagttcagacgtgtgctcttccgatctnnnnagtgctgtcatctctcaaaa

SEQ ID NO: 15 - DNA, 21 bp, Artificial Sequence
  Primer AW055 (partialP5)
acactctttccctacacgacg

SEQ ID NO: 16 - DNA, 51 bp, Artificial Sequence
  Primer PE1 (P5) AW042
aatgatacggcgaccaccgagatctacactctttccctacacgacgctctt

SEQ ID NO: 17 - DNA, 51 bp, Artificial Sequence
  Primer PE2 index 1 AW043
caagcagaagacggcatacgagatcgtgatgtgactggagttcagacgtgt

SEQ ID NO: 18 - DNA, 51 bp, Artificial Sequence
  Primer PE2 index 2  AW044
caagcagaagacggcatacgagatacatcggtgactggagttcagacgtgt

SEQ ID NO: 19 - DNA, 51 bp, Artificial Sequence
  Primer PE2 index 3 AW045
caagcagaagacggcatacgagatgcctaagtgactggagttcagacgtgt

SEQ ID NO: 20 - DNA, 51 bp, Artificial Sequence
  Primer PE2 index 4 AW046
caagcagaagacggcatacgagattggtcagtgactggagttcagacgtgt

SEQ ID NO: 21 - DNA, 51 bp, Artificial Sequence
  Primer PE2 index 5 AW047
caagcagaagacggcatacgagatcactgtgtgactggagttcagacgtgt